(19) United States
(12) Patent Application Publication
(10) Pub. No.: US 2006/0069404 A1
(43) Pub. Date: Mar. 30, 2006

(54) Access device having discrete visualization locations

(76) Inventors: Alan E. Shluzas, West Roxbury, MA (US); Gene P. DiPoto, Upton, MA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/241,811

(22) Filed: Sep. 30, 2005

Related U.S. Application Data
(63) Continuation-in-part of application No. 11/094,822, filed on Mar. 30, 2005.
(60) Provisional application No. 60/558,296, filed on Mar. 31, 2004.

(51) Int. Cl. A61M 29/00

(52) U.S. Cl. 606/198

(57) ABSTRACT

A device for providing access to a surgical location within a patient comprises an elongate body having a proximal portion and a distal portion. The elongate body defines a first passage for accessing the surgical location with surgical instruments. The elongate body has a contracted configuration for insertion into the patient and an expanded configuration for providing access to the surgical location. The cross-sectional area of the first passage at a first location of the expanded configuration is greater than the cross-sectional area of the first passage at a second location of the expanded configuration. A second passage is formed integrally with the elongate body. The second passage extends from the proximal portion toward the distal portion and is sized and configured to receive a surgical instrument, such as a viewing element for visualizing the surgical location. The second passage is partially enclosed by a wall of the proximal portion.

### RELATED APPLICATIONS

This application is a continuation-in-part of U.S. patent application Ser. No. 11/094,822 filed Mar. 30, 2005, which claims the benefit of U.S. Provisional Patent Application No. 60/558,296, filed on Mar. 31, 2004, both of which are hereby expressly incorporated by reference herein in their entireties.

### BACKGROUND OF THE INVENTION

1. Field of the Invention

This application is directed to an access assembly for a surgical system that can be actuated from a low-profile configuration for insertion to an enlarged configuration after being inserted. More particularly, this application is directed to a system having an access device that is configured with discrete visualization locations.

2. Description of the Related Art

Spinal surgery presents significant difficulties to the physician attempting to reduce chronic back pain or correct spinal deformities without introducing additional trauma due to the surgical procedure itself. In order to access the vertebrae to perform spinal procedures, the physician is typically required to make large incisions and cut or strip muscle tissue surrounding the spine. In addition, care must be taken not to injure nerve tissue in the area. Consequently, traditional surgical procedures of this type carry high risks of scarring, pain, significant blood loss, and extended recovery times.

Apparatuses for performing minimally invasive techniques have been proposed to reduce the trauma of posterior spinal surgery by reducing the size of the incision and the degree of muscle stripping in order to access the vertebrae. One such apparatus provides a constant diameter cannula that is made narrow in order to provide a small entry profile. As a result, the cannula provides minimal space for the physician to observe the body structures and manipulate surgical instruments in order to perform the required procedures. A narrow cannula is typically insufficient to perform one level spinal fixation procedures, which requires visualization of two vertebrae and introduction of screws, rods, as well as other large spinal fixation devices.

### SUMMARY OF THE INVENTION

Accordingly, there is a need in the art for systems and methods for treating the spine that provide minimally invasive access to the spine such that a variety of procedures, and preferably the entire procedure, can be performed via a single access device. More particularly, there is a need for an access device that is configured with discrete visualization locations. In some embodiments, access devices include visualization passages for receiving viewing elements.

In one embodiment, a device for providing access to a surgical location within a patient comprises an elongate body having a proximal portion and a distal portion. The elongate body defines a first passage for accessing the surgical location with surgical instruments. The elongate body has a contracted configuration for insertion into the patient and an expanded configuration for providing access to the surgical location. The cross-sectional area of the first passage at a first location of the expanded configuration is greater than the cross-sectional area of the first passage at a second location of the expanded configuration. A second passage is separate from the first passage and is formed integrally with the elongate body. The second passage extends from the proximal portion toward the distal portion and is sized and configured to receive a viewing element for visualizing the surgical location.

In another embodiment, a method comprises providing a device comprising an elongate body having a proximal portion and a distal portion. The elongate body defines a first passage and a second passage. The first passage extends through the elongate body through which surgical instruments can be inserted to the surgical location. The second passage is located along a perimeter of the first passage at a first location. The second passage is configured to receive a viewing element. The elongate body is configurable to have an expanded configuration. The elongate body is configured for insertion into the patient.

In another embodiment, a method comprises providing a device comprising an elongate body having a proximal portion and a distal portion. The elongate body defines a first passage and a second passage. The first passage extends through the elongate body through which surgical instruments can be inserted to the surgical location. The second passage is located along a perimeter of the first passage at a first location. The second passage is configured to receive a viewing element. The elongate body is configurable to have an expanded configuration. The elongate body is configured for insertion into the patient. The device is inserted into the patient to the surgical location. The device is expanded to the expanded configuration, such that the cross-sectional area of the first passage at a first location is greater than the cross-sectional area of the first passage at a second location, wherein the first location is distal to the second location. A viewing element is positioned in the second passage.

In another embodiment, a system for accessing and visualizing a surgical location comprises an access device having an elongate body defining a first access passage for accessing the surgical location with surgical instruments and a second access passage for visualizing the surgical location with a viewing element. The second access passage is separate from the first access passage. The elongate body has a contracted configuration for insertion into the patient and an expanded configuration for providing access to the surgical location. A mount fixture is configured to be coupled to the access device. The mount fixture defines a first fixture passage configured to be aligned with said first access passage and a second fixture passage configured to be aligned with said second access passage. A viewing element is configured to be coupled to the mount fixture. The viewing element is configured to be inserted into the second access passage and the second fixture passage.

In another embodiment, a device for providing access to a surgical location within a patient comprises an elongate body having a proximal portion and a distal portion. The elongate body defines a first passage for accessing the surgical location with surgical instruments. The elongate body has a contracted configuration for insertion into the patient and an expanded configuration for providing access to the surgical location. The cross-sectional area of the first passage at a first location of the expanded configuration is greater than the cross-sectional area of the first passage at a second location of the expanded configuration. A second passage is formed integrally with the elongate body. The second passage extends from the proximal portion toward the distal portion and is sized and configured to receive a viewing element for visualizing the surgical location. The second passage is partially enclosed by a wall of the proximal portion.

In another embodiment, a system for accessing and visualizing a surgical location comprises a device having an elongate body defining a first passage for accessing the surgical location with surgical instruments and a second passage for visualizing the surgical location with a viewing element. The elongate body has a proximal portion and a distal portion. The elongate body has a contracted configuration for insertion into the patient and an expanded configuration for providing access to the surgical location. The second passage is partially enclosed by a wall of the proximal portion. A viewing element is configured to be inserted into the second passage.

In another application, a method comprises providing a device comprising an elongate body having a proximal portion and a distal portion. The elongate body defines a first passage and a second passage. The first passage extends through the elongate body through which surgical instruments can be inserted to a surgical location of a patient. The second passage is partially enclosed by a wall of said proximal portion. The second passage is configured to receive a viewing element. The elongate body is configurable to have an expanded configuration. The elongate body is configured for insertion into the patient.

In another embodiment, a device for providing access to a surgical location within a patient comprises an elongate body having a proximal portion and a distal portion. The elongate body defines a first passage for accessing the surgical location with surgical instruments. The elongate body has a contracted configuration for insertion into the patient and an expanded configuration for providing access to the surgical location. The cross-sectional area of said first passage at a first location of the expanded configuration is greater than the cross-sectional area of said first passage at a second location of the expanded configuration. A second passage is formed integrally with the elongate body. The second passage extends at least partially from the proximal portion toward the distal portion and is sized and configured to receive a surgical instrument. The second passage is partially enclosed by a wall at least in the proximal portion.

In another embodiment, a system for accessing and visualizing a surgical location comprises a device having an elongate body defining a first passage for accessing the surgical location with surgical instruments and a second passage for visualizing the surgical location with a viewing element. The elongate body has a proximal portion and a distal portion. The elongate body has a contracted configuration for insertion into the patient and an expanded configuration for providing access to the surgical location. The second passage is partially enclosed by a wall at least in the proximal portion. A surgical instrument is configured to be inserted into the second passage.

In another application, a method of accessing a surgical location comprises providing a device comprising an elongate body having a proximal portion and a distal portion. The elongate body defines a first passage and a second passage. The first passage extends through the elongate body through which surgical instruments can be inserted to a surgical location of a patient. The second passage is partially enclosed by a wall at least in said proximal portion. The second passage is configured to receive a surgical instrument. The elongate body is configurable to have an expanded configuration. The elongate body is configured in the expanded configuration for insertion into the patient.

### BRIEF DESCRIPTION OF THE DRAWINGS

Further objects, features and advantages of the invention will become apparent from the following detailed description taken in conjunction with the accompanying figures showing illustrative embodiments of the invention, in which:

FIG. 1 is a perspective view of one embodiment of a surgical system and one application for treating the spine of a patient.

FIG. 2 is a perspective view of one embodiment of an access device in a reduced profile configuration.

FIG. 3 is a perspective view of the access device of FIG. 2 in a first enlarged configuration.

FIG. 4 is a perspective view of the access device of FIG. 2 in a second enlarged configuration.

FIG. 5 is a view of one embodiment of a skirt portion of an access device.

FIG. 6 is a view of another embodiment of a skirt portion of an access device.

FIG. 7 is a perspective view of another embodiment of an access device.

FIG. 8 is a side view of the access device of FIG. 7.

FIG. 9 is a front view of the access device of FIG. 7.

FIG. 10 is a bottom view of the access device of FIG. 7.

FIG. 11 is a perspective view of the access device of FIG. 7 in a first configuration.

FIG. 12 is an exploded perspective view of the access device of FIG. 7 in a second configuration.

FIG. 13 is a sectional view illustrating one stage of one application for treating the spine of a patient.

FIG. 14 is a side view of one embodiment of an expander apparatus in a reduced profile configuration.

FIG. 15 is a side view of the expander apparatus of FIG. 14 in an expanded configuration.

FIG. 16 is a sectional view of the expander apparatus of FIGS. 14-15 inserted into the access device of FIG. 2, which has been inserted into a patient.

FIG. 17 is a sectional view of the expander apparatus of FIGS. 14-15 inserted into the access device of FIG. 2 and expanded to the expanded configuration to retract tissue.

FIG. 18 is an exploded perspective view of one embodiment of an endoscope mount platform.

FIG. 19 is a top view of the endoscope mount platform of FIG. 18 coupled with one embodiment of an indexing arm and one embodiment of an endoscope.

FIG. 20 is a side view of the endoscope mount platform of FIG. 18 illustrated with one embodiment of an indexing arm and one embodiment of an endoscope.

FIG. 21 is a perspective view of one embodiment of an indexing collar of the endoscope mount platform FIG. 18.

FIG. 22 is a perspective view of one embodiment of an endoscope.

FIG. 23A is a top perspective view of one embodiment of an access system.

FIG. 23B is a side perspective view of the access system of FIG. 23A.

FIG. 23C is a top view of the access system of FIG. 23A.

FIG. 24A is a perspective view of one embodiment of a lighting element.

FIG. 24B is a perspective view of another embodiment of a lighting element.

FIG. 24C is a perspective view of another embodiment of a lighting element.

FIG. 25 is a partial sectional view of one stage of one application of a method for treating the spine of a patient.

FIG. 26 is a perspective view of one embodiment of a fastener.

FIG. 27 is an exploded perspective view of the fastener of FIG. 26.

FIG. 27A is an enlarged side view of one embodiment of a biasing member illustrated in FIG. 27 taken from the perspective of the arrow 27A.

FIG. 28 is a perspective view of one embodiment of a surgical instrument.

FIG. 29 is an enlarged sectional view of the fastener of FIGS. 26-27 coupled with the surgical instrument of FIG. 28, illustrating one stage of one application for treating the spine of a patient.

FIG. 30 is side view of one embodiment of another surgical instrument.

FIG. 31 is a partial sectional view of one stage of one application for treating the spine of a patient.

FIG. 32 is a side view of one embodiment of another surgical instrument.

FIG. 33 is a perspective view similar to FIG. 31 illustrating the apparatuses of FIGS. 26 and 32, in one stage of one application for treating the spine of a patient.

FIG. 34 is an enlarged sectional view of the apparatus of FIGS. 26 and 32, illustrating one stage of one application for treating the spine of a patient.

FIG. 35 is an enlarged sectional similar to FIG. 34, illustrating one stage of one application for treating the spine of a patient.

FIG. 36 is an enlarged view in partial section illustrating one stage of one application for treating the spine of a patient.

FIG. 37 is a partial view of illustrating one stage of one application for treating the spine of a patient.

FIG. 38 is a perspective view of a spinal implant or fusion device constructed according to another embodiment showing a first side surface of the spinal implant.

FIG. 39 is a perspective view of the spinal implant of FIG. 38 showing a second side surface of the spinal implant.

FIG. 40 is a plan view of the spinal implant of FIG. 38 showing an upper surface of the spinal implant.

FIG. 41 is a side view of the spinal implant of FIG. 38 showing the first side surface.

FIG. 42 is a cross-sectional view of the spinal implant taken along the line 42-42 in FIG. 41.

FIG. 43 is a perspective view of another embodiment of a spinal implant constructed according to another embodiment showing a first side surface of the spinal implant.

FIG. 44 is a perspective view of the spinal implant of FIG. 43 showing a second side surface of the spinal implant.

FIG. 45 is a plan view of the spinal implant of FIG. 43 showing an upper surface of the spinal implant.

FIG. 46 is a side view of the spinal implant of FIG. 43 showing the first side surface.

FIG. 47 is a cross-sectional view of the spinal implant taken along the line 47-47 in FIG. 46.

FIG. 48 is a view showing a pair of the spinal implants of FIG. 38 in first relative positions between adjacent vertebrae.

FIG. 49 is a view showing a pair of the spinal implants of FIG. 38 in second relative positions between adjacent vertebrae.

FIG. 50 is a view showing the spinal implant of FIG. 43 between adjacent vertebrae.

FIG. 51 is a view showing a spinal implant being inserted between the adjacent vertebrae according to one application.

FIG. 52 is a side view of an apparatus according to another embodiment.

FIG. 53 is a front view of the apparatus of FIG. 52.

FIG. 54 is a top view of the apparatus of FIG. 52.

FIG. 55 is a back view of the apparatus of FIG. 52.

FIG. 56 is a bottom view of the apparatus of FIG. 52.

FIG. 57 is a sectional view of the apparatus of FIG. 52, used in conjunction with additional structure in a patient.

FIG. 58 is a longitudinal sectional view of the apparatus of FIG. 57 taken from line 58-58 of FIG. 57.

FIG. 59 is a transverse sectional view of the apparatus of FIG. 58 taken from line 59-59 of FIG. 58.

FIG. 60 is a sectional view, similar to FIG. 57, illustrating an alternative position of the apparatus of FIG. 52.

FIG. 61 is a sectional view, similar to FIG. 57, illustrating another alternative position of the apparatus of FIG. 52.

FIG. 62 is a transverse sectional view of the apparatus of FIG. 61, taken along lines 62-62 of FIG. 61.

FIG. 63 is a side view, similar to FIG. 52, of another apparatus.

FIG. 64 is a front view, similar to FIG. 55, of the embodiment of FIG. 63.

FIG. 65 is a sectional view, similar to FIG. 57, of the apparatus of FIG. 63, used in conjunction with additional structure in a patient.

FIG. 66 is a transverse sectional view of the apparatus of FIG. 63, taken along lines 66-66 of FIG. 65.

FIG. 67 is a perspective view of one embodiment of an access device.

FIG. 68 is a perspective sectional view of the access device of FIG. 67, taken along line 68-68 of FIG. 67.

FIG. 69 is a front view of the access device of FIG. 67.

FIG. 70 is a top view of the access device of FIG. 67.

FIG. 71 is a side view of the access device of FIG. 67.

FIG. 72 is a perspective view of one embodiment of an access assembly, including the access device of FIG. 67.

FIG. 73 is a front view of the access assembly of FIG. 72.

FIG. 74 is a top view of the access assembly of FIG. 72.

FIG. 75 is a side view of the access assembly of FIG. 72.

FIG. 76 is an exploded perspective view of the access assembly of FIG. 72.

FIG. 76A is a cross-sectional view of a portion of the access assembly of FIG. 76, taken along line 76A-76A of FIG. 76.

FIG. 77 is a perspective view of another embodiment of an access assembly capable of being positioned in at least two viewing positions.

FIG. 78 is an exploded perspective view of the access assembly of FIG. 77.

FIG. 79 is a top view of the access assembly of FIG. 77 in a first viewing position, with a second viewing position shown in dashed line.

FIG. 80 is another embodiment of an access device in an expanded configuration.

FIG. 81 is a side view of the access device of FIG. 80 in a low profile configuration, with an expanded configuration shown in dashed line.

FIG. 82 is a side view of another embodiment of an access assembly, including the access device of FIG. 80.

FIG. 83 is a side view of another embodiment of an access assembly, including the access device of FIG. 80.

FIG. 84 is a top view of the access assembly of FIG. 83.

FIG. 85 is a side view of the access device of FIG. 80, with one variation shown in dashed line.

FIG. 86 is another embodiment of an access device.

FIG. 87 is a cross-sectional view of another embodiment of an access device with exterior visualization channels.

FIG. 88 is another embodiment of an access assembly with a viewing element support mount coupled to a central portion of an access device.

FIG. 89 is yet another embodiment of an access assembly with a viewing element coupled to a central portion of an access device.

FIG. 90 is an elevational view of another embodiment of an access assembly including an access device and support elements.

FIG. 91 is an elevational view of another embodiment of an access assembly including an access device and support elements.

FIG. 92 is a back perspective view of another embodiment of an access device.

FIG. 93 is a front perspective view of the access device of FIG. 92.

FIG. 94 is a front view of the access device of FIG. 92.

FIG. 95 is a top view of the access device of FIG. 92.

FIG. 96 is a side view of the access device of FIG. 92.

FIG. 97 is a perspective view of the access device of FIG. 92 with an endoscope inserted into a viewing element passage.

FIG. 98 is another perspective view of the access device of FIG. 92 with an endoscope inserted into a viewing element passage.

Throughout the figures, the same reference numerals and characters, unless otherwise stated, are used to denote like features, elements, components or portions of the illustrated embodiments. Moreover, while the subject invention will now be described in detail with reference to the figures, it is done so in connection with the illustrative embodiments. It is intended that changes and modifications can be made to the described embodiments without departing from the true scope and spirit of the subject invention.

### DETAILED DESCRIPTION OF THE PREFERRED EMBODIMENT

As should be understood in view of the following detailed description, this application is primarily directed to apparatuses and methods providing access to and for treating the spine of a patient. The apparatuses described below provide access to surgical locations at or near the spine and provide a variety of tools useful treating the spine. In particular, various embodiments described hereinbelow include access devices that are particularly well adapted to be coupled with one or more viewing elements. In some embodiments, access devices are provided that are configured to receive one or more viewing elements at discrete locations about a passage defined by the access device. The apparatuses described herein enable a surgeon to perform a wide variety of methods of treatment as described herein.

### I. Systems for Performing Procedures at a Surgical Location

Various embodiments of apparatuses and procedures described herein will be discussed in terms of minimally invasive procedures and apparatuses, e.g., of endoscopic apparatuses and procedures. However, various embodiments may find use in conventional, open, and mini-open procedures. As used herein, the term “proximal,” as is traditional, refers to the end portion of an apparatus that is closest to the operator, while the term “distal” refers to the end portion that is farthest from the operator.

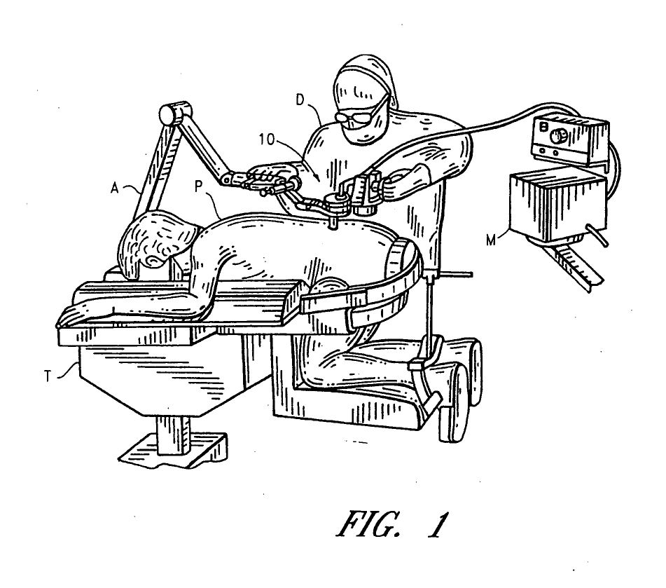

FIG. 1 shows one embodiment of a surgical system 10 that can be used to perform a variety of methods or procedures. In one embodiment, as discussed more fully below, the patient P is placed in the prone position on operating table T, taking care that the abdomen is not compressed and physiological lordosis is preserved. The physician D is able to access the surgical site and perform the surgical procedure with the components of the system 10, which will be described in greater detail herein. The system 10 may be supported, in part, by a mechanical support arm A, such as the type generally disclosed in U.S. Pat. No. 4,863,133, which is hereby incorporated by reference herein in its entirety. One mechanical arm of this type is manufactured by Leonard Medical, Inc., 1464 Holcomb Road, Huntington Valley, Pa., 19006. The mechanical support arm A is sometimes referred to as a “flex arm.” As discussed in greater detail below, the mechanical support arm A is coupled with at least one of an access device and a viewing element.

The term “access device” is used in its ordinary sense to mean a device that can provide access and is a broad term and it includes structures having an elongated dimension and defining a passage, e.g., a cannula or a conduit. The access device is configured to be inserted through the skin of the patient to provide access during a surgical procedure to a surgical location within a patient, e.g., a spinal location. The term “surgical location” is used in its ordinary sense (i.e. a location where a surgical procedure is performed) and is a broad term and it includes locations subject to or affected by a surgery. The term “spinal location” is used in its ordinary sense (i.e. a location at or near a spine) and is a broad term and it includes locations adjacent to or associated with a spine that may be sites for surgical spinal procedures. The access device also can retract tissue to provide greater access to the surgical location. The term “retractor” is used in its ordinary sense to mean a device that can displace tissue and is a broad term and it includes structures having an elongated dimension and defining a passage, e.g., a cannula or a conduit, to retract tissue.

Visualization of the surgical site may be achieved in any suitable manner, e.g., by direct visualization, or by use of a viewing element, such as an endoscope, a camera, loupes, a microscope, or any other suitable viewing element, or a combination of the foregoing. The term “viewing element” is used in its ordinary sense to mean a device useful for viewing and is a broad term and it also includes elements that enhance viewing, such as, for example, a light source or lighting element. In one embodiment, the viewing element provides a video signal representing images, such as images of the surgical site, to a monitor M. The viewing element may be an endoscope and camera that captures images to be displayed on the monitor M whereby the physician D is able to view the surgical site as the procedure is being performed. The endoscope and camera will be described in greater detail herein.

The systems are described herein in connection with minimally invasive postero-lateral spinal surgery. One such procedure is a two level postero-lateral fixation and fusion of the spine involving the L4, L5, and S1 vertebrae. In the drawings, the vertebrae will generally be denoted by reference letter V. The usefulness of the apparatuses and procedures is neither restricted to the postero-lateral approach nor to the L4, L5, and S1 vertebrae. The apparatuses and procedures may be used in other anatomical approaches and with other vertebra(e) within the cervical, thoracic, and lumbar regions of the spine. The procedures may be directed toward surgery involving one or more vertebral levels. Some embodiments are useful for anterior and/or lateral procedures. A retroperitoneal approach can also be used with some embodiments. In one retroperitoneal approach, an initial transverse incision is made just left of the midline, just above the pubis, about 3 centimeters in length. The incision can be carried down through the subcutaneous tissues to the anterior rectus sheath, which is incised transversely and the rectus is retracted medially. At this level, the posterior sheath, where present, can be incised. With blunt finger dissection, the retroperitoneal space can be entered. The space can be enlarged with blunt dissection or with a retroperitoneal balloon dissector. The peritoneal sack can be retracted, e.g., by one of the access devices described herein.

It is believed that embodiments of the invention are also particularly useful where any body structures must be accessed beneath the skin and muscle tissue of the patient, and/or where it is desirable to provide sufficient space and visibility in order to manipulate surgical instruments and treat the underlying body structures. For example, certain features or instrumentation described herein are particularly useful for minimally invasive procedures, e.g., arthroscopic procedures. As discussed more fully below, one embodiment of an apparatus described herein provides an access device that is expandable, e.g., including an expandable distal portion. In addition to providing greater access to a surgical site than would be provided with a device having a constant cross-section from proximal to distal, the expandable distal portion prevents or substantially prevents the access device, or instruments extended therethrough to the surgical site, from dislodging or popping out of the operative site.

A. Systems and Devices for Establishing Access

In one embodiment, the system 10 includes an access device that provides an internal passage for surgical instruments to be inserted through the skin and muscle tissue of the patient P to the surgical site. The access device preferably has a wall portion defining a reduced profile, or low-profile, configuration for initial percutaneous insertion into the patient. This wall portion may have any suitable arrangement. In one embodiment, discussed in more detail below, the wall portion has a generally tubular configuration that may be passed over a dilator that has been inserted into the patient to atraumatically enlarge an opening sufficiently large to receive the access device therein.

The wall portion of the access device preferably can be subsequently expanded to an enlarged configuration, by moving against the surrounding muscle tissue to at least partially define an enlarged surgical space in which the surgical procedures will be performed. In a sense, it acts as its own dilator. The access device may also be thought of as a retractor, and may be referred to herein as such. Both the distal and proximal portion may be expanded, as discussed further below. However, the distal portion preferably expands to a greater extent than the proximal portion, because the surgical procedures are to be performed at the surgical site, which is adjacent the distal portion when the access device is inserted into the patient.

While in the reduced profile configuration, the access device preferably defines a first unexpanded configuration. Thereafter, the access device can enlarge the surgical space defined thereby by engaging the tissue surrounding the access device and displacing the tissue outwardly as the access device expands. The access device preferably is sufficiently rigid to displace such tissue during the expansion thereof. The access device may be resiliently biased to expand from the reduced profile configuration to the enlarged configuration. In addition, the access device may also be manually expanded by an expander device with or without one or more surgical instruments inserted therein, as will be described below. The surgical site preferably is at least partially defined by the expanded access device itself. During expansion, the access device can move from a first overlapping configuration to a second overlapping configuration in some embodiments.

In some embodiments, the proximal and distal portions are separate components that may be coupled together in a suitable fashion. For example, the distal end portion of the access device may be configured for relative movement with respect to the proximal end portion in order to allow the physician to position the distal end portion at a desired location. This relative movement also provides the advantage that the proximal portion of the access device nearest the physician D may remain substantially stable during such distal movement. In one embodiment, the distal portion is a separate component that is pivotally or movably coupled to the proximal portion. In another embodiment, the distal portion is flexible or resilient in order to permit such relative movement.

1. Access Devices

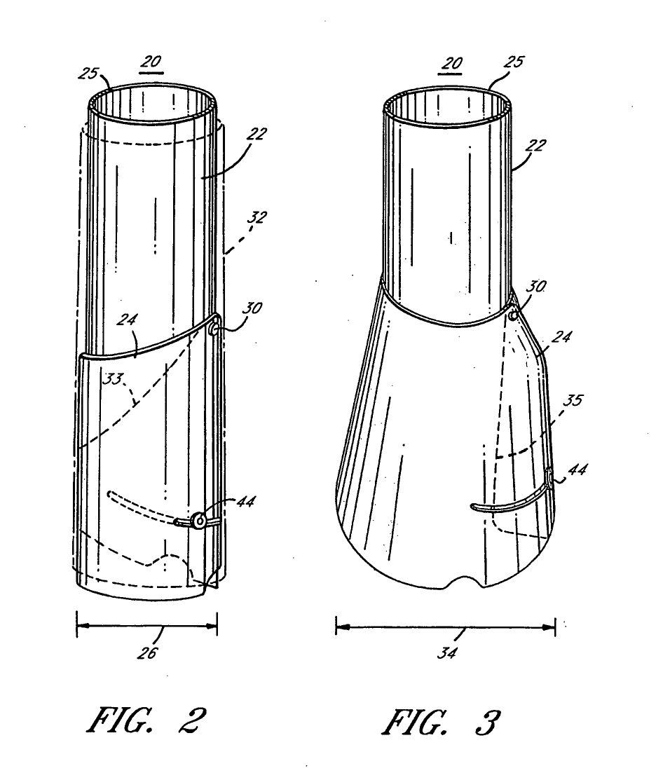

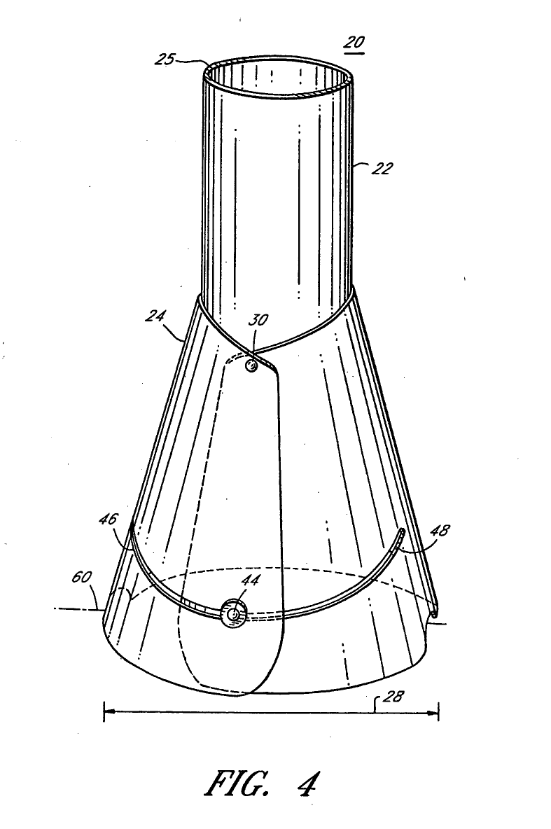

One embodiment of an access device is illustrated in FIGS. 2-6 and designated by reference number 20. In one embodiment, the access device 20 includes a proximal wall portion 22 that has a tubular configuration, and a distal wall portion that has an expandable skirt portion 24. The skirt portion 24 preferably is enlargeable from a reduced profile configuration having an initial dimension 26 (illustrated in FIG. 2) and corresponding cross-sectional area, to an enlarged configuration having a second dimension 28 (illustrated in FIG. 4) and corresponding cross-sectional area. In one embodiment, the skirt portion 24 is coupled to the proximal wall portion 22 with a rivet 30, pin, or similar connecting device to permit movement of the skirt portion 24 relative to the proximal wall portion 22.

In the illustrated embodiment, the skirt portion 24 is manufactured from a resilient material, such as stainless steel. The skirt portion 24 preferably is manufactured so that it normally assumes an expanded configuration as illustrated in FIG. 4. With reference to FIG. 3, the skirt portion 24 may assume an intermediate dimension 34 and corresponding cross-sectional area, which is greater than the initial dimension 26 of the reduced profile configuration of FIG. 2, and smaller than the dimension 28 of the enlarged configuration of FIG. 4. The skirt portion 24 may assume the intermediate configuration of FIG. 3 when deployed in the patient in response to the force of the tissue acting on the skirt portion 24. The intermediate dimension 34 can depend upon several factors, such as the rigidity of the skirt portion 24, the surrounding tissue, and whether such surrounding tissue has relaxed or tightened during the course of the procedure. An outer sleeve 32 (illustrated in dashed line in FIG. 2) may be provided. Preferably, the outer sleeve surrounds the access device 20 and maintains the skirt portion 24 in the reduced profile configuration prior to insertion into the patient. The outer sleeve 32 may be made of plastic. Where provided, the outer sleeve 32 preferably is configured to be easily deployed. For example, a release device may be provided that releases or removes the outer sleeve 32 upon being operated by the user. In one embodiment, a braided polyester suture is embedded within the sleeve 32, aligned substantially along the longitudinal axis thereof. In use, when the suture is withdrawn, the outer sleeve 32 is torn, allowing the access device 20 to resiliently expand from the reduced profile configuration of FIG. 2 to the expanded configurations of FIGS. 3-4. While in the reduced profile configuration of FIG. 2, the skirt portion 24 defines a first overlapping configuration 33, as illustrated by the dashed line. As the skirt portion 24 resiliently expands, the skirt portion 24 assumes the expanded configuration, as illustrated in FIGS. 3-4.

The skirt portion 24 preferably is sufficiently rigid that it is capable of displacing the tissue surrounding the skirt portion 24 as it expands. Depending upon the resistance exerted by surrounding tissue, the skirt portion 24 preferably is sufficiently rigid to provide some resistance against the tissue to remain in the configurations of FIGS. 3-4. Moreover, the expanded configuration of the skirt portion 24 is at least partially supported by the body tissue of the patient. The rigidity of the skirt portion 24 and the greater expansion at the distal portion preferably creates a stable configuration that is at least temporarily stationary in the patient. This arrangement preferably frees the physician from the need to actively support the access device 20, e.g., prior to adding an endoscope mount platform 300 and a support arm 400 (see FIGS. 21-22).

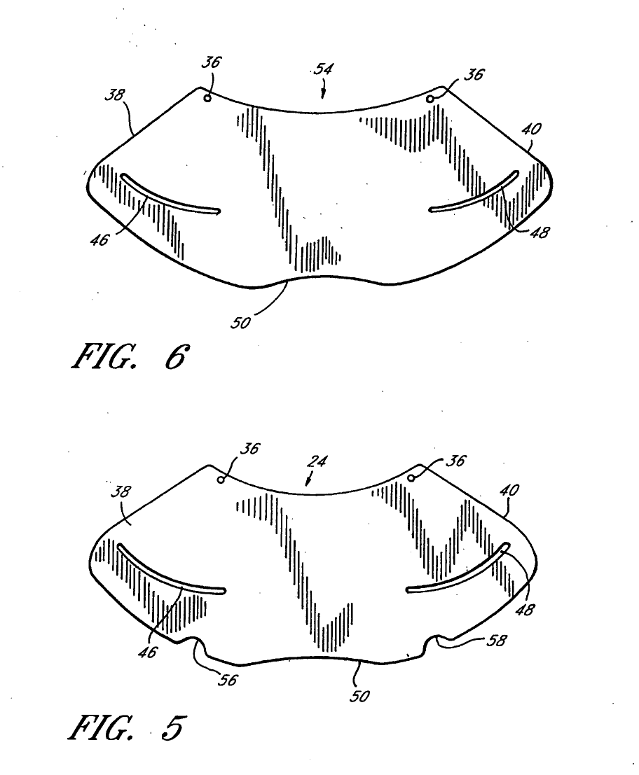

One embodiment of the skirt portion 24 of the access device 20 is illustrated in an initial flattened configuration in FIG. 5. The skirt portion 24 may be manufactured from a sheet of stainless steel having a thickness of about 0.007 inches. In various embodiments, the dimension 28 of the skirt portion 24 is about equal to or greater than 50 mm, is about equal to or greater than 60 mm, is about equal to or greater than 70 mm, is about equal to or greater than 80 mm, or is any other suitable size, when the skirt portion 24 is in the enlarged configuration. In one embodiment, the dimension 28 is about 63 mm, when the skirt portion 24 is in the enlarged configuration. The unrestricted shape of the skirt portion 24 is a circular shape in one embodiment and is an oblong shape in another embodiment. In another embodiment, the skirt portion 24 has an oval shape, wherein the dimension 28 defines a longer dimension of the skirt portion 24 and would be about 85 mm. In another embodiment, the skirt portion 24 has an oval shape and the dimension 28 defines a longer dimension of the skirt portion 24 of about 63 mm. An increased thickness, e.g., about 0.010 inches, may be used in connection with skirt portions having a larger diameter, such as about 65 mm. Other materials, such as nitinol or plastics having similar properties, may also be useful.

As discussed above, the skirt portion 24 preferably is coupled to the proximal wall portion 22 with a pivotal connection, such as rivet 30. A pair of rivet holes 36 can be provided in the skirt portion 24 to receive the rivet 30. The skirt portion 24 also has two free ends 38 and 40 in one embodiment that are secured by a slidable connection, such as a second rivet 44 (not shown in FIG. 5, illustrated in FIGS. 2-4). A pair of complementary slots 46 and 48 preferably are defined in the skirt portion 24 adjacent the free ends 38 and 40. The rivet 44 is permitted to move freely within the slots 46 and 48. This slot and rivet configuration allows the skirt portion 24 to move between the reduced profile configuration of FIG. 2 and the enlarged or expanded configurations of FIGS. 3-4. The use of a pair of slots 46 and 48 reduces the risk of the “button-holing” of the rivet 44, e.g., a situation in which the opening of the slot becomes distorted and enlarged such that the rivet may slide out of the slot, and cause failure of the device. The likelihood of such occurrence is reduced in skirt portion 24 because each of the slots 46 and 48 in the double slot configuration has a relatively shorter length than a single slot configuration. Being shorter, the slots 46, 48 are less likely to be distorted to the extent that a rivet may slide out of position. In addition, the configuration of rivet 44 and slots 46 and 48 permits a smoother operation of enlarging and reducing the skirt portion 24, and allows the skirt portion 24 to expand to span three or more vertebrae, e.g., L4, L5, and S1. This arrangement enables multi-level procedures, such as multilevel fixation procedures alone or in combination with a variety of other procedures, as discussed below. Other embodiments include a single slot rather than the slots 46, 48, or more than two slots.

An additional feature of the skirt portion 24 is the provision of a shallow concave profile 50 defined along the distal edge of the skirt portion 24, which allows for improved placement of the skirt portion 24 with respect to the body structures and the surgical instruments defined herein. In one embodiment, a pair of small scalloped or notched portions 56 and 58, are provided, as illustrated in FIG. 5. When the skirt portion 24 is assembled, the notched portions 56 and 58 are generally across from each other. When the skirt portion 24 is applied to a patient, the notched portions 56, 58 are oriented in the ceph-caudal direction (indicated by a dashed line 60 in FIG. 4). In this arrangement, instruments and implants, such as an elongated member 650 used in a fixation procedure (described in detail below), may extend beyond the area enclosed by the skirt portion 24 without moving or raising the skirt portion 24, e.g., by allowing the elongated member 650 (or other implant or instrument) to pass under the skirt portion 24. The notched portions 56, 58 also enable the elongated member 650 (or other implant or instrument) to extend beyond the portion of the surgical space defined within the outline of the distal end of the skirt portion 24. The notched portions 56, 58 are optional, as illustrated in connection with another embodiment of an access device 54, illustrated in FIG. 6, and may be eliminated if, for example, the physician deems the notches to be unnecessary for the procedures to be performed. For example, in some fixation procedures such extended access is not needed, as discussed more fully below. As illustrated in FIG. 4, the skirt portion 24 may be expanded to a substantially conical configuration having a substantially circular or elliptical profile.

Furthermore, it is contemplated that the skirt portion 24 of the access device 20 can include a stop that retains the skirt portion in an expanded configuration, as shown in U.S. patent application Ser. No. 10/361,887, filed Feb. 10, 2003, now U.S. Application Patent Publication No. U.S. 2003/153927 A1, which is hereby incorporated by reference in its entirety herein.

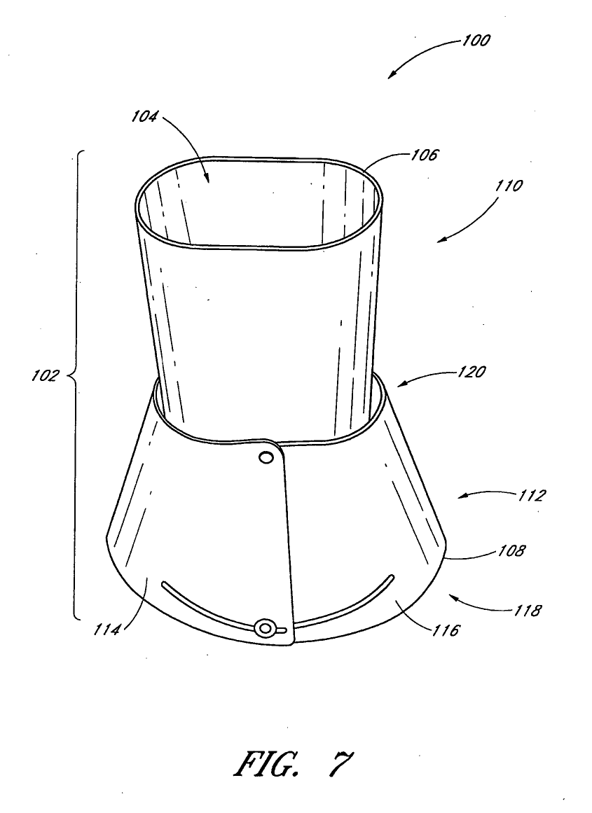

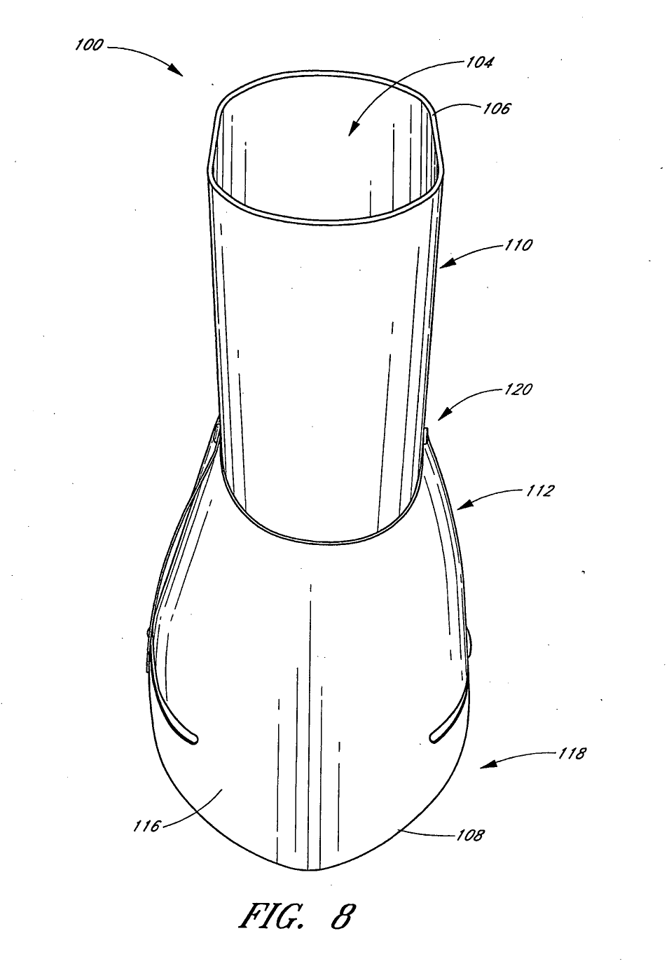

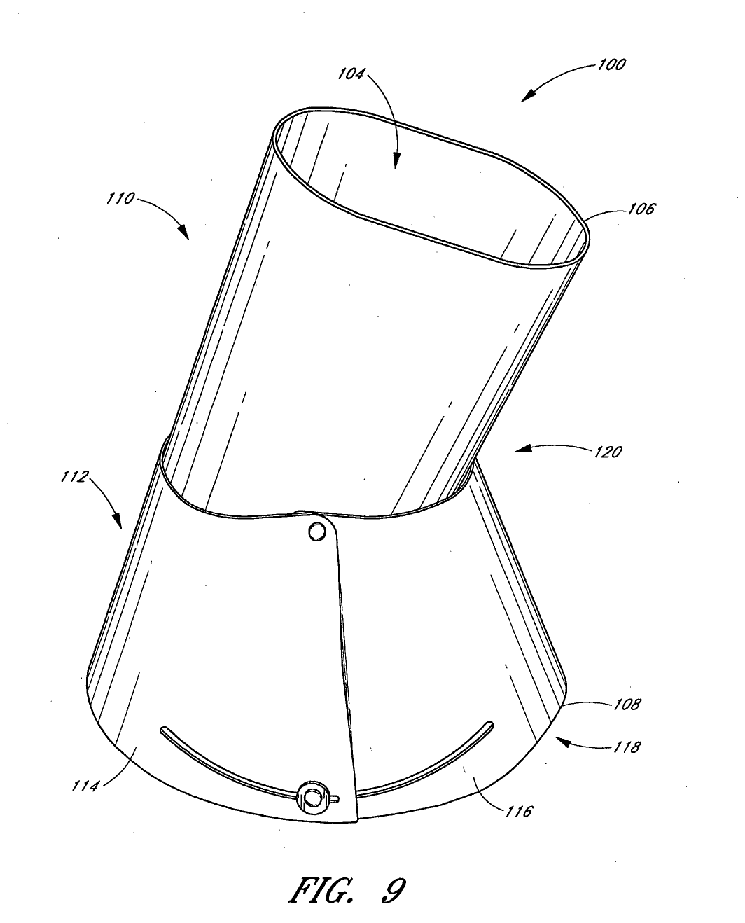

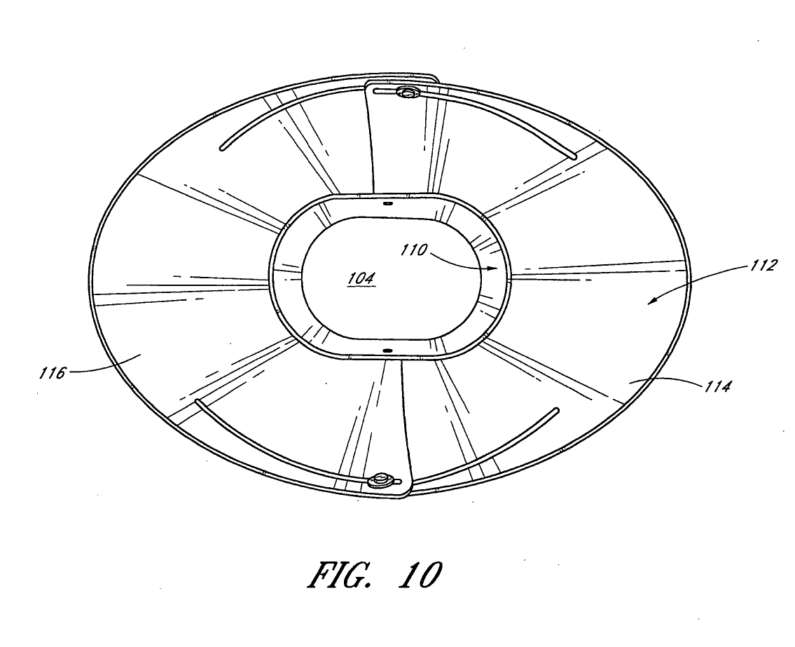

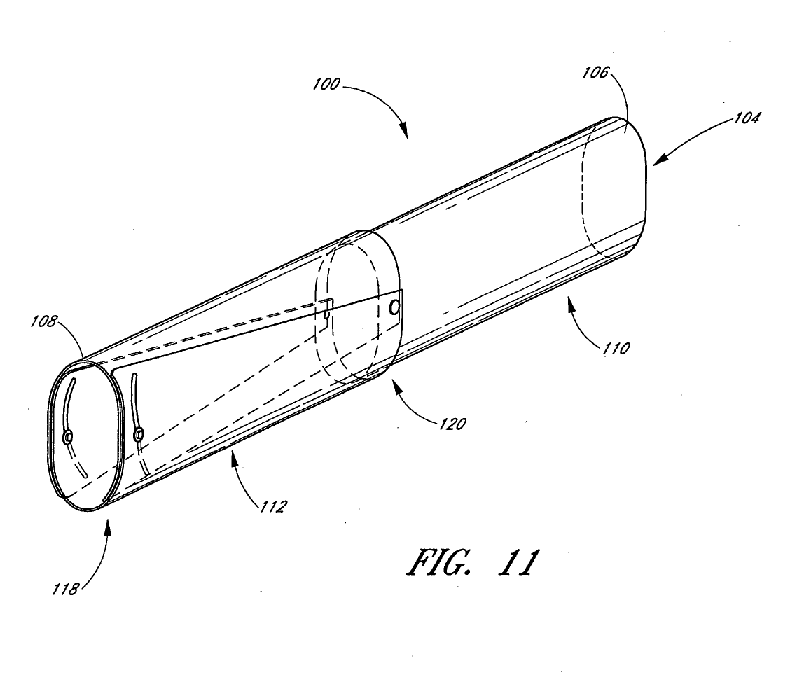

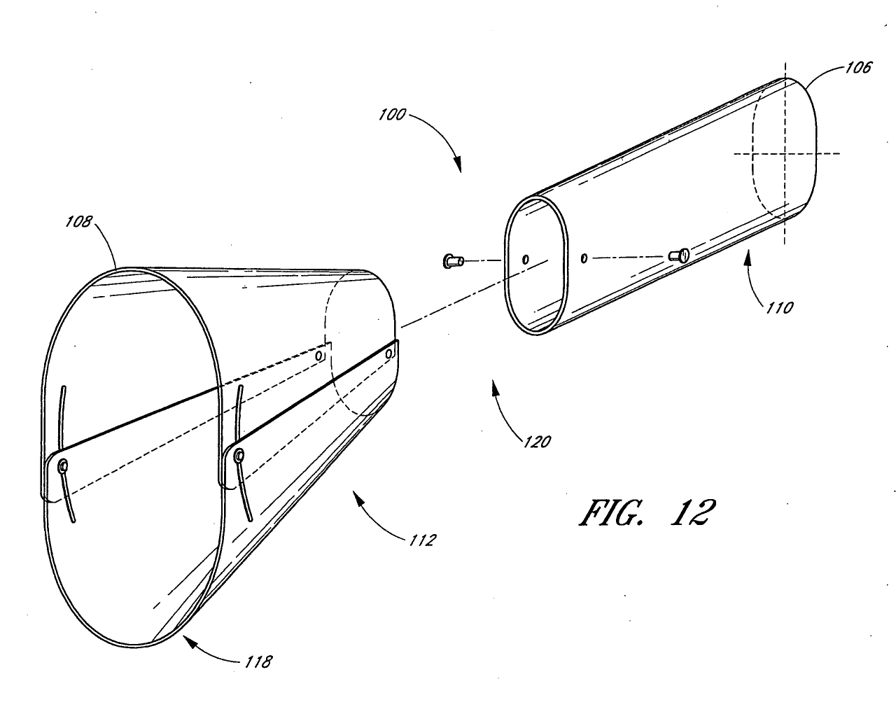

With reference to FIGS. 7-12, another embodiment of an access device 100 comprises an elongate body 102 defining a passage 104 and having a proximal end 106 and a distal end 108. The elongate body 102 has a proximal portion 110 and a distal portion 112. The proximal portion 110 has an oblong or generally oval shaped cross section in one embodiment. The term “oblong” is used in its ordinary sense (i.e., having an elongated form) and is a broad term and it includes a structure having a dimension, especially one of two perpendicular dimensions, such as, for example, width or length, that is greater than another and includes shapes such as rectangles, ovals, ellipses, triangles, diamonds, trapezoids, parabolas, and other elongated shapes having straight or curved sides. The term “oval” is used in its ordinary sense (i.e., egg like or elliptical) and is a broad term and includes oblong shapes having curved portions.

The proximal portion 110 comprises an oblong, generally oval shaped cross section over the elongated portion. It will be apparent to those of skill in the art that the cross section can be of any suitable oblong shape. The proximal portion 110 can be any desired size. The proximal portion 110 can have a cross-sectional area that varies from one end of the proximal portion to another end. For example, the cross-sectional area of the proximal portion can increase or decrease along the length of the proximal portion 110. Preferably, the proximal portion 110 is sized to provide sufficient space for inserting multiple surgical instruments through the elongate body 102 to the surgical location. The distal portion 112 preferably is expandable and comprises first and second overlapping skirt members 114, 116. The degree of expansion of the distal portion 112 is determined by an amount of overlap between the first skirt member 114 and the second skirt member 116 in one embodiment.

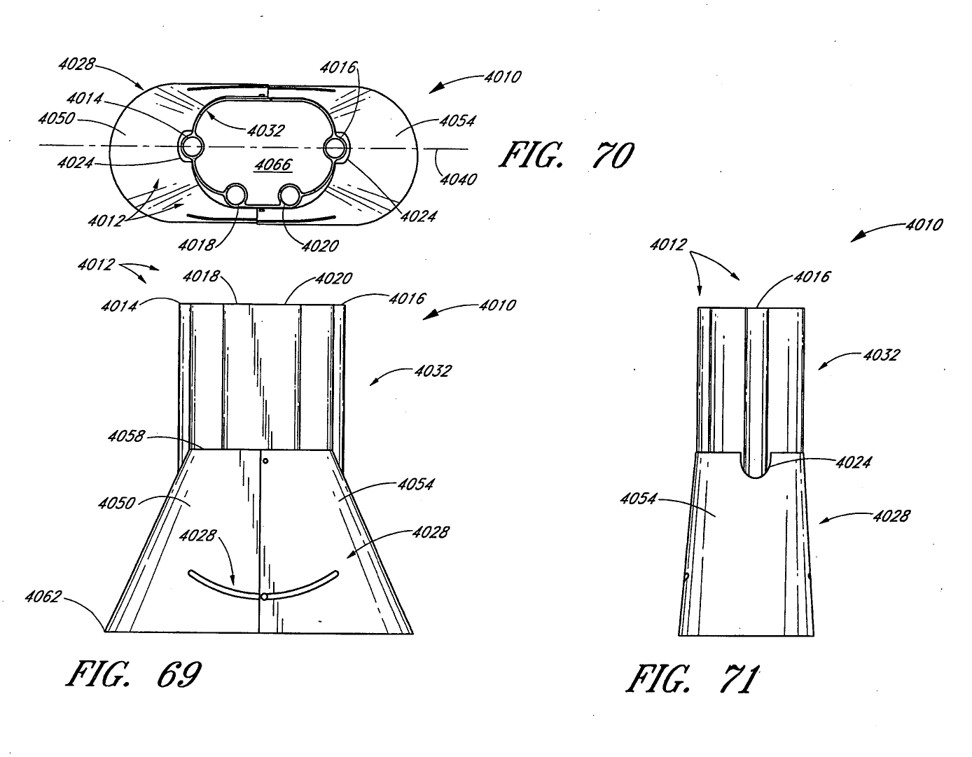

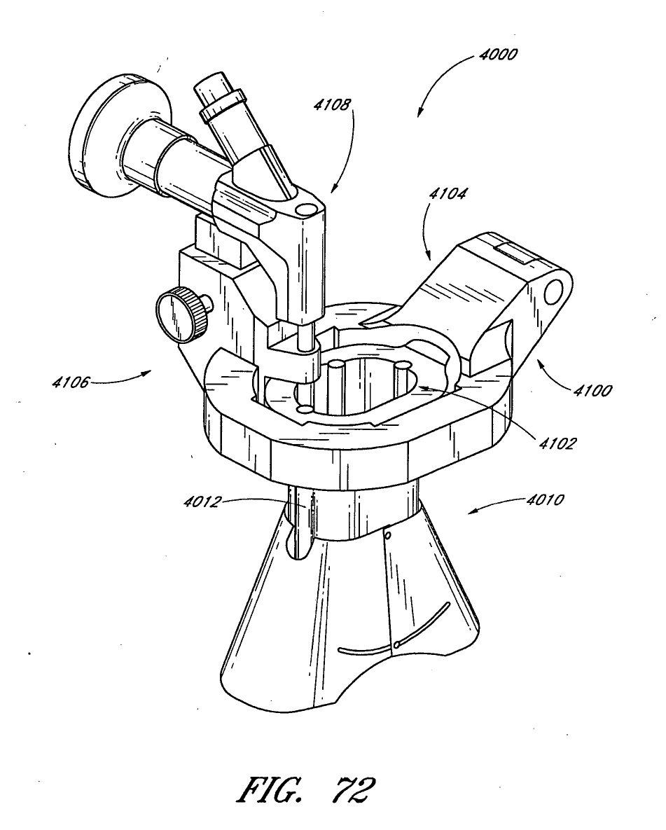

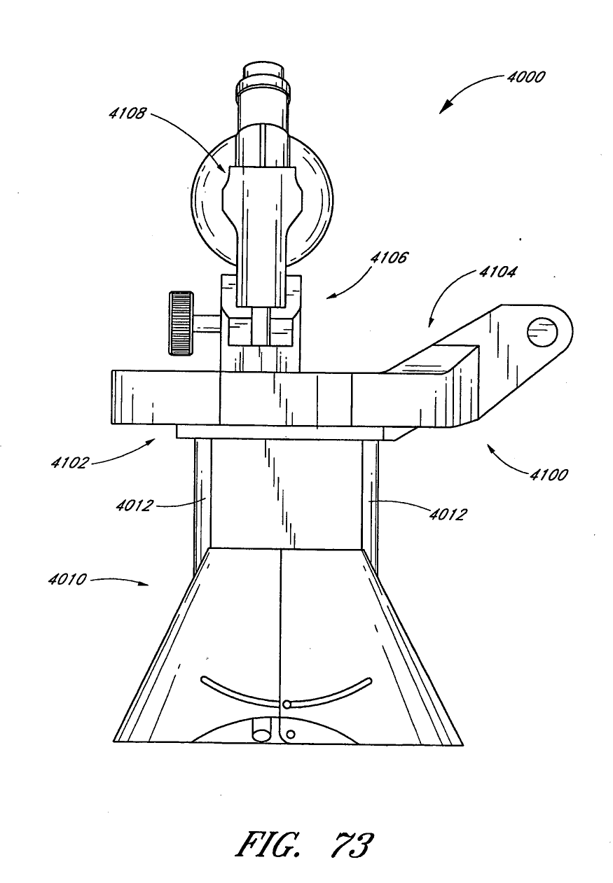

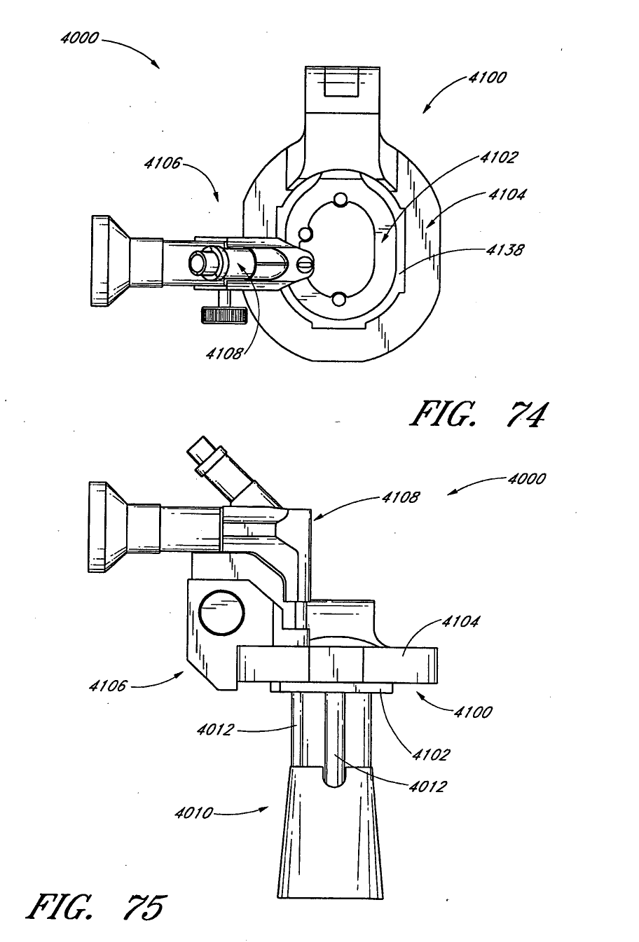

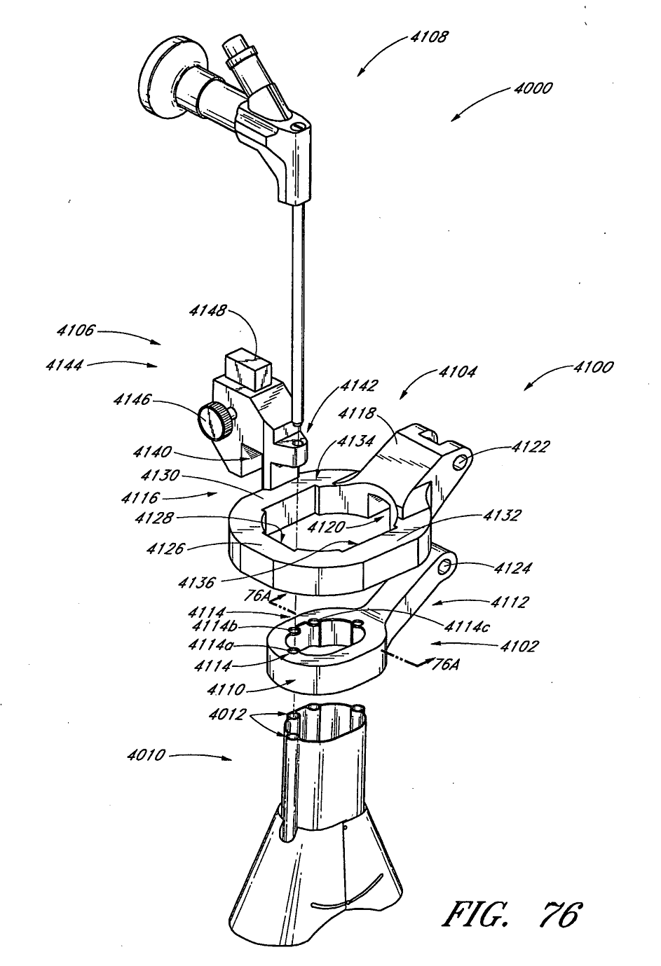

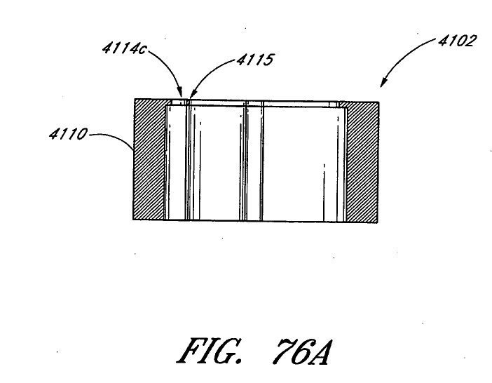

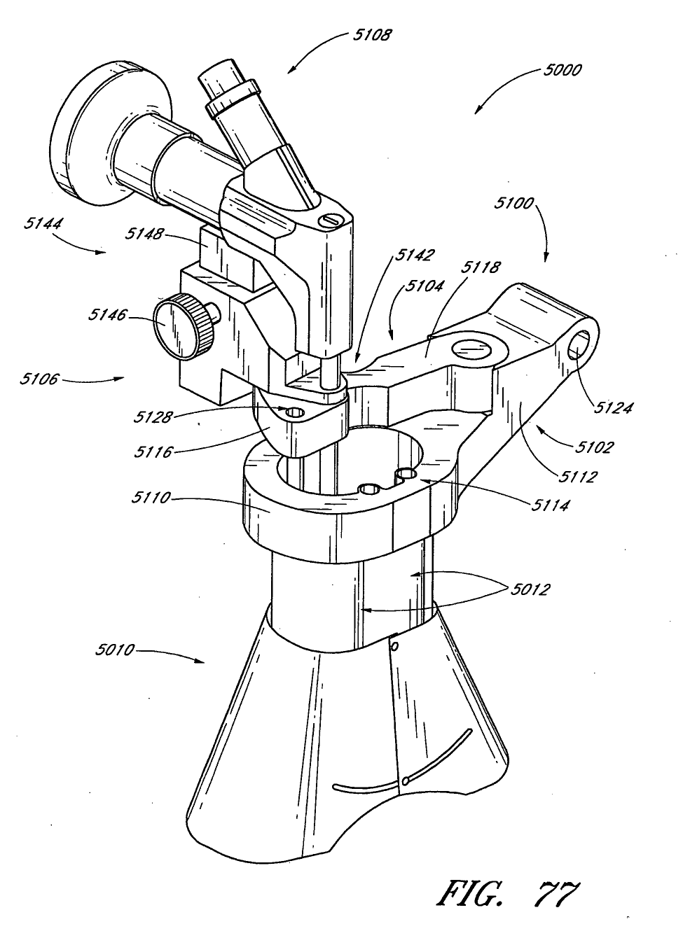

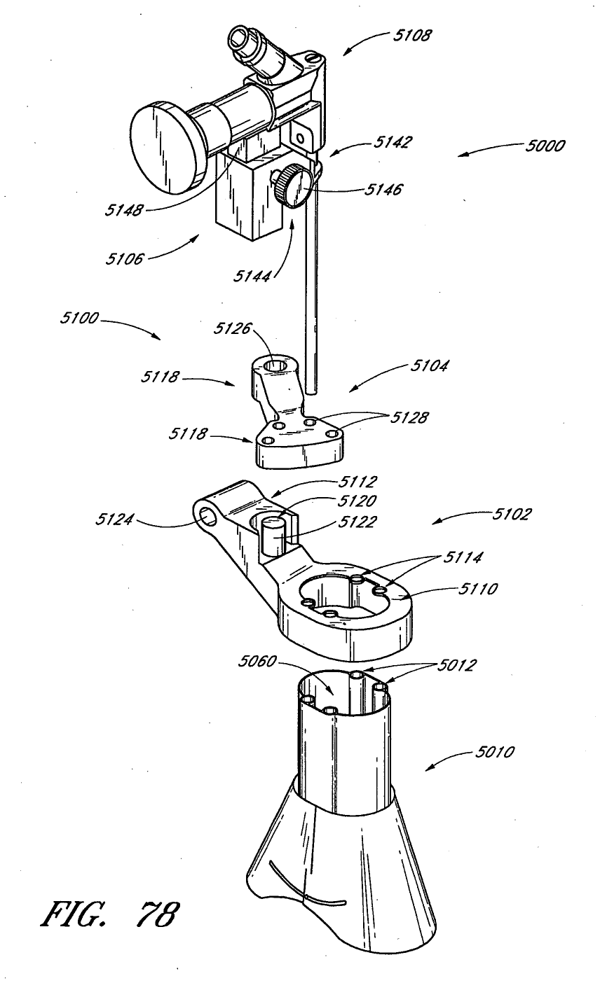

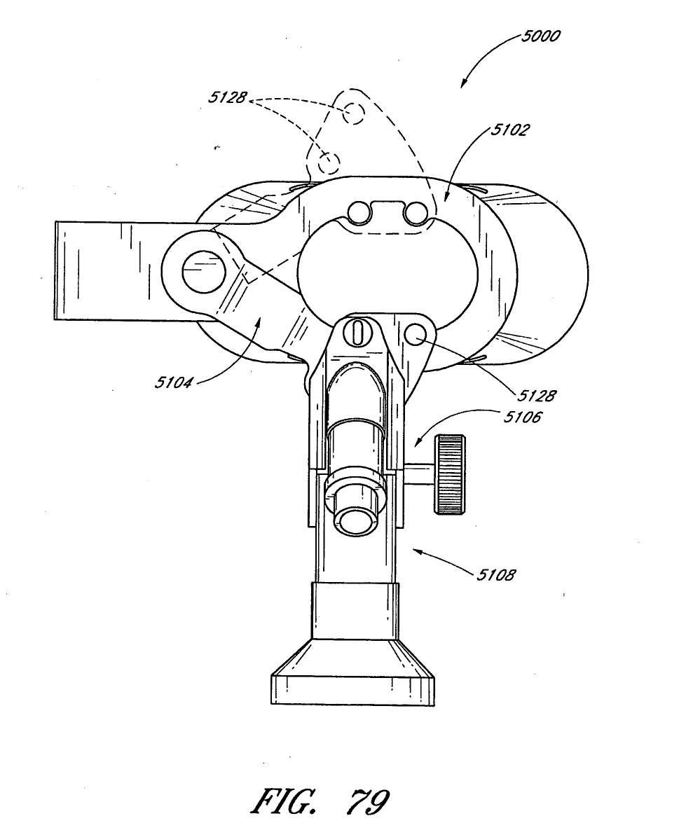

The elongate body 102 of the access device 100 has a first location 118 distal of a second location 120. The elongate body 102 preferably is capable of having a configuration when inserted within the patient wherein the cross-sectional area of the passage 104 at the first location 118 is greater than the cross-sectional area of the passage 104 at the second location 120. The passage 104 preferably is capable of having an oblong shaped cross section between the second location 120 and the proximal end 106. In some embodiments the passage 104 preferably is capable of having a generally elliptical cross section between the second location 120 and the proximal end 106. Additionally, the passage 104 preferably is capable of having a non-circular cross section between the second location 120 and the proximal end 106. Additionally, in some embodiments, the cross section of the passage 104 can be symmetrical about a first axis and a second axis, the first axis being generally normal to the second axis. Other embodiments having an oblong cross-section are discussed below in connection with FIGS. 67-79.

In another embodiment, an access device comprises an elongate body defining a passage and having a proximal end and a distal end. The elongate body can be a unitary structure and can have a generally uniform cross section from the proximal end to the distal end. In one embodiment, the elongate body preferably has an oblong or generally oval shaped cross section along the entire length of the elongate body. The passage can have a generally elliptical cross section between the proximal end and the distal end. The elongate body preferably has a relatively fixed cross-sectional area along its entire length. In one embodiment, the elongate body is capable of having a configuration when inserted within the patient wherein the cross-sectional area of the passage at a first location is equal to the cross-sectional area of the passage at a second location. The passage preferably is capable of having an oblong shaped cross section between the first and second locations. The cross section of the passage can be of any suitable oblong shape and the elongate body can be any desired size. Preferably, the elongate body is sized to provide sufficient space for inserting multiple surgical instruments sequentially or simultaneously through the elongate body to the surgical location.

In one embodiment, the access device has a uniform, generally oblong shaped cross section and is sized or configured to approach, dock on, or provide access to, anatomical structures. The access device preferably is configured to approach the spine from a posterior position or from a postero-lateral position. A distal portion of the access device can be configured to dock on, or provide access to, posterior portions of the spine for performing spinal procedures, such as, for example, fixation, fusion, or any other procedure described herein. In one embodiment, the distal portion of the access device has a uniform, generally oblong shaped cross section and is configured to dock on, or provide access to, generally posterior spinal structures. Generally posterior spinal structures can include, for example, one or more of the transverse process, the superior articular process, the inferior articular process, and the spinous process. In some embodiments, the access device can have a contoured distal end to facilitate docking on one or more of the posterior spinal structures. Accordingly, in one embodiment, the access device has a uniform, generally oblong shaped cross section with a distal end sized, configured, or contoured to approach, dock on, or provide access to, spinal structures from a posterior or postero-lateral position.

Further details and features pertaining to access devices and systems are described in U.S. patent application Ser. No. 09/772,605, filed Jan. 30, 2001, application Ser. No. 09/906,463, filed Jul. 16, 2001, application Ser. No. 10/361,887, filed Feb. 10, 2003, application Ser. No. 10/280,489, filed Oct. 25, 2002, and application Ser. No. 10/678,744 filed Oct. 2, 2003, which are incorporated by reference in their entireties herein.

2. Dilators and Expander Devices

According to one application or procedure, an early stage involves determining a point in the skin of the patient at which to insert the access device 20. The access point preferably corresponds to a posterior-lateral aspect of the spine. Manual palpation and Anterior-Posterior (AP) fluoroscopy may be used to determine preferred or optimal locations for forming an incision in the skin of the patient. In one application, the access device 20 preferably is placed midway (in the cephcaudal direction) between the L4 through S1 vertebrae, centrally about 4-7 cm from the midline of the spine.

After the above-described location is determined, an incision is made at the location. A guide wire (not shown) is introduced under fluoroscopic guidance through the skin, fascia, and muscle to the approximate surgical site. A series of dilators is used to sequentially expand the incision to the desired width, about 23 mm in one procedure, preferably minimizing damage to the structure of surrounding tissue and muscles. A first dilator can be placed over the guide wire to expand the opening. The guide wire may then be removed. A second dilator, slightly larger than the first dilator, is placed over the first dilator to expand the opening further. Once the second dilator is in place, the first dilator may be removed. This process of (1) introducing a next-larger-sized dilator coaxially over the previous dilator and (2) optionally removing the previous dilator(s) when the next-larger-sized dilator is in place continues until an opening of the desired size is created in the skin, muscle, and subcutaneous tissue. According to one application, the desired opening size is about 23 mm. (Other dimensions of the opening, e.g., about 20 mm, about 27 mm, about 30 mm, etc., are also useful with this apparatus in connection with spinal surgery, and still other dimensions are contemplated.)

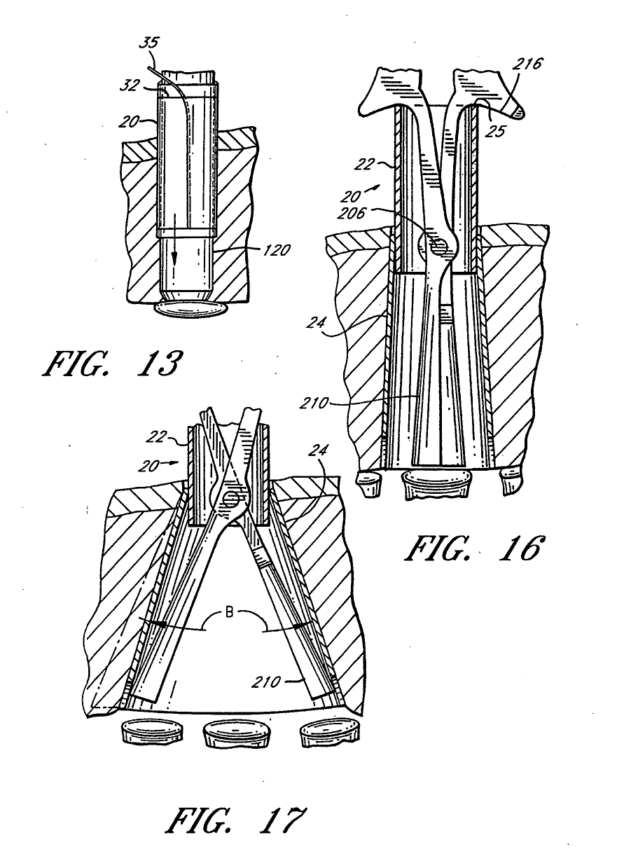

FIG. 13 shows that following placement of a dilator 120, which is the largest dilator in the above-described dilation process, the access device 20 is introduced in its reduced profile configuration and positioned over the dilator 120. The dilator 120 is subsequently removed from the patient, and the access device 20 remains in position.

Once positioned in the patient, the access device 20 may be enlarged to provide a passage for the insertion of various surgical instruments and to provide an enlarged space for performing the procedures described herein. As described above, the access device may achieve the enlargement in several ways. In one embodiment, a distal portion of the access device may be enlarged, and a proximal portion may maintain a constant diameter. The relative lengths of the proximal portion 22 and the skirt portion 24 may be adjusted to vary the overall expansion of the access device 20. Alternatively, such expansion may extend along the entire length of the access device 20. In one application, the access device 20 may be expanded by removing a suture 35 and tearing the outer sleeve 32 surrounding the access device 20, and subsequently allowing the skirt portion 24 to resiliently expand towards its fully expanded configuration as (illustrated in FIG. 4) to create an enlarged surgical space from the L4 to the S1 vertebrae. The resisting force exerted on the skirt portion 24 may result in the skirt portion 24 assuming the intermediate configuration illustrated in FIG. 3. Under many circumstances, the space created by the skirt portion 24 in the intermediate configuration is a sufficiently large working space to perform the procedure described herein. Once the skirt portion 24 has expanded, the rigidity and resilient characteristics of the skirt portion 24 preferably allow the access device 20 to resist closing to the reduced profile configuration of FIG. 2 and to at least temporarily resist being expelled from the incision. These characteristics create a stable configuration for the access device 20 to remain in position in the body, supported by the surrounding tissue. It is understood that additional support may be needed, especially if an endoscope is added.

According to one embodiment of a procedure, the access device 20 may be further enlarged at the skirt portion 24 using an expander apparatus to create a surgical access space. An expander apparatus useful for enlarging the access device has a reduced profile configuration and an enlarged configuration. The expander apparatus is inserted into the access device in the reduced profile configuration, and subsequently expanded to the enlarged configuration. The expansion of the expander apparatus also causes the access device to be expanded to the enlarged configuration. In some embodiments, the expander apparatus may increase the diameter of the access device along substantially its entire length in a generally conical configuration. In other embodiments, the expander apparatus expands only a distal portion of the access device, allowing a proximal portion to maintain a relatively constant diameter.

In addition to expanding the access device, in some embodiments the expander apparatus may also be used to position the distal portion of the access device at the desired location for the surgical procedure. The expander can engage an interior wall of the access device to move the access device to the desired location. For embodiments in which the distal portion of the access device is relatively movable with respect to the proximal portion, the expander apparatus is useful to position the distal portion without substantially disturbing the proximal portion.

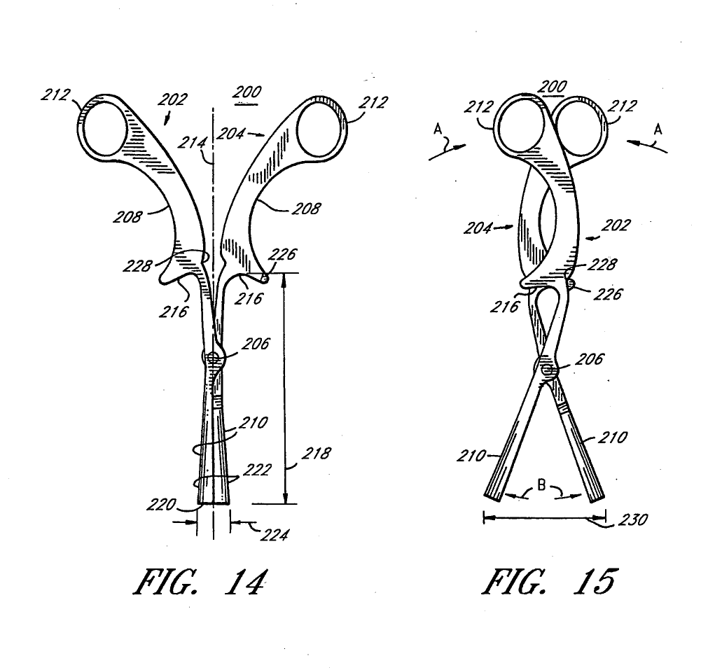

In some procedures, an expander apparatus is used to further expand the skirt portion 24 towards the enlarged configuration (illustrated in FIG. 4). The expander apparatus is inserted into the access device, and typically has two or more members that are movable to engage the interior wall of the skirt portion 24 and apply a force sufficient to further expand the skirt portion 24. FIGS. 14 and 15 show one embodiment of an expander apparatus 200 that has a first component 202 and a second component 204. The first component 202 and the second component 204 of the expander apparatus 200 are arranged in a tongs-like configuration and are pivotable about a pin 206. The first and second components 202 and 204 can be constructed of steel having a thickness of about 9.7 mm. Each of the first and second components 202 and 204 has a proximal handle portion 208 and a distal expander portion 210. Each proximal handle portion 208 has a finger grip 212 that may extend transversely from an axis, e.g., a longitudinal axis 214, of the apparatus 200. The proximal handle portion 208 may further include a stop element, such as flange 216, that extends transversely from the longitudinal axis 214. The flange 216 preferably is dimensioned to engage the proximal end 25 of the access device 20 when the apparatus 200 is inserted a predetermined depth. This arrangement provides a visual and tactile indication of the proper depth for inserting the expander apparatus 200. In one embodiment, a dimension 218 from the flange 216 to the distal tip 220 is about 106 mm. The dimension 218 is determined by the length of the access device 20, which in turn is a function of the depth of the body structures beneath the skin surface at which the surgical procedure is to be performed. The distal portions 210 are each provided with an outer surface 222 for engaging the inside wall of the skirt portion 24. The outer surface 222 is a frusto-conical surface in one embodiment. The expander apparatus 200 has an unexpanded distal width 224 at the distal tip 220 that is about 18.5 mm in one embodiment.

In use, the finger grips 212 are approximated towards one another, as indicated by arrows A in FIG. 15, which causes the distal portions 210 to move to the enlarged configuration, as indicated by arrows B. The components 202 and 204 are also provided with a cooperating tab 226 and shoulder portion 228 which are configured for mutual engagement when the distal portions 210 are in the expanded configuration. In the illustrated embodiment, the expander apparatus 200 has an expanded distal width 230 that extends between the distal portions 210. The expanded distal width 230 can be about 65 mm or less, about as large as 83 mm or less, or any other suitable width. The tab 226 and shoulder portion 228 together limit the expansion of the expander apparatus 200 to prevent expansion of the skirt portion 24 of the access device 20 beyond its designed dimension, and to minimize trauma to the underlying tissue. Further features related to the expander apparatus are described in U.S. Pat. No. 6,652,553, issued Nov. 25, 2003, which is incorporated by reference in its entirety herein.

When the access device 20 is inserted into the patient and the outer sleeve 32 is removed, the skirt portion 24 expands to a point where the outward resilient expansion of the skirt portion 24 is balanced by the force of the surrounding tissue. The surgical space defined by the access device 20 may be sufficient to perform any of a number of surgical procedures or combination of surgical procedures described herein. However, if it is desired to expand the access device 20 further, the expander apparatus 200, or a similar device, may be inserted into the access device 20 in the reduced profile configuration until the shoulder portions 216 are in approximation with the proximal end 25 of the skirt portion 24 of the access device 20, as shown in FIG. 16.

FIG. 16 shows the expander apparatus 200 inserted in the access device 20 in the reduced profiled configuration. Expansion of the expander apparatus 200 is achieved by approximating the handle portions 212 (not shown in FIG. 16), which causes the distal portions 210 of the expander apparatus 200 to move to a spaced apart configuration. As the distal portions 210 move apart and contact the inner wall of the skirt portion 24, the rivet 44 is allowed to slide within the slots 46 and 48 of the skirt portion 24, thus permitting the skirt portion 24 to expand. When the distal portions 210 reach the maximum expansion of the skirt portion 24 (illustrated by a dashed line in FIG. 17), the tab 226 and shoulder portion 228 of the expander apparatus 200 come into engagement to prevent further expansion of the tongs-like portions (as illustrated in FIG. 15). Alternatively, the access device 20 may be expanded with another device that can selectively have a reduced profile configuration and an expanded configuration, e.g., a balloon or similar device.

An optional step in the procedure is to adjust the location of the distal portion of the access device 20 relative to the body structures to be operated on. For example, the expander apparatus 200 may also be used to engage the inner wall of the skirt portion 24 of the access device 20 in order to move the skirt portion 24 of the access device 20 to the desired location. For an embodiment in which the skirt portion 24 of the access device 20 is relatively movable relative to the proximal portion, e.g. by use of the rivet 30, the expander apparatus 200 is useful to position the skirt portion 24 without substantially disturbing the proximal portion 22 or the tissues closer to the skin surface of the patient. As will be described below, the ability to move the distal end portion, e.g., the skirt portion 24, without disturbing the proximal portion is especially beneficial when an additional apparatus is mounted relative to the proximal portion of the access device, as described below.

B. Systems and Devices for Stabilization and Visualization

Some procedures can be conducted through the access device 20 without any additional peripheral components being connected thereto. In other procedures it may be beneficial to provide at least one of a support device and a viewing element. As discussed more fully below, support devices can be advantageously employed to provide support to peripheral equipment and to surgical tools of various types. Various embodiments of support devices and viewing elements are discussed herein below.

1. Support Devices

One type of support device that can be coupled with the access device 20 is a device that supports a viewing element. In one embodiment, an endoscope mount platform 300 and indexing arm 400 support an endoscope 500 on the proximal end 25 of the access device 20 for remotely viewing the surgical procedure, as illustrated in FIGS. 18-21. The endoscope mount platform 300 may also provide several other functions during the surgical procedure. The endoscope mount platform 300 preferably includes a base 302 that extends laterally from a central opening 304 in a generally ring-shaped configuration. In one application, the physician views the procedure primarily by observing a monitor, when inserting surgical instruments into the central opening 304. The base 302 advantageously enables the physician by providing a visual indicator (in that it may be observable in the physician's peripheral vision) as well as tactile feedback as instruments are lowered towards the central opening 304 and into the access device 20.

The endoscope mount platform 300 preferably has a guide portion 306 at a location off-set from the central opening 304 that extends substantially parallel to a longitudinal axis 308. The base 302 can be molded as one piece with the guide portion 306. The base 302 and guide portion 306 may be constructed with a suitable polymer, such as, for example, polyetheretherketone (PEEK).

The guide portion 306 includes a first upright member 310 that extends upward from the base 302 and a second upright member 312 that extends upward from the base 302. In one embodiment, the upright members 310, 312 each have a respective vertical grooves 314 and 315 that can slidably receive an endoscopic mount assembly 318.

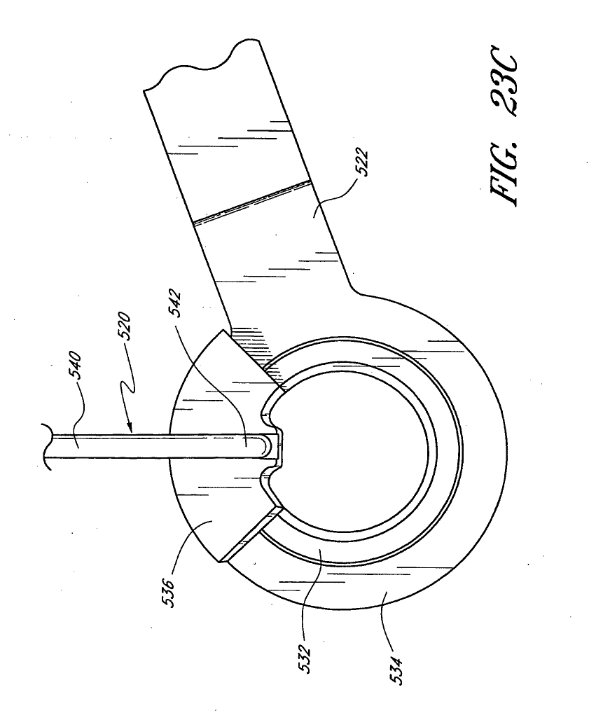

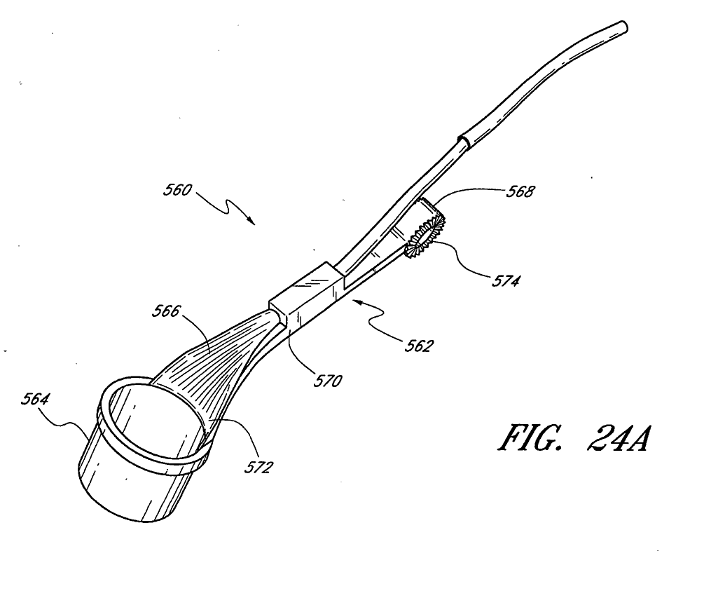

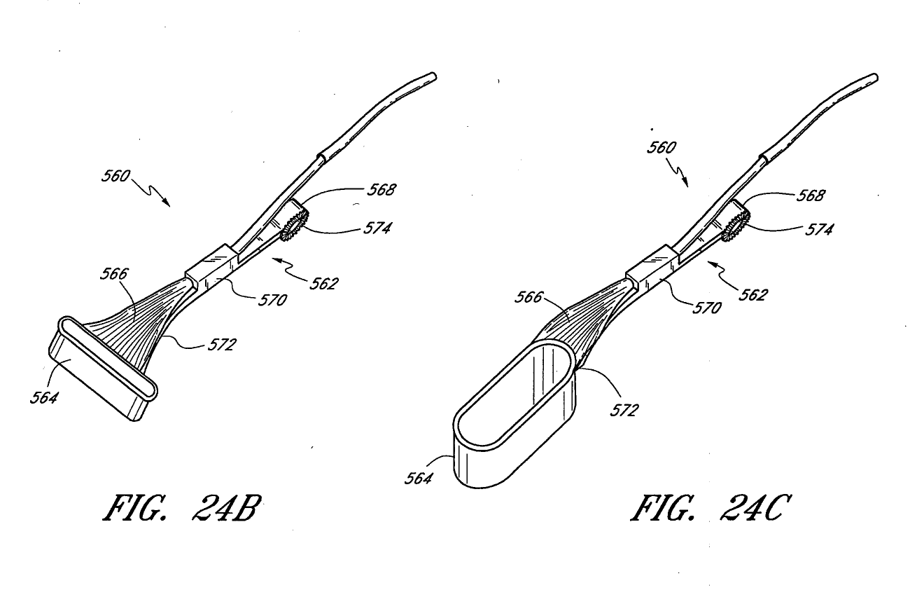

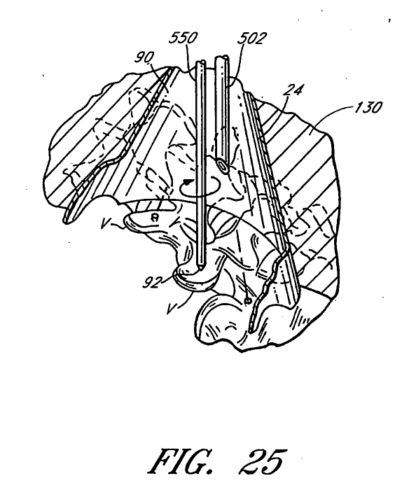

The endoscope 500 (not shown in FIG. 18) can be movably mounted to the endoscope mount platform 300 with the endoscope mount assembly 318 in one embodiment. The endoscope mount assembly 318 includes an endoscope mount 320 and a saddle unit 322. The saddle unit 322 is slidably mounted within the grooves 314 and 315 in the upright members 310 and 312. The endoscope mount 320 receives the endoscope 500 through a bore 326 which passes through the endoscope mount 320. Part of the endoscope 500 may extend through the access device 20 substantially parallel to longitudinal axis 308 into the patient's body 130, as shown in FIG. 25.

The endoscope mount 320 is removably positioned in a recess 328 defined in the substantially “U”-shaped saddle unit 322. In one embodiment, the saddle unit 322 is selectively movable in a direction parallel to the longitudinal axis 308 in order to position the endoscope 500 at the desired height within the access device 20. The movement of the endoscope 500 by way of the saddle unit 322 also advantageously enables the physician to increase visualization of a particular portion of the surgical space defined by the access device, e.g., by way of a zoom feature, as required for a given procedure or a step of a procedure.

In one embodiment, an elevation adjustment mechanism 340, which may be a screw mechanism, is positioned on the base 302 between the upright members 310 and 312. The elevation adjustment mechanism 340 can be used to selectively move a viewing element, e.g., the endoscope 500 by way of the saddle unit 322. In one embodiment, the elevation adjustment mechanism 340 comprises a thumb wheel 342 and a spindle 344. The thumb wheel 343 is rotatably mounted in a bore in the base 302. The thumb wheel 342 has an external thread 346 received in a cooperating thread in the base 302. The spindle 344 is mounted for movement substantially parallel to the central axis 308. The spindle 344 preferably has a first end received in a rectangular opening in the saddle unit 322, which inhibits rotational movement of the spindle 344. The second end of the spindle 344 has an external thread that cooperates with an internal thread formed in a bore within the thumb wheel 342. Rotation of the thumb wheel 342 relative to the spindle 344, causes relative axial movement of the spindle unit 344 along with the saddle unit 322. Further details and features related to endoscope mount platforms are described in U.S. Pat. No. 6,361,488, issued Mar. 26, 2002; U.S. Pat. No. 6,530,880, issued Mar. 11, 2003, and U.S. patent application Ser. No. 09/940,402, filed Aug. 27, 2001, published as Publication No. 2003/0040656 on Feb. 27, 2003, which are incorporated by reference in their entireties herein.

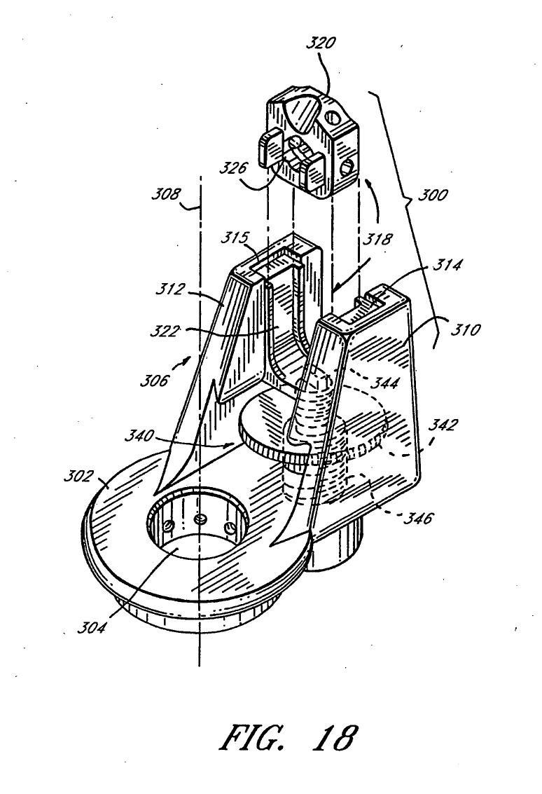

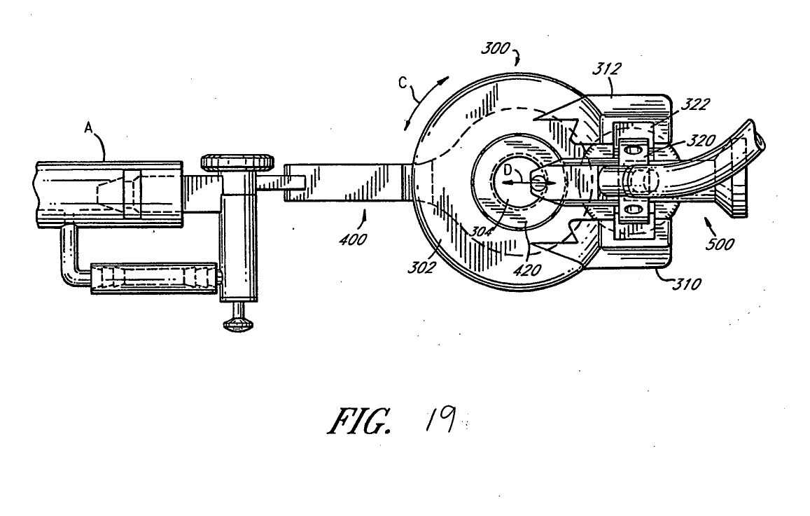

FIGS. 19-21 show that the endoscope mount platform 300 is mountable to the support arm 400 in one embodiment. The support arm 400, in turn, preferably is mountable to a mechanical support, such as mechanical support arm A, discussed above in connection with FIG. 1. The support arm 400 preferably rests on, or is otherwise coupled to, the proximal end 25 of the access device 20. In one embodiment, the support arm 400 is coupled with an indexing collar 420, which is configured to be received in the central opening 304 of the base 302 of endoscope mount platform 300. The indexing collar 420 is substantially toroidal in section and has an outer peripheral wall surface 422, an inner wall surface 424, and a wall thickness 426 that is the distance between the wall surfaces 422, 424. The indexing collar 420 further includes a flange 428, which supports the indexing collar 420 on the support arm 400.

In one embodiment, a plurality of collars 420 may be provided to make the surgical system 10 modular in that different access devices 20 may be used with a single endoscope mount platform 300. For example, access devices 20 of different dimensions may be supported by providing indexing collars 420 to accommodate each access device size while using a single endoscope mount platform 300. The central opening 304 of the endoscope mount platform 300 can have a constant dimension, e.g., a diameter of about 32.6 mm. An appropriate indexing collar 420 is selected, e.g., one that is appropriately sized to support a selected access device 20. Thus, the outer wall 422 and the outer diameter 430 are unchanged between different indexing collars 420, although the inner wall 424 and the inner diameter 432 vary to accommodate differently sized access devices 20.

The indexing collar 420 can be mounted to the proximal portion of the access device 20 to allow angular movement of the endoscope mount platform 300 with respect thereto about the longitudinal axis 308 (as indicated by an arrow C in FIG. 19). The outer wall 422 of the index collar 420 includes a plurality of hemispherical recesses 450 that can receive one or more ball plungers 350 on the endoscope mount platform 300 (indicated in dashed line). This arrangement permits the endoscope mount platform 300, along with the endoscope 500, to be fixed in a plurality of discrete angular positions.

Further details and features related to support arms and indexing collars are described in U.S. Pat. No. 6,361,488, issued Mar. 26, 2002, U.S. Pat. No. 6,530,880 issued Mar. 11, 2003, and application Ser. No. 09/940,402 filed Aug. 27, 2001, published as Publication No. 2003/0040656 on Feb. 27, 2003, which are incorporated by reference in their entireties herein.

2. Viewing Elements

As discussed above, a variety of viewing elements and visualization techniques are embodied in variations of the surgical system 10. One viewing element that is provided in one embodiment is an endoscope.

FIG. 22 shows one embodiment of the endoscope 500 that has an elongated configuration that extends into the access device 20 in order to enable viewing of the surgical site. In particular, the endoscope 500 has an elongated rod portion 502 and a body portion 504. The rod portion 502 extends generally perpendicularly from the body portion 504. In one embodiment, the rod portion 502 of endoscope 500 has a diameter of about 4 mm and a length of about 106 mm. Body portion 504 may define a tubular portion 506 configured to be slidably received in the bore 326 of endoscope mount 320 as indicated by an arrow D. The slidable mounting of the endoscope 500 on the endoscope mount platform 300 permits the endoscope 500 to adjust to access device configurations that have different diameters. Additional mobility of the endoscope 500 in viewing the surgical site may be provided by rotating the endoscope mount platform 300 about the central axis 308 (as indicated by arrow C in FIG. 19).

The rod portion 502 supports an optical portion (not shown) at a distal end 508 thereof. In one embodiment, the rod portion 502 defines a field of view of about 105 degrees and a direction of view 511 of about 25-30 degrees. An eyepiece 512 preferably is positioned at an end portion of the body portion 504. A suitable camera (not shown) preferably is attached to the endoscope 500 adjacent the eyepiece 512 with a standard coupler unit. A light post 510 can supply illumination to the surgical site at the distal end portion 508. A preferred camera for use in the system and procedures described herein is a three chip unit that provides greater resolution to the viewed image than a single chip device.

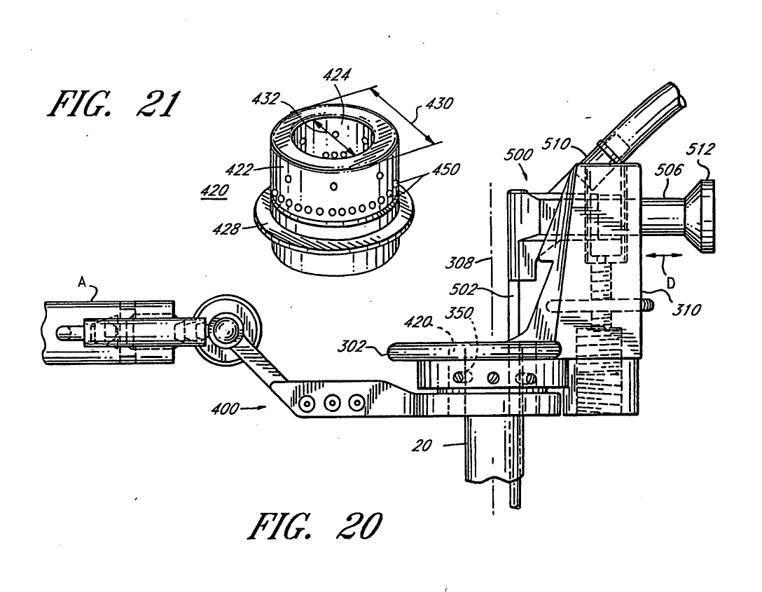

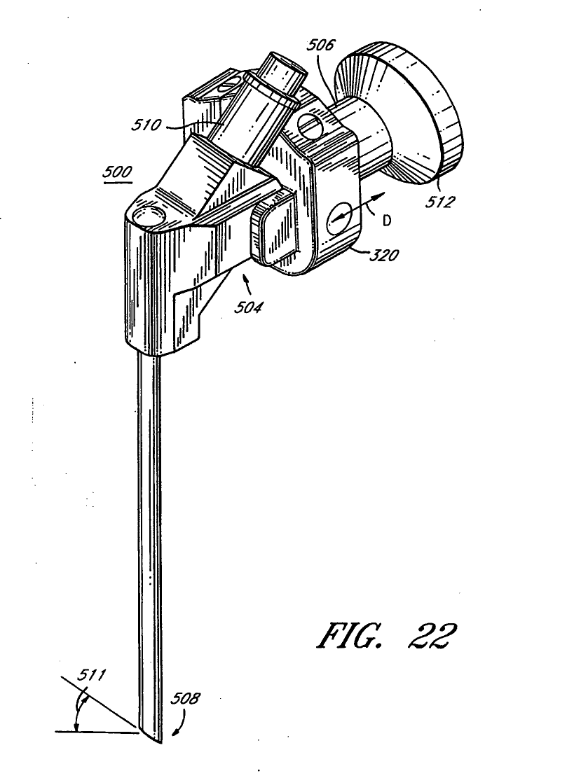

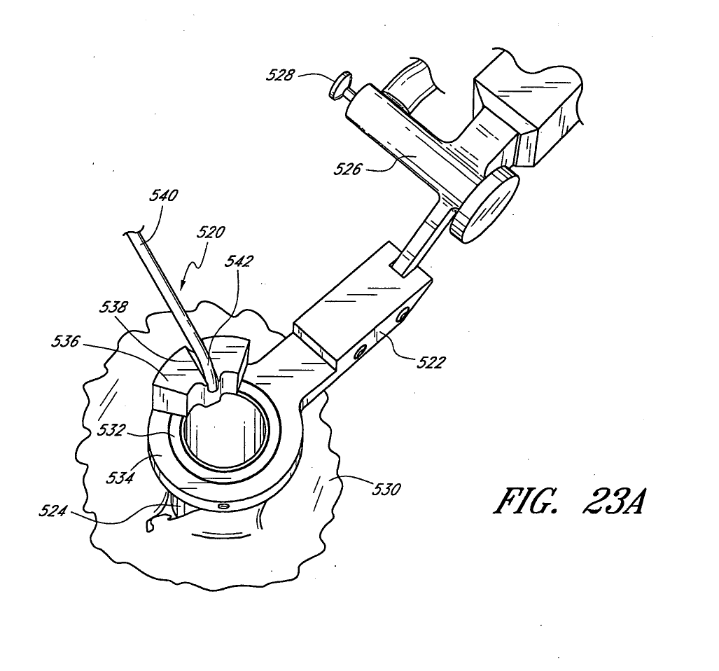

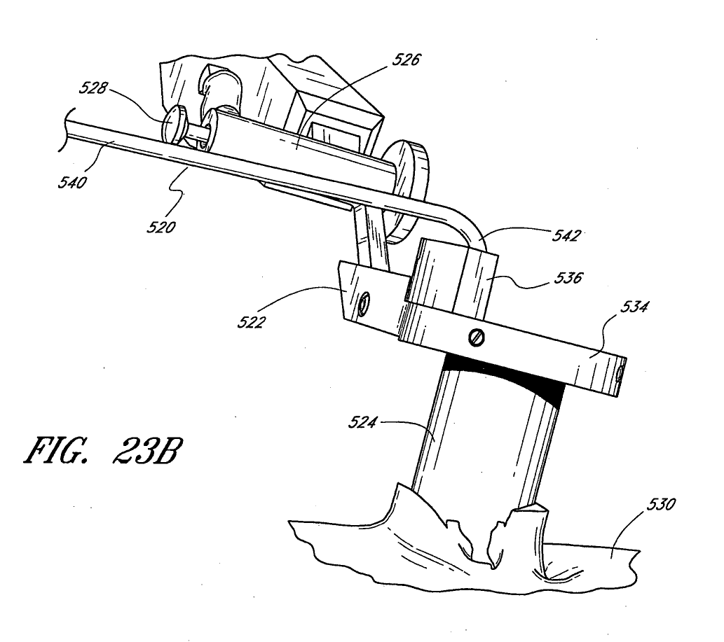

FIGS. 23A, 23B, 23C, 24A, 24B, and 24C illustrate other embodiments of support devices and viewing elements. FIGS. 23A, 23B, and 23C illustrate one embodiment of a lighting element 520 coupled with a support arm 522 compatible with an access device 524 having a proximal portion with a generally circular cross section. In other embodiments, support arms can be configured to be coupled with access devices having proximal portions with generally oblong or oval cross sections.

The support arm 522 preferably is coupled with the access device 524 to provide support for the access device 524 during a procedure. As shown in FIGS. 23A, 23B, and 23C, the support arm 522 comprises a pneumatic element 526 for maintaining the support arm 522 in a desired position. Depressing a button 528 coupled with a valve of the pneumatic element 526 releases pressure and allows the support arm 522 and access device 524 to be moved relative the patient 530. Releasing the button 528 of the pneumatic element 526 increases pressure and maintains the access device 524 and support arm 522 in the desired position. The support arm 522, as shown, is configured for use with a mechanical arm using a suction, or a vacuum to maintain the access device in a desired location. One of skill in the art will recognize that various other support arms and mechanical arms can be used. For example, commercially available mechanical arms having clamping mechanisms can be used as well as suction or pressure based arms.

The support arm 522 can comprise an inner ring portion 532 and an outer ring portion 534 for surrounding the access device 524 at its proximal end. In the illustrated embodiment, the inner and outer ring portions 532, 534 are fixed relative each other. In other embodiments the inner and outer ring portions 532, 534 can move relative each other. The support arm 522 preferably comprises a lighting element support portion 536. In the illustrated embodiment, the lighting element support portion 536 extends above upper surfaces of the inner and outer ring portions 532, 534. The lighting element support portion 536 can extend from the inner ring portion 532, the outer ring portion 534, or both. The lighting element support portion 536 can have a notch or groove 538 for receiving and supporting the lighting element 520. Additionally, the lighting element support portion 536 can have one or more prongs extending at least partially over the lighting element 520 to hold it in place.

In the illustrated embodiment, the lighting element 520 has an elongated proximal portion 540 and a curved distal portion 542. The proximal portion 540 of the lighting element 520 preferably is coupled with a light source (not shown). The curved distal portion of the lighting element 520 in one embodiment extends only a short distance into the access device and is configured to direct light from the light source down into the access device 524. In another embodiment, the lighting element 520 can be provided such that it does not extend into the access device. In such an embodiment, the right portions 532 and 534 only partially surround the proximal end of the access device 524. Providing a lighting element 520 for use with the access device 524 preferably allows a user to see down into the access device 524 to view a surgical location. Accordingly, use of a lighting element 520 in some cases, enables the user to perform a procedure, in whole or in part, without the use of an endoscope. In one embodiment, the lighting element 520 enables a surgeon to perform the procedure with the use of microscopes or loupes.

FIGS. 24A, 24B, and 24C illustrate other embodiments of visualization elements. As shown in FIG. 24A, a lighting element 560 comprises a support member 562, an access device insert 564, and fiber optic elements 566. The support member 562 has a proximal end 568, a central portion 570, and a distal end 572. The proximal end 568 preferably has a coupling portion 574 for coupling the support member 562 to a support arm or other support system (not shown). The central portion 570 preferably is coupled with the fiber optic elements 566 to provide support there to. The distal end 572 preferably is coupled with the access device insert 564.

In the illustrated embodiment, the access device insert 564 is configured to be inserted in an access device having a proximal portion with a generally circular cross section. The access device insert 564 is coupled with the fiber optic elements 566. The fiber optic elements 566 extend down into the access device insert 564 so that the ends of the fiber optic elements 566 can direct light down inside an access device along side portions there of.

FIGS. 24B and 24C illustrate other embodiments of visualization elements similar to the embodiment described with reference to FIG. 24A. In the illustrated embodiments, the access device inserts 564 are configured to be inserted into access devices having proximal portions with generally oblong, or oval, cross sections. As shown in FIG. 24B, the access device insert 564 has a generally oblong or oval shaped cross section. The access device insert 564 is coupled with the fiber optic elements 566 along a longer side surface of the access device insert 564. As shown in FIG. 24C, the access device insert 564 has a generally oblong or oval shaped cross section. The access device insert 564 is coupled with the fiber optic elements 566 along a shorter side surface of the access device insert 564. Use of an illumination element with an expandable access device having an oblong shaped proximal section, in some cases, allows a doctor to perform procedures that would be difficult to perform using an endoscope. Increased visualization of the surgical location through the access device can simplify some procedures. For example, decompression of the contra-lateral side can be achieved more easily in some cases without the use of an endoscope.

C. Apparatuses and Methods for Performing Spinal Procedures

The surgical assembly 10 described above can be deployed to perform a wide variety of surgical procedures on the spine. In many cases, the procedures are facilitated by inserting the access device and configuring it to provide greater access to a surgical location, as discussed above and by mounting the support arm 400 and the endoscope mount platform 300 on the proximal portion, e.g., on the proximal end 25, of the access device 20 (FIGS. 1 and 22). As discussed above, visualization of the surgical location is enhanced by mounting a viewing element, such as the endoscope 500, on the endoscope mount platform 300. Having established increased access to and visualization of the surgical location, a number of procedures may be effectively performed.

Generally, the procedures involve inserting one or more surgical instruments into the access device 20 to manipulate or act on the body structures that are located at least partially within the operative space defined by the expanded portion of the access device 20. FIG. 25 shows that in one method, the skirt portion 24 of access device 20 at least partially defines a surgical site or operative space 90 in which the surgical procedures described herein may be performed. Depending upon the overlap of the skirt portion, the skirt portion may define a surface which is continuous about the perimeter or which is discontinuous, having one or more gaps where the material of the skirt portion does not overlap.

One procedure performable through the access device 20, described in greater detail below, is a two-level spinal fusion and fixation. Surgical instruments inserted into the access device may be used for debridement and decortication. In particular, the soft tissue, such as fat and muscle, covering the vertebrae may be removed in order to allow the physician to visually identify the various “landmarks,” or vertebral structures, which enable the physician to determine the location for attaching a fastener, such a fastener 600, discussed below, or other procedures, as will be described herein. Enabling visual identification of the vertebral structures enables the physician to perform the procedure while viewing the surgical area through the endoscope, microscope, loupes, or other viewing element, or in a conventional, open manner.

Tissue debridement and decortication of bone are completed using one or more of a debrider blades, a bipolar sheath, a high speed burr, and any other conventional manual instrument. The debrider blades are used to excise, remove and aspirate the soft tissue. The bipolar sheath is used to achieve hemostasis through spot and bulk tissue coagulation. Additional features of debrider blades and bipolar sheaths are described in U.S. Pat. No. 6,193,715, assigned to Medical Scientific, Inc., which is incorporated by reference in its entirety herein. The high speed burr and conventional manual instruments are also used to continue to expose the structure of the vertebrae.

1. Fixation Systems and Devices

Having increased visualization of the pertinent anatomical structure, various procedures may be carried out on the structures. In one procedure, one or more fasteners are attached to adjacent vertebrae V. As discussed in more detail below, the fasteners can be used to provide temporary or permanent fixation and to provide dynamic stabilization of the vertebrae V. These procedures may combined with other procedures, such as procedures employing other types of implant, e.g., procedures employing fusion devices, prosthetic disc components, or other suitable implants. In some procedures, fasteners are attached to the vertebrae before or after fusion devices are inserted between the vertebrae V. Fusion systems and devices are discussed further below.

In one application, the desired location and orientation of the fastener is determined before the fastener is applied to the vertebra. The desired location and orientation of the fastener may be determined in any suitable manner. For example, the pedicle entry point of the L5 vertebrae may be located by identifying visual landmarks alone or in combination with lateral and A/P fluoroscopy, as is known in the art. With continued reference to FIG. 25, an entry point 92 into the vertebra V is prepared. In procedure, the entry point 92 may be prepared with an awl 550. The entry point 92 corresponds to the pedicle in one procedure. The entry point 92 may be prepared in any suitable manner, e.g., employing a bone probe, a tap, and a sounder to create and verify the integrity of the prepared vertebra. The sounder, as is known in the art, determines whether the hole that is made is surrounded by bone on all sides, and can be used to confirm that there has been no perforation of the pedicle wall.

After the hole in the pedicle beneath the entry point 92 is prepared, a fastener may be advanced into the hole. Prior to advancing the fastener, or at any other point during the procedure, it may be desirable to adjust the location of the distal portion of the access device 20. The distal portion of the access device 20 may be adjusted by inserting the expander apparatus 200 into the access device 20, expanding the distal portions 210, and contacting the inner wall of the skirt portion 24 to move the skirt portion 24 to the desired location. This step may be performed while the endoscope 500 is positioned within the access device 20, and without substantially disturbing the location of the proximal portion of the access device 20 to which the endoscope mount platform 300 may be attached.

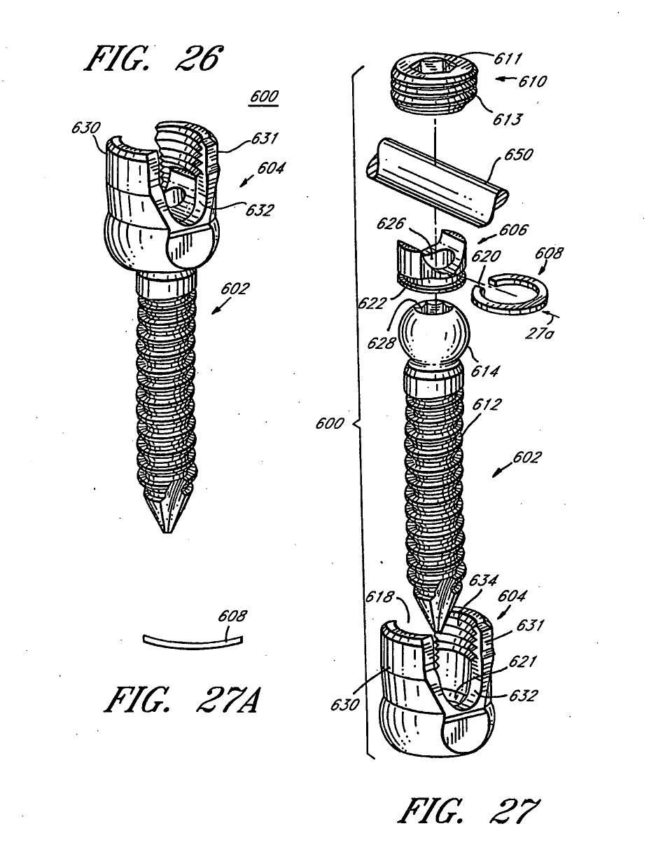

FIGS. 26-27 illustrate one embodiment of a fastener 600 that is particularly applicable in procedures involving fixation. The fastener 600 preferably includes a screw portion 602, a housing 604, a spacer member 606, a biasing member 608, and a clamping member, such as a cap screw 610. The screw portion 602 has a distal threaded portion 612 and a proximal, substantially spherical joint portion 614. The threaded portion 612 is inserted into the hole that extends away from the entry point 92 into the vertebrae, as will be described below. The substantially spherical joint portion 614 is received in a substantially annular, partly spherical recess 616 in the housing 604 in a ball and socket joint relationship (see also FIG. 29).

As illustrated in FIG. 27, the fastener 600 is assembled by inserting the screw portion 602 into a bore in a passage 618 in the housing 604 until the joint portion 614 engages the annular recess 616. The screw portion 602 is retained in the housing 604 by the spacer member 606 and by the biasing member 608. The biasing member 608 provides a biasing force to drive the spacer member 606 into frictional engagement with the joint portion 614 of the screw member 602 and the annular recess 616 of the housing 604. The biasing provided by the biasing member 602 frictionally maintains the relative positions of the housing 604 with respect to the screw portion 602. The biasing member 608 preferably is selected such that biasing force prevents unrestricted movement of the housing 604 relative to the screw portion 602. However, in some embodiments the biasing force is insufficient to resist the application of force by a physician to move the housing 604 relative to the screw portion 602. In other words, this biasing force is strong enough maintain the housing 604 stationary relative to the screw portion 602, but this force may be overcome by the physician to reorient the housing 604 with respect to the screw member 602, as will be described below.

In the illustrated embodiment, the biasing member 608 is a resilient ring having a gap 620, which permits the biasing member 608 to radially contract and expand. FIG. 27(a) illustrates that the biasing member 608 may have an arched shape, when viewed end-on. The arched shape of the spring member 608 provides the biasing force, as will be described below. The spacer member 606 and the biasing member 608 are inserted into the housing 604 by radially compressing the biasing member into an annular groove 622 in the spacer member 606. The spacer member 606 and the biasing member 608 are slid into the passage 618 until the distal surface of the spacer member 606 engages the joint portion 614 of the screw portion 602, and the biasing member 608 expands radially into the annular groove 622 in the housing 604. The annular groove 622 in the housing 604 has a dimension 623 that is smaller than the uncompressed height of the arched shape of the biasing member 608. When the biasing member 608 is inserted in the annular groove 620, the biasing member 608 is flattened against its normal bias, thereby exerting the biasing force to the spacer member 606. It is understood that similar biasing members, such as coiled springs, belleville washers, or the like may be used to supply the biasing force described herein.

The spacer member 606 is provided with a longitudinal bore 626, which provides access to a hexagonal recess 628 in the proximal end of the joint portion 614 of the screw member 602. The proximal portion of the housing 604 includes a pair of upright members 630 and 631 that are separated by substantially “U”-shaped grooves 632. A recess for receiving elongated member 650 is defined by the pair of grooves 632 between upright members 630 and 631. Elongated member 650 preferably is configured to be placed distally into the housing 604 in an orientation substantially transverse to the longitudinal axis of the housing 604, as will be described below. The inner walls of he upright members 630 and 631 are provided with threads 634 for attachment of the cap screw 610 by threads 613 therein.

Additional features of the fastener 600 are also described in U.S. patent application Ser. No. 10/075,668, filed Feb. 13, 2002, published as U.S. Application Publication No. 2003/0153911A1 on Aug. 14, 2003, and application Ser. No. 10/087,489, filed Mar. 1, 2002, published as U.S. Application Publication No. 2003/0167058A1 on Sep. 4, 2003, which are incorporated by reference in their entireties herein.

According to one application, the fastener 600 is inserted into the access device 20 and guided to the prepared hole at the entry point 92 in the vertebrae. The fastener 600 preferably is simultaneously supported and advanced into the hole so that the fastener 600 is secured in the in the hole beneath the entry point 92. In the illustrated embodiment the fastener 600 is supported and attached to the bone by an endoscopic screwdriver apparatus 660, illustrated in FIGS. 28-29. The screwdriver 660 includes a proximal handle portion 662 (illustrated in dashed line), an elongated body portion 664, and a distal tool portion 666.

The distal tool portion 666, as illustrated in greater detail in FIG. 29 includes a substantially hexagonal outer periphery that is received in the substantially hexagonal recess 628 in the joint portion 614 of the screw member 602. A spring member at the distal tool portion 666 releasably engages the hexagonal recess 628 of the screw member 602 to support the fastener 600 during insertion and tightening. In the illustrated embodiment, a spring member 672 is configured to engage the side wall of the recess 628. More particularly, a channel or a groove is provided in the tip portion 666 for receiving the spring member 672. The channel or groove includes a medial longitudinal notch portion 676, a proximal, angled channel portion 678, and a distal substantially transverse channel portion 680. The spring member 672 is preferably manufactured from stainless steel and has a medial portion 682, proximal portion 684, and a transverse distal portion 686. The medial portion 682 is partially received in the longitudinal notch portion 676. The proximal portion 684 preferably is angled with respect to the medial portion 682 and is fixedly received in the angled channel portion 678. The transverse distal portion 686 preferably is slidably received in the transverse channel 680. The medial portion 682 of the spring member 672 is partially exposed from the distal tip portion 666 and normally is biased in a transverse outward direction with respect to the longitudinal axis (indicated by arrow E), in order to supply bearing force against the wall of the recess 628. Alternatively, the distal tip portion of the screwdriver may be magnetized in order to hold the screw portion 602. Similarly, the distal tip portion may include a ball bearing or similar member which is normally biased in a radially outward direction to engage the interior wall of the recess 628 to secure the fastener 600 to the screwdriver distal tip 666. Other means may be provided for temporarily but securely coupling the fastener 600 with the screwdriver distal tip 666.

The insertion of the fastener 600 into the prepared hole that extends into the vertebrae from the entry point 92 may be achieved by insertion of screwdriver 660 into access device 20 (indicated by arrow G). This procedure may be visualized by the use of the endoscope 500 in conjunction with fluoroscopy, or by way of any other suitable viewing element. The screw portion 602 is threadedly advanced by the endoscopic screwdriver 660 into the prepared hole that extends beneath the entry point 92 (indicated by arrow H). The endoscopic screwdriver 660 is subsequently separated from the fastener 600, by applying a force in the proximal direction, and thereby releasing the distal tip portion 666 from the hexagonal recess 628 (e.g., causing the transverse distal portion 686 of the spring member 672 to slide within the transverse recess 680 against the bias, indicated by arrow F), and removing the screwdriver 660 from the access device 20. An alternative method may use a guidewire, which is fixed in the hole beneath the entry point 92, and a cannulated screw which has an internal lumen and is guided over the guidewire into the hole beneath the entry point 92. Where a guidewire system is used, the screwdriver also would be cannulated so that the screwdriver would fit over the guidewire.

For a two-level fixation, it may be necessary to prepare several holes and attach several fasteners 600. Preferably, the access device 20 is sized to provide simultaneous access to all vertebrae in which the surgical procedure is being performed. In some cases, however, additional enlargement or repositioning of the distal portion of the access device 20 may be helpful in providing sufficient access to the outer vertebrae, e.g., the L4 and S1 vertebrae. In the illustrated embodiment, the expander apparatus 200 may be repeatedly inserted into the access device 20 and expanded in order to further open or to position the skirt portion 24. In one procedure, additional fasteners are inserted in the L4 and S1 vertebrae in a similar fashion as the fastener 600 inserted into the L5 vertebra as described above. (When discussed individually or collectively, a fastener and/or its individual components will be referred to by the reference number, e.g., fastener 600, housing 604, and all fasteners 600. However, when several fasteners and/or their components are discussed in relation to one another, an alphabetic subscript will be used, e.g., fastener 600a is moved towards fastener 600b.)

In one application, after the fasteners 600 are advanced into the vertebrae, the housing portions 604 of the fasteners 600 are substantially aligned such that their upright portions 630 and 631 face upward, and the notches 632 are substantially aligned to receive the elongated member 650 therein. The frictional mounting of the housing 604 to the screw member 602, described above, allows the housing 604 to be temporarily positioned until a subsequent tightening step is performed, described below.

Positioning of the housing portions 604 may be performed by the use of an elongated surgical instrument capable of contacting and moving the housing portion to the desired orientation. One such instrument for positioning the housings 604 is a grasper apparatus 700, illustrated in FIG. 30. The grasper apparatus 700 includes a proximal handle portion 702, an elongated body portion 704, and distal nose portion 706. The distal nose portion 706 includes a pair of grasping jaws 708a and 708b, which are pivotable about pin 710 by actuation of the proximal handle portion 702. The grasping jaws 708a and 708b are illustrated in the closed position in FIG. 30. Pivoting the movable handle 714 towards stationary handle 712 causes longitudinal movement of actuator 716, which in turn pivots the jaw 708b towards an open position (illustrated in dashed line). The biasing members 718 and 720 are provided to return the handles 712 and 714 to the open position and bias the jaws 708a and 708b to the closed position.

In one application, the elongated member 650 is inserted into the access device 20. In one application, the elongated member 650 is manufactured from a biocompatible material and is sufficiently strong to maintain the position of the vertebrae, or other body structures, coupled by the elongate member 650 with little or no relative motion therebetween. In one embodiment, the elongated members 650 are manufactured from Titanium 6/4 or titanium alloy. The elongated member 650 also may be manufactured from stainless steel or any other suitable material. The transverse shape, width (e.g., radii), and lengths of the elongated members 650 are selected by the physician to provide the best fit for the positioning of the screw heads. Such selection may be performed by placing the elongated member 650 on the skin of the patient overlying the location of the fasteners and viewed fluoroscopically. For example, a 70 mm preformed rod having a 3.5″ bend radius may be selected for the spinal fixation.

In one application, the elongated member 650 is fixed to each of the fasteners 600, and more particularly, to the housings 604 of each fastener 600. The grasper apparatus 700, described above, is also particularly useful for inserting the elongated member 650 into the access device 20 and positioning it with respect to each housing 604. As illustrated in FIG. 30, the jaws 708a and 708b of the grasper apparatus 700 each has shaped (e.g., curved) contact portions 722a and 722b for contacting and holding the outer surface of the elongated member 650.

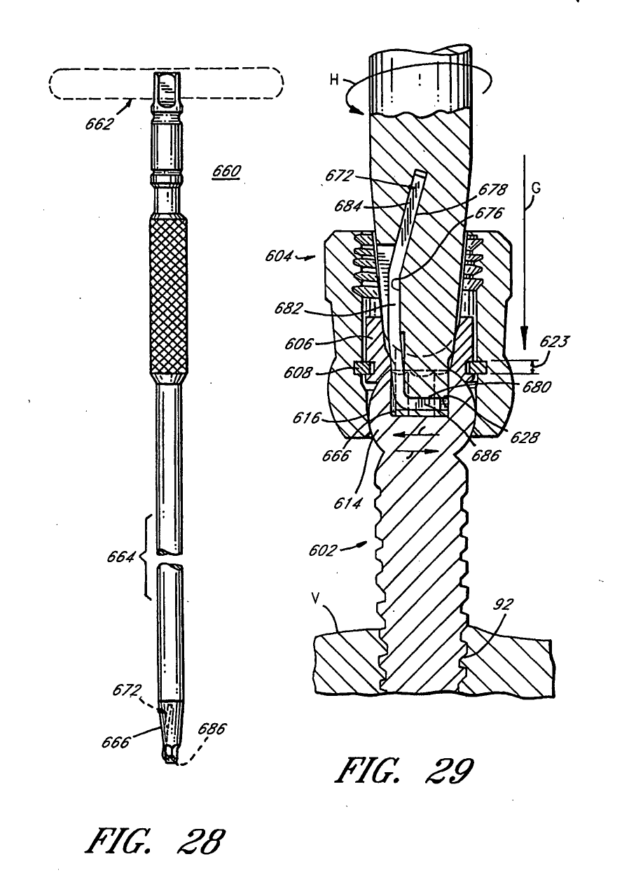

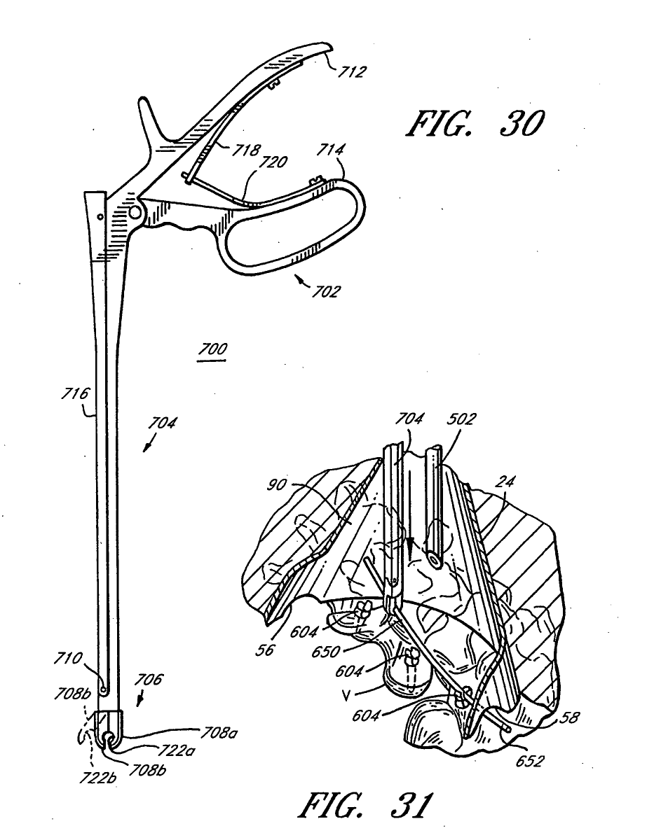

As illustrated in FIG. 31, the grasper apparatus 700 may be used to insert the elongated member 650 into the operative space 90 defined at least partially by the skirt portion 24 of the access device 20. In some embodiments, the cut-out portions 56 and 58 provided in the skirt portion 24 assist in the process of installing the elongated member 650 with respect to the housings 604. The cut-out portions 56 and 58 allow an end portion 652 of the elongated member 650 to extend beyond the operative space without raising or repositioning the skirt portion 24. The elongated member 650 is positioned within the recesses in each housing 604 defined by grooves 632 disposed between upright members 630 and 631. The elongated member 650 is positioned in an orientation substantially transverse to the longitudinal axis of each housing 604.

Further positioning of the elongated member 650 may be performed by guide apparatus 800, illustrated in FIG. 32. Guide apparatus 800 is useful in cooperation with an endoscopic screwdriver, such as endoscopic screwdriver 660 (illustrated in FIG. 28), in order to position the elongated member 650, and to introduce and tighten the cap screw 610, described above and illustrated in FIG. 27. Tightening of the cap screw 610 with respect to the housing 604 fixes the orientation of the housing 604 with respect to the screw portion 602 and fixes the position of the elongated member 650 with respect to the housings 604.

In the illustrated embodiment, the guide apparatus 800 has a proximal handle portion 802, an elongated body portion 804, and a distal tool portion 806. The elongated body portion 804 defines a central bore 808 (illustrated in dashed line) along its longitudinal axis 810. The central bore 808 is sized and configured to receive the endoscopic screwdriver 660 and cap screw 610 therethrough. In the exemplary embodiment, the diameter of the central bore 808 of the elongated body portion 804 is about 0.384-0.388 inches in diameter, and the external diameter of the endoscopic screwdriver 660 (FIG. 28) is about 0.25 inches. The proximal handle portion 802 extends transverse to the longitudinal axis 810, which allows the physician to adjust the guide apparatus 800 without interfering with the operation of the screwdriver 660.

The distal portion 806 of the apparatus includes several shaped cut out portions 814 which assist in positioning the elongated member 650. As illustrated in FIG. 33, the cut out portions 814 are sized and configured to engage the surface of elongated member 650 and move the elongated member 650 from an initial location (illustrated in dashed line) to a desired location. In the illustrated embodiment, the cut out portions 814 are semicircular, to match the round elongated member 650. However, other shaped cut out portions may be provided to match other shaped elongated members.

As illustrated in FIG. 34, the guide apparatus 800 is used in cooperation with the endoscopic screwdriver 660 to attach the cap screw 610. The distal end of the body portion 804 includes a pair of elongated openings 816. The openings 816 provide a window to enable the physician to endoscopically view the cap screw 610 retained at the distal tip 666 of the endoscopic screw driver 660. Fewer or more than two openings can be provided and the openings 816 need not be elongated.

The guide apparatus 800 and the endoscopic screwdriver 660 cooperate as follows in one application. The guide apparatus 800 is configured to be positioned in a surrounding configuration with the screwdriver 600. In the illustrated embodiment, the body portion 804 is configured for coaxial placement about the screwdriver 66Q in order to distribute the contact force of the guide apparatus 800 on the elongated member 650. The distal portion 806 of the guide apparatus 800 may bear down on the elongated member 650 to seat the elongated member 650 in the notches 632 in the housing 604. The “distributed” force of the guide apparatus 800 may contact the elongated member 650 on at least one or more locations. In addition, the diameter of central bore 808 is selected to be marginally larger than the exterior diameter of cap screw 610, such that the cap screw 610 may freely slide down the central bore 808, while maintaining the orientation shown in FIG. 34. This configuration allows the physician to have effective control of the placement of the cap screw 610 into the housing 604. The cap screw 610 is releasably attached to the endoscopic screwdriver 660 by means of spring member 672 engaged to the interior wall of hexagonal recess 611 as it is inserted within the bore 808 of the body portion 804 of guide apparatus 800. The cap screw 610 is attached to the housing 604 by engaging the threads 615 of the cap screw 610 with the threads 634 of the housing.

As illustrated in FIG. 35, tightening of the cap screw 610 fixes the assembly of the housing 604 with respect to the elongated member 650. In particular, the distal surface of the cap screw 610 provides a distal force against the elongated member 650, which in turn drives the spacer member 606 against the joint portion 614 of the screw portion 602, which is fixed with respect to the housing 604.

If locations of the vertebrae are considered acceptable by the physician, then the fixation procedure is substantially complete once the cap screws 610 have been attached to the respective housings 604, and tightened to provide a fixed structure as between the elongated member 650 and the various fasteners 600. However, if compression or distraction of the vertebrae with respect to one another is required additional apparatus would be used to shift the vertebrae prior to final tightening all of the cap screws 610.

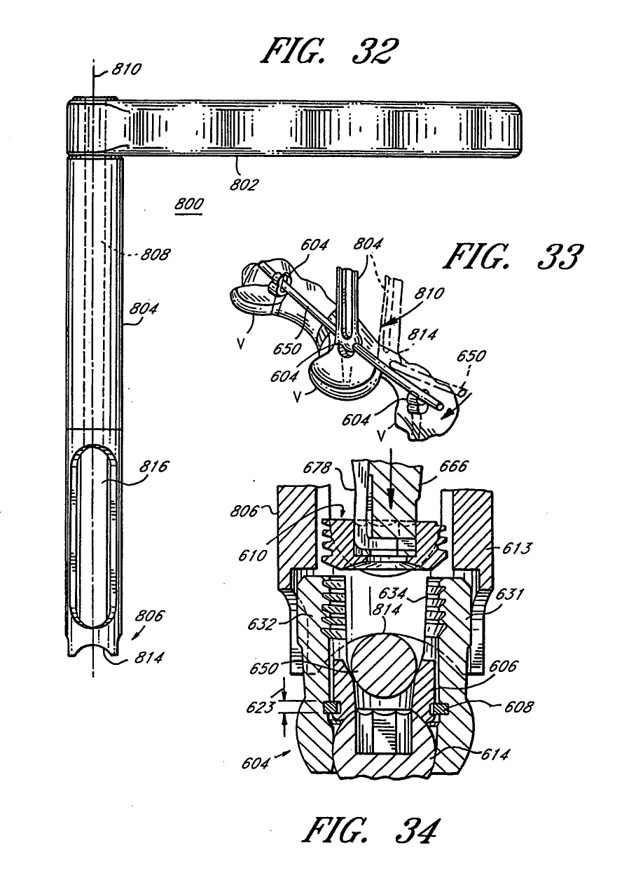

In the illustrated embodiment, this step is performed with a surgical instrument, such as a compressor-distractor instrument 900, illustrated in FIG. 36, which is useful to relatively position bone structures in the cephcaudal direction and to fix their position with respect to one another. Thus, the compressor-distractor instrument 900 has the capability to engage two fasteners 600 and to space them apart while simultaneously tightening one of the fasteners to fix the spacing between the two vertebrae, or other bone structures. Moreover, the compressor-distractor instrument 900 may also be used to move two fasteners 600, and the vertebrae attached thereto into closer approximation and fix the spacing therebetween.

The distal tool portion 902 of one embodiment of the compressor-distractor instrument 900 is illustrated in FIG. 36. The distal tool portion 902 includes a driver portion 904 and a spacing member 906. The driver portion 904 has a distal end portion 908 with a plurality of wrenching flats configured to engage the recess 611 in the proximal face of the cap screw 610, and to apply torque to the cap screw. The driver portion 904 is rotatable about the longitudinal axis (indicated by arrow M) to rotate the cap screw 610 relative to the fastener 600. Accordingly, the driver portion 904 can be rotated to loosen the cap screw 610 on the fastener 600 and permit movement of the elongated member 650 connected with the vertebra relative to the fastener 600 connected with the vertebra. The cap screw 610 can also be rotated in order to tighten the cap screw 610 and clamp the elongated member 650 to the fastener 600.

The distal tool portion 902 may also include a spacing member, such as spacing member 906, which engages an adjacent fastener 600b while driver member 904 is engaged with the housing 604a to move the fastener 600b with respect to the fastener 600a. In the exemplary embodiment, spacing member 906 comprises a jaw portion that is pivotably mounted to move between a first position adjacent the driver portion and a second position spaced from the driver portion, as shown in FIG. 36. The distal tip 910 of the spacing member 906 is movable relative to the driver portion 904 in a direction extending transverse to the longitudinal axis. (Further details and features related to compressor-distractor apparatuses are described in U.S. application Ser. No. 10/178,875, filed Jun. 24, 2002, entitled “Surgical Instrument for Moving Vertebrae,” published as U.S. Patent Application Publication No. 2003/0236529A1 on Dec. 25, 2003, which is incorporated by reference in its entirety herein. Additionally, further details related to instrumentation for moving a vertebra are described in U.S. Pat. No. 6,648,888, issued Nov. 18, 2003; PCT Application No. PCT/US02/28106, filed Sep. 5, 2002, titled SURGICAL INSTRUMENT FOR MOVING VERTEBRAE; PCT Application No. PCT/US03/27879, filed Sep. 5, 2003, titled SURGICAL INSTRUMENT FOR MOVING A VERTEBRAE; and PCT Application No. PCT/US03/04361, filed Feb. 13, 2003, titled APPARATUS FOR CONNECTING A LONGITUDINAL MEMBER TO A BONE PORTION, which are hereby incorporated by reference in their entireties herein.)

As illustrated in FIG. 36, the spacer member 906 can be opened with respect to the driver portion 904 to space the vertebrae farther apart (as indicated by arrow N). The distal portion 910 of the spacer member 906 engages the housing 604b of fastener 600b and moves fastener 600b further apart from fastener 600a to distract the vertebrae. Where the vertebrae are to be moved closer together, e.g. compressed, the spacer member 906 is closed with respect to the driver portion 904 (arrow P), as illustrated in FIG. 37. The distal portion 910 of the spacer member 906 engages the housing 604b of the fastener 600b and moves the fastener 600b towards the fastener 600a. When the spacing of the vertebrae is acceptable to the physician, the cap screw 610a is tightened by the driver member 904, thereby fixing the relationship of the housing 604a with respect to the elongated member 650, and thereby fixing the position of the vertebrae, or other bone structures, with respect to one another. In one application, once the elongated member 650 is fixed with respect to the fasteners 600, the fixation portion of the procedure is substantially complete.

2. Fusion Systems and Devices

Although fixation may provide sufficient stabilization, in some cases it is also desirable to provide additional stabilization. For example, where one or more discs has degraded to the point that it needs to be replaced, it may be desirable to position an implant, e.g., a fusion device, a prosthetic disc, a disc nucleus, etc., in the intervertebral space formerly occupied by the disc.

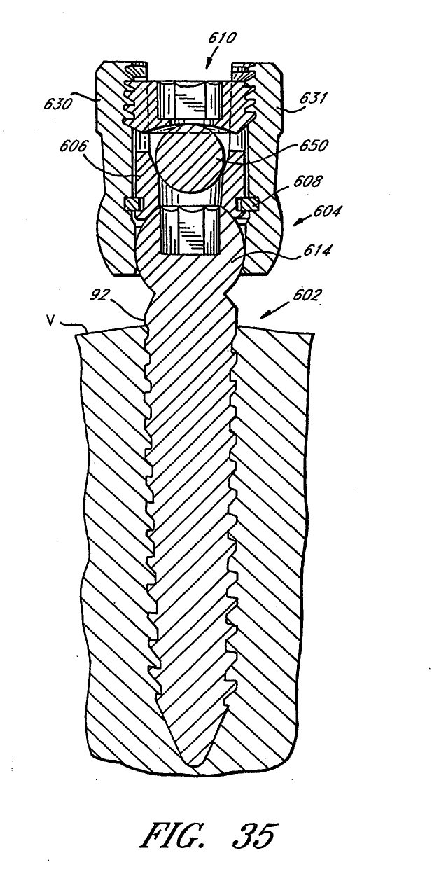

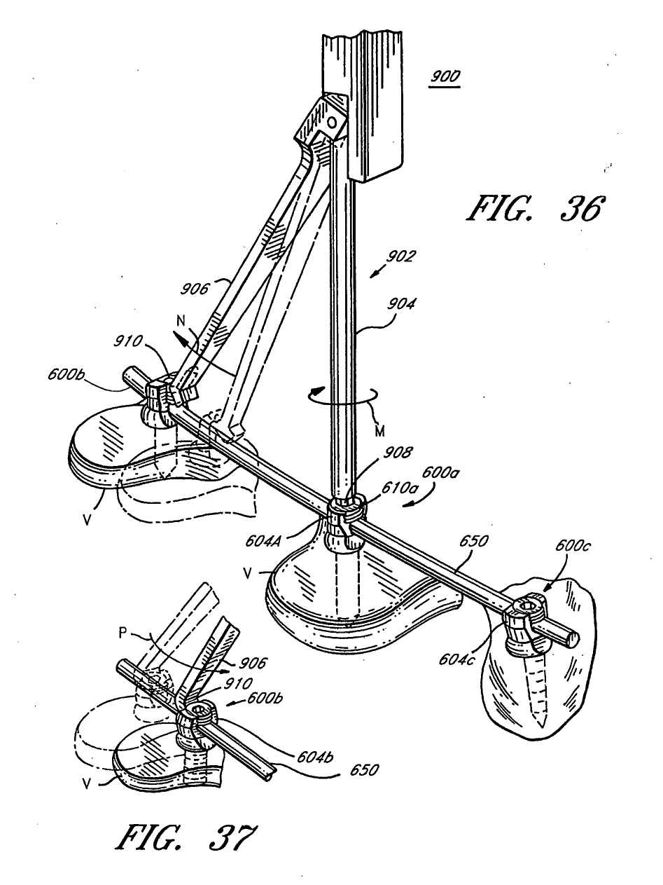

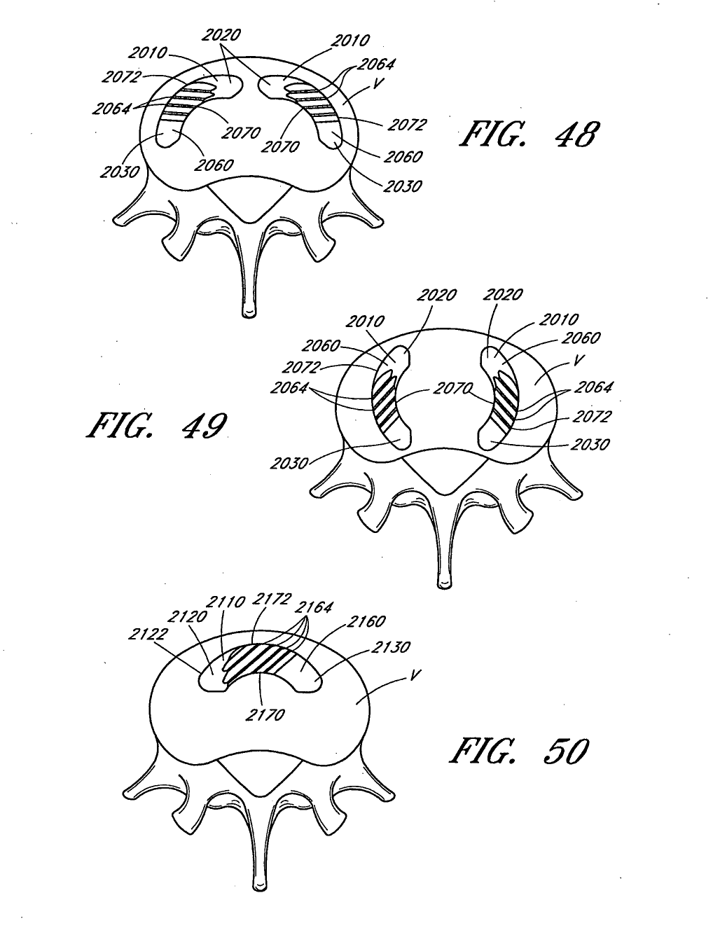

In one application, a fusion device is inserted between adjacent vertebrae V. Portions of the fusion procedure can be performed before, during, or after portions of the fixation procedure. FIGS. 38-42 illustrate one embodiment of a fusion device, referred to herein as a spinal implant 2010, that is inserted between adjacent vertebrae. The spinal implant 2010 preferably is placed between adjacent vertebrae to provide sufficient support to allow fusion of the adjacent vertebrae, as shown in FIGS. 48-49. The spinal implants 2010 are preferably made from an allograft material, though other materials could also be used, including autograft, xenograft, or some non-biologic biocompatible material, such as titanium or stainless steel. Also, where non-biologic materials are used, the implant 2010 may be configured as a cage or other suitable configuration.

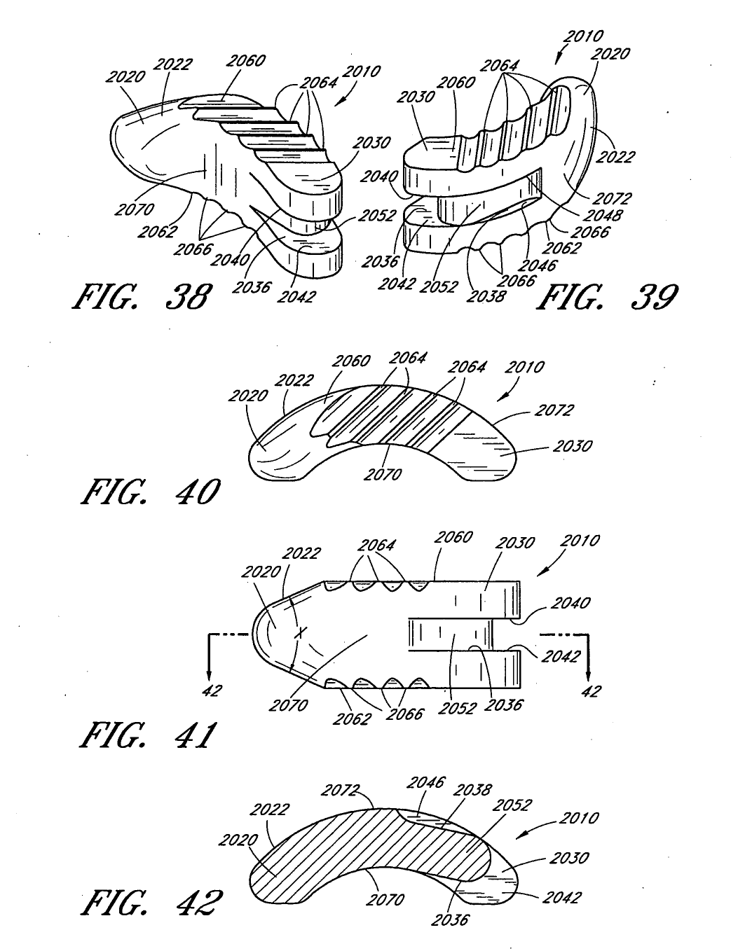

The spinal implant 2010 (FIGS. 38-42) has a first end 2020 for insertion between adjacent vertebrae V. The first end 2020 has a tapered surface 2022 to facilitate insertion of the implant between adjacent vertebrae V. The surface 2022 defines an angle X of approximately 45° as shown in FIG. 41.

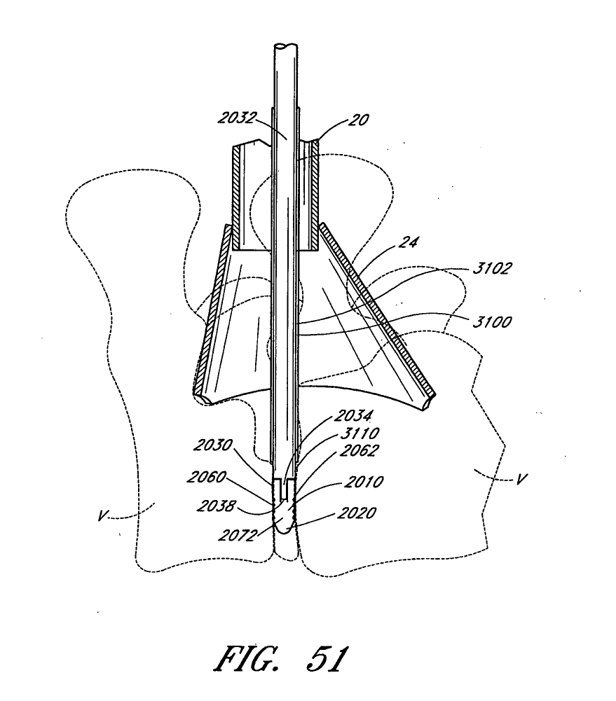

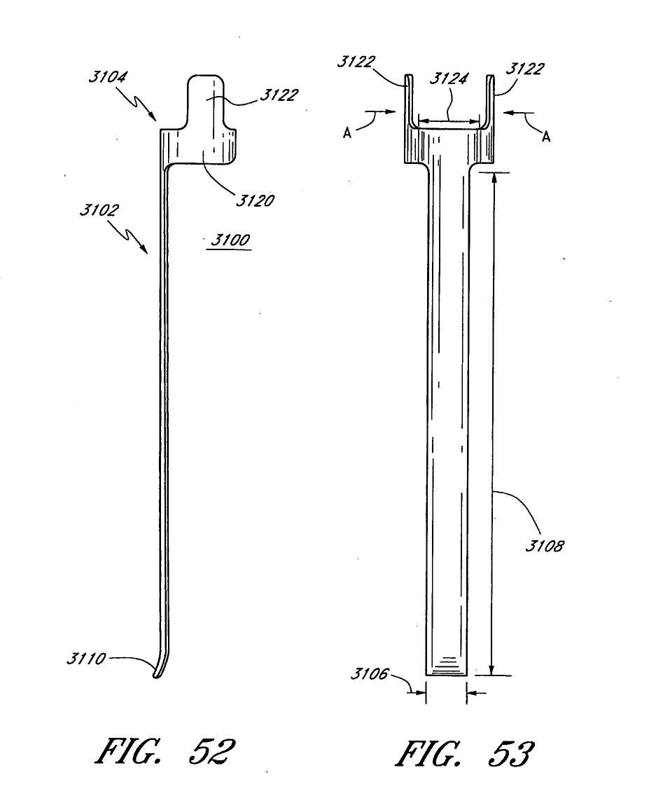

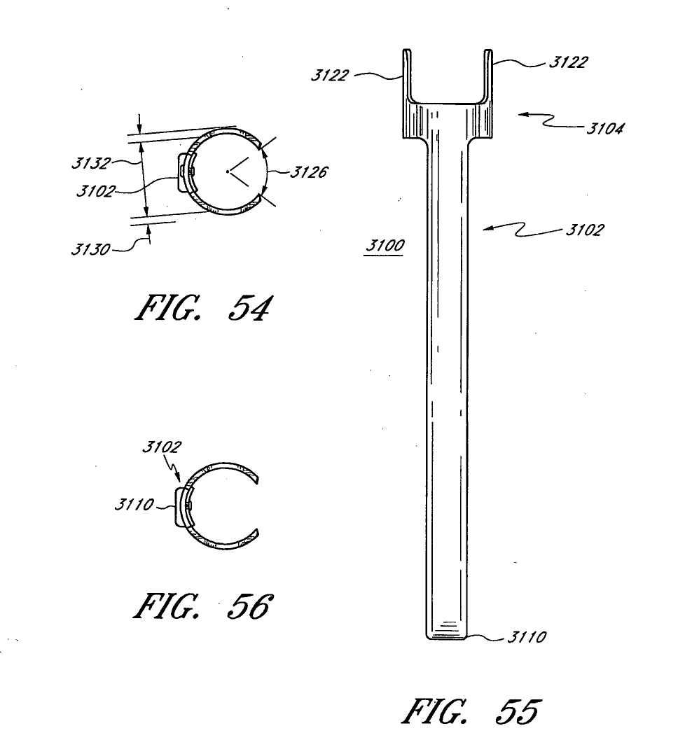

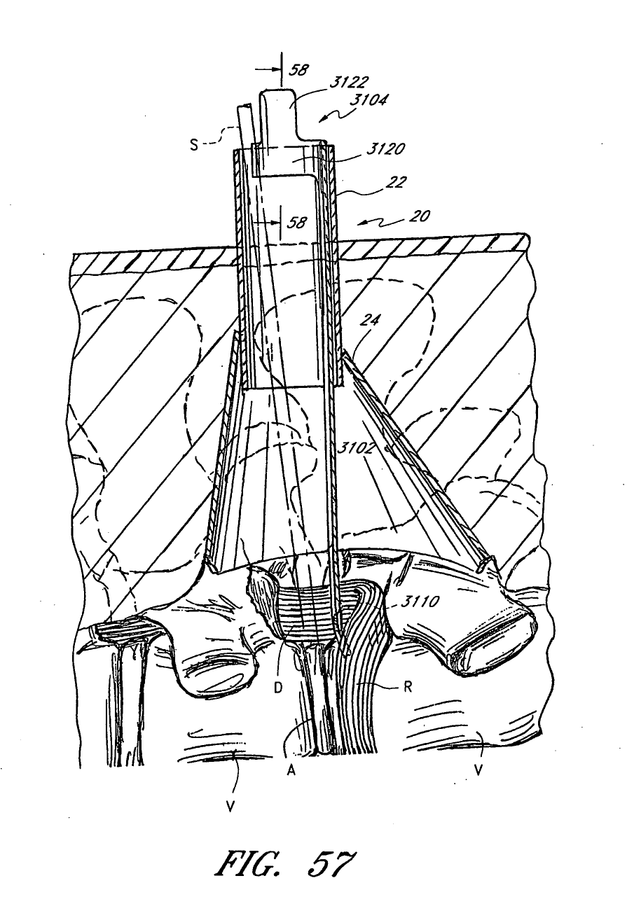

The spinal implant 2010 (FIGS. 38-39) has a second end 2030 that is engageable with a tool 2032 (FIG. 51) for inserting the implant between the adjacent vertebrae V. The tool 2032 has a pair of projections 2034, one of which is shown in FIG. 51, that extend into recesses 2036 and 2038 in the end 2030 of the implant 2010. The recesses 2036 and 2038 (FIGS. 38-39) extend from the second end 2030 toward the first end 2020. The recess 2036 (FIG. 41) is defined by an upper surface 2040 and a lower surface 2042 extending generally parallel to the upper surface 2040. The recess 2038 (FIG. 39) has a lower surface 2046 and an upper surface 2048. The upper surface 2048 extends generally parallel to the lower surface 2046.

The recesses 2036 and 2038 define a gripping portion 2052. The projections 2034 on the tool 2032 extend into the recesses 2036 and 2038 and grip the gripping portion 2052. The projections 2034 engage the upper and lower surfaces 2040 and 2042 of the recess 2036 and the upper and lower surfaces 2046 and 2048 of the recess 2038. Accordingly, the tool 2032 can grip the implant 2010 for inserting the implant between the adjacent vertebrae V.

As viewed in FIGS. 38-41, the implant 2010 has an upper surface 2060 for engaging the upper vertebra V. The implant 2010 has a lower surface 2062, as viewed in FIGS. 38-41, for engaging the lower vertebra V. The upper and lower surfaces 2060 and 2062 extend from the first end 2020 to the second end 2030 of the implant 2010 and parallel to the upper and lower surfaces 2040, 2042, 2046, and 2048 of the recesses 2036 and 2038. The upper surface 2060 has teeth 2064 for engaging the upper vertebra V. The lower surface 2062 has teeth 2066 for engaging the lower vertebra V. Although FIGS. 38-39 show four teeth 2064 and four teeth 2066, it is contemplated that any number of teeth could be used.

A first side surface 2070 and a second side surface 2072 extend between the upper and lower surfaces 2060 and 2062. The first side surface 2070 extends along a first arc from the first end 2022 of the implant 2010 to the second end 2030. The second side surface 2072 extends along a second arc from the first end 2022 to the second end 2030. The first and second side surfaces 2070 and 2072 are concentric and define portions of concentric circles. The teeth 2064 and 2066 extend parallel to each other and extend between the side surfaces 2070 and 2072 and along secant lines of the concentric circles defined by the side surfaces.

The implant 2010 preferably is formed by harvesting allograft material from a femur, as known in the art. The femur is axially cut to form cylindrical pieces of allograft material. The cylindrical pieces are then cut in half to form semi-cylindrical pieces of allograft material. The semi-cylindrical pieces of allograft material are machined into the spinal implants 2010.

A pair of spinal implants 2010 may be placed bilaterally between the adjacent vertebrae V. The access device 20 is positioned in the patient's body adjacent the vertebrae V. The skirt portion 24 of the access device 20 preferably is in a radially expanded condition to provide a working space adjacent the vertebrae V as described above. Disc material between the vertebrae V can be removed using instruments such as kerrisons, rongeurs, or curettes. A microdebrider may also be utilized to remove the disc material. An osteotome, curettes, and scrapers can be used to prepare end plates of the vertebrae V for fusion. Preferably, an annulus of the disc is left between the vertebrae V.

Distracters can be used to sequentially distract the disc space until the desired distance between the vertebrae V is achieved. The fusion device or implant 2010 is placed between the vertebrae V using the tool 2032. The first end 2020 of the implant 2010 is inserted first between the vertebrae V. The implant 2010 is pushed between the vertebrae V until the end 2030 of the implant is between the vertebrae. A second spinal implant 2010 is inserted on the ipsilateral side using the same procedure.

A shield apparatus 3100 with an elongated portion 3102 may be used to facilitate insertion of the implants 2010 between the vertebrae V. A distal portion 3110 of the apparatus 3100 may be placed in an annulotomy. The implant 2010 is inserted with the side surface 2170 facing the elongated portion 3102 so that the apparatus 3100 can act as a “shoe horn” to facilitate or guide insertion of the implants 2010 between the vertebrae.

The implants 2010 may be inserted between the vertebrae V with the first ends 2020 located adjacent each other and the second ends 2030 spaced apart from each other, as shown in FIG. 48. The implants 2010 may also be inserted between the vertebrae V with the first ends 2020 of the implants 2010 spaced apart approximately the same distance that the second ends 2030 are spaced apart. It is contemplated that the implants 2010 may be inserted in any desired position between the vertebrae V. It is also contemplated that in some embodiments only one implant 2010 may be inserted between the vertebrae V. Furthermore, it is contemplated that the implants 2010 may be inserted between vertebrae using an open procedure.

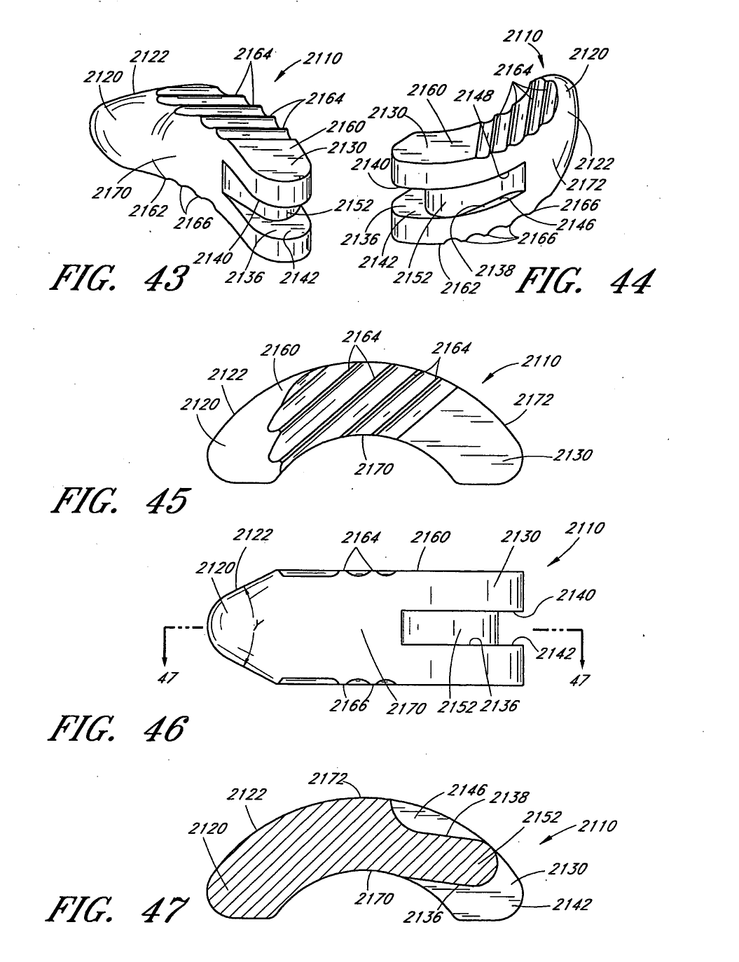

Another embodiment of a fusion device or spinal implant 2110 is illustrated in FIGS. 43-47. The spinal implant 2110 is substantially similar to the embodiment disclosed in FIGS. 38-42. The implant 2110 is placed between the adjacent vertebrae V to provide sufficient support to allow fusion of the adjacent vertebrae, as shown in FIG. 50. The spinal implant 2110 is preferably made from an allograft material, though the materials described above in connection with the spinal implant 2010 may also be used. Also, as with the implant 2010, the implant 2110 may be formed as a cage or other suitable configuration.

The spinal implant 2110 (FIGS. 43-47) has a first end 2120 for insertion between the adjacent vertebrae V. The first end 2120 has a tapered surface 2122 to facilitate insertion of the implant between the adjacent vertebrae V. The surface 2122 defines an angle Y of approximately 45° as shown in FIG. 65.

The spinal implant 2110 (FIGS. 43-44) has a second end 2130 that is engageable with the projections 2034 on the tool 2032 for inserting the implant between the adjacent vertebrae V. The projections 2034 extend into recesses 2136 and 2138 in the end 2130 of the implant 2110. The recesses 2136 and 2138 extend from the second end 2130 toward the first end 2120. The recess 2136 (FIGS. 43 and 46) is defined by an upper surface 2140 and a lower surface 2142 extending generally parallel to the upper surface 2140. The recess 2138 (FIG. 44) has a lower surface 2146 and an upper surface 2148 extending generally parallel to the lower surface 2146.

The recesses 2136 and 2138 define a gripping portion 2152. The projections 2034 on the tool 2032 extend into the recesses 2136 and 2138 and grip the gripping portion 2152. The projections 2034 engage the upper and lower surfaces 2140 and 2142 of the recess 2136 and the upper and lower surfaces 2146 and 2148 of the recess 2138. Accordingly, the tool 2032 can grip the implant 2110 for inserting the implant between the adjacent vertebrae V.

As viewed in FIGS. 43-46, the implant 2110 has an upper surface 2160 for engaging the upper vertebra V. The implant 2110 has a lower surface 2162, as viewed in FIGS. 43-46, for engaging the lower vertebra V. The upper and lower surfaces 2160 and 2162 extend from the first end 2120 to the second end 2130 of the implant 2110 and parallel to the upper and lower surfaces 2140, 2142, 2146, and 2148 of the recesses 2136 and 2138. The upper surface 2160 has teeth 2164 for engaging the upper vertebra V. The lower surface 2162 has teeth 2166 for engaging the lower vertebra V. Although FIG. 44 shows four teeth 2164 and four teeth 2166, it is contemplated that any number of teeth could be used.

A first side surface 2170 and a second side surface 2172 extend between the upper and lower surfaces 2160 and 2162. The first side surface 2170 extends along a first arc from the first end 2122 of the implant 2110 to the second end 2130. The second side surface 2172 extends along a second arc from the first end 2120 to the second end 2130. The first and second side surfaces 2170 and 2172 are concentric and define portions of concentric circles. The teeth 2164 and 2166 extend parallel to each other and between the side surfaces 2170 and 2172 along secant lines of the concentric circles defined by the side surfaces.

The implant 2110 preferably is formed by harvesting allograft material from a femur, as is known in the art. The femur is axially cut to form cylindrical pieces of allograft material. The cylindrical pieces are then cut in half to form semi-cylindrical pieces of allograft material. The semi-cylindrical pieces of allograft material are machined into the spinal implants 2110.

A spinal implant 2110 is placed unilaterally between the adjacent vertebrae V. The access device 20 is positioned in the patient's body adjacent the vertebrae V. The skirt portion 24 of the access device 20 preferably is in a radially expanded condition to provide a working space adjacent the vertebrae V as described above. Disc material between the vertebrae V can be removed using instruments such as kerrisons, rongeurs, or curettes. A microdebrider may also be utilized to remove the disc material. An osteotome, curettes, and scrapers can be used to prepare end plates of the vertebrae V for fusion. Preferably, an annulus of the disc is left between the vertebrae V.

Distracters are used to sequentially distract the disc space until the desired distance between the vertebrae V is achieved. The implant 2110 is placed between the vertebrae V using the tool 2032. It is contemplated that the apparatus 3100 could be used also. The first end 2120 of the implant 2110 is inserted first between the vertebrae V. The implant 2110 is pushed between the vertebrae V until the end 2130 of the implant is between the vertebrae. It is contemplated that the implant 2110 may be inserted in any desired position between the vertebrae V. It is also contemplated that in some embodiments more than one implant 2110 may be inserted between the vertebrae.

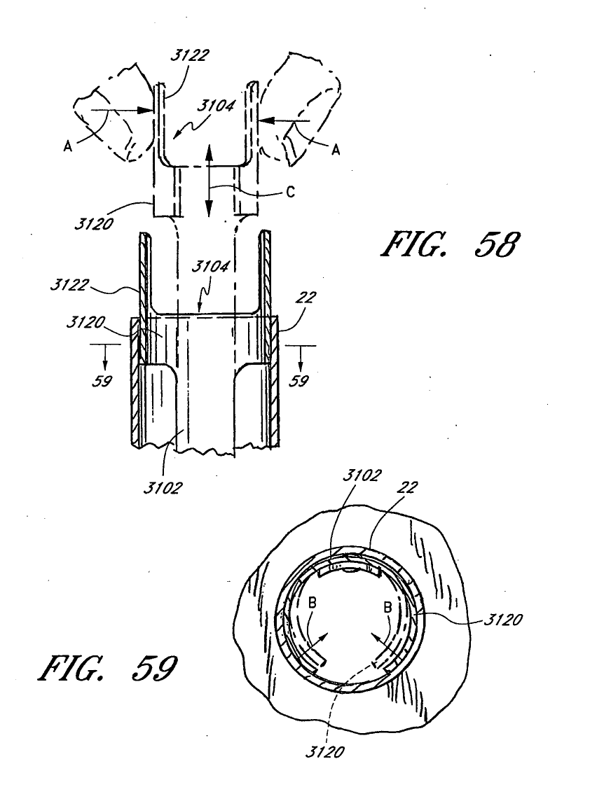

The apparatus or shield 3100 for use in placing the fusion devices or spinal implants between the vertebrae is illustrated in FIGS. 52-56. The apparatus 3100 preferably includes an elongated body portion 3102, which protects the nerve root or dura, and a mounting portion 3104, which allows for the surgeon to releasably mount the apparatus 3100 to the access device 20. Consequently, the surgeon is able to perform the surgical procedures without requiring the surgeon or an assistant to continue to support the apparatus 3100 throughout the procedure, and without reducing the field of view.

The apparatus 3100 may be manufactured from a biocompatible material such as, for example, stainless steel. In the illustrated embodiment, apparatus 3100 is manufactured from stainless steel having a thickness of about 0.02 inches to about 0.036 inches. The elongated body portion 3102 has dimensions that correspond to the depth in the body in which the procedure is being performed, and to the size of the body structure that is to be shielded by elongated body portion 3102. In the exemplary embodiment, the elongated body portion 3102 has a width 3106 of about 0.346 inches and a length of about 5.06 inches (FIG. 53), although other dimensions would be appropriate for spinal surgical procedures performed at different locations, or for surgical procedures involving different body structures. The distal tip portion 3110 of the apparatus 3100 may have a slightly curved “bell mouth” configuration which allows for atraumatic contact with a body structure, such as a nerve. It is contemplated that the elongated body portion may have any desired shape.

The mounting portion 3104 preferably allows the apparatus 3100 to be secured to a support structure in any number of ways. In the exemplary embodiment, mounting portion 3104 may include a ring portion. With reference to FIGS. 52-56, ring portion 3120 has a substantially ring-shaped configuration with an opening 3124, which defines an angle 3126 of about 90 degrees of the total circumference of the ring portion 3120. As will be described in greater detail below, the angle 3126 is a nominal value, because the ring portion 3104 is resilient, which permits the opening 3124 to change size during the mounting process.

In the illustrated embodiment, the mounting portion 3104 has a substantially cylindrical configuration in order to be mounted within the interior lumen of the access device 20, as will be described below. The ring portion 3104 has an exterior dimension 3130 of about 0.79 inches, and an interior dimension 3132 of about 0.76 inches. It is understood that the dimensions of the ring portion 3104 can be different, such as, for example, where the access device 20 has a different interior dimension. Moreover, the cylindrical shape of the ring portion 3104 can change, such as, for example, where the apparatus 3100 is used with a support member having a differently shaped internal lumen.

Finger grip portions 3122 preferably extend from the mounting portion 3104 and allow the surgeon to apply an inwardly directed force (as indicated by arrows A) to the ring portion 3120. The resilient characteristics of the ring portion 3120 allow the material to deflect thereby reducing the exterior dimension 3130 and reducing the spacing 3124. Releasing the finger grip portions 3122 allows the ring portion to move towards its undeflected condition, thereby engaging the interior wall of the access device 20.

The elongated body portion 3102 and the mounting portion 3104 may be manufactured from a single component, such as a sheet of stainless steel, and the mounting portion 3104 may be subsequently formed into a substantially cylindrical shape. In another embodiment, the mounting portion 3104 may be manufactured as a separate component and coupled to the elongated body portion, by techniques such as, for example, welding and/or securement by fasteners, such as rivets.

The access device 20 serves as a stable mounting structure for apparatus 3100. In particular, mounting portion 3104 is releasably mounted to the interior wall of proximal wall portion 22 of access device 20. Elongated body portion 3102 extends distally into the operative site to protect the desired body structure, such as the nerve, as will be described below.

To install the apparatus 3100 within the interior passage of the proximal wall portion 22, the surgeon may apply an inwardly directed force on the ring portion 3120, thereby causing the ring portion to resiliently deform, as illustrated by dashed line and arrows B in FIG. 59. The surgeon subsequently inserts the apparatus 3100 into the interior lumen of the proximal wall portion 22 (as indicated by arrow C) to the position of ring portion 3104 illustrated in solid line in FIG. 58. When the surgeon releases the finger grip portions 3122, the ring portion 3120 resiliently moves towards its undeflected configuration, thereby engaging the interior lumen of the proximal wall portion 22. Advantages of some embodiments include that the mounting portion 3104 is easily removed and/or moved with respect to the access device 20 without disturbing the position of the access device 20 or any other instrumentation.

As illustrated in FIG. 57, the configuration of the mounting portion 3104 and the elongated body portion 3102 allow the elongated body portion to occupy a small space along the periphery of the proximal wall portion 3122. This allows the apparatus to protect the desired body structure without blocking access for the insertion of other surgical instrumentation, and without blocking visibility by the surgeon during the procedure.

The mounting portion 3104 is one exemplary configuration for mounting the apparatus 3100 to the support structure. It is contemplated that the apparatus 3100 may be mounted within the access device 20 in any suitable manner.

When in position, the distal end portion 3110 covers the exiting nerve root R, while exposing the disc annulus A (See FIG. 57). As discussed above, the debridement and decortication of tissue covering the vertebrae, as well as a facetectomy and/or laminectomy if indicated, are preferably performed prior to the insertion of apparatus 3100 into the surgical space. Accordingly, in some embodiments, there is no need to displace or retract tissue, and apparatus 3100 merely covers the nerve root and does not substantially displace the nerve root or any other body tissue. It is understood that the term “cover” as used herein refers to apparatus 3100 being adjacent to the body structure, or in contact with the body structure without applying significant tension or displacement force to the body structure.

Additional surgical instrumentation S may be inserted into the access device to perform procedures on the surrounding tissue. For example, an annulotomy may be performed using a long handled knife and kerrisons. A discectomy may be completed by using curettes and rongeurs. Removal of osteophytes which may have accumulated between the vertebrae may be performed using osteotomes and chisels.

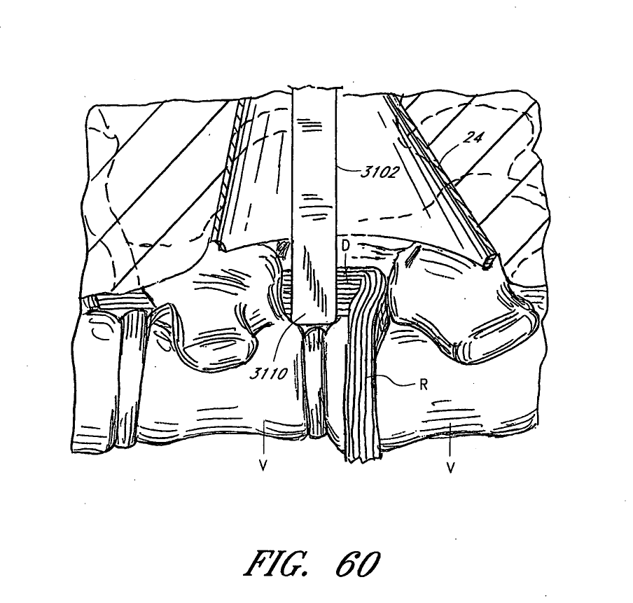

As illustrated in FIG. 60, the elongated body portion 3102 preferably is rotated to protect the spinal cord, or dura D, during the above procedures. The surgeon may change the position of the apparatus 3100 by approximating the finger grips 3122 to release the ring portion from engagement with the inner wall of the proximal wall portion 20, and then re-position the apparatus 3100 without disturbing the access device 20 (as shown in FIG. 58).

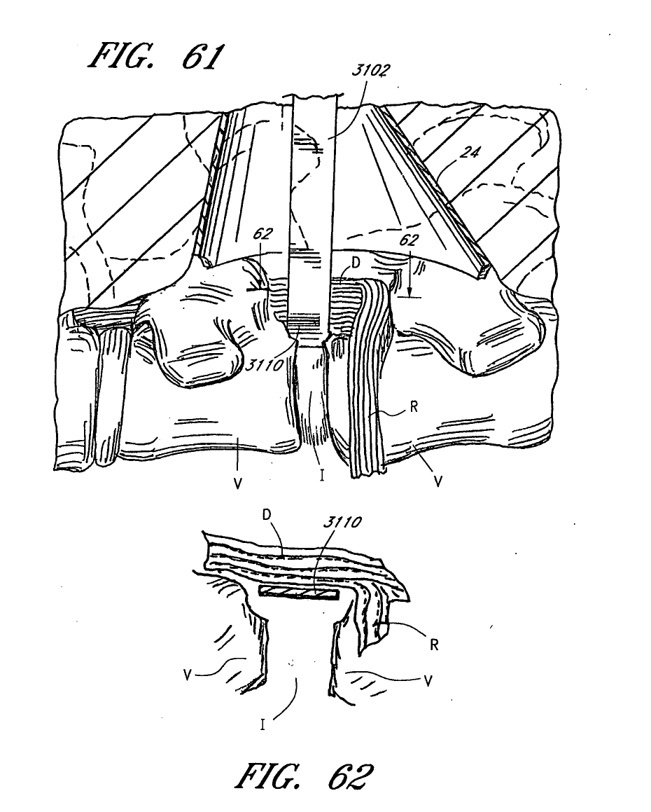

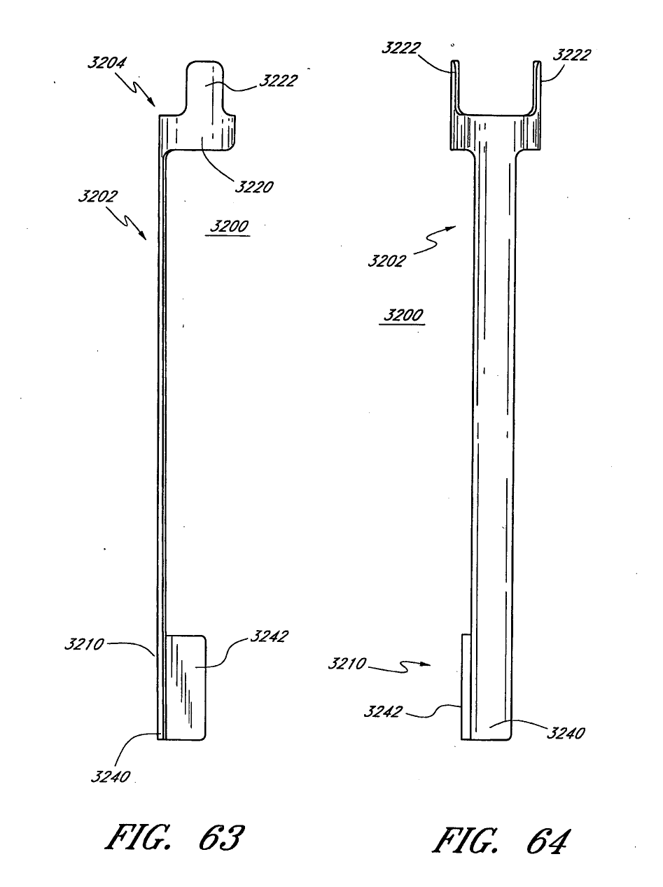

During certain surgical procedures, it may be useful to introduce crushed bone fragments or the fusion devices 2010 or 2110 to promote bone fusion. As illustrated in FIGS. 61-62, apparatus 3100 is useful to direct the implants into the space I between adjacent vertebrae V. As shown in the figures, the distal portion 3110 of the elongated body portion 3102 is partially inserted into the space I. The distal end portion 3110, is positioned between adjacent vertebrae V, and creates a partially enclosed space for receiving the implants or other material therein.

Another embodiment of the apparatus or shield is illustrated in FIGS. 63-64, and designated apparatus 3200. Apparatus 3200 is substantially identical to apparatus 3100, described above, with the following differences noted herein. In particular, distal end portion 3210 includes a pair of surfaces 3240 and 3242. Surface 3240 is an extension of elongated shield portion 3202, and surface 3242 extends at an angle with respect to surface 3240. In the exemplary embodiment, surfaces 3240 and 3242 defined an angle of about 90 degrees between them. Alternatively another angle between surfaces 3240 and 3242 may be defined as indicated by the body structures to be protected.

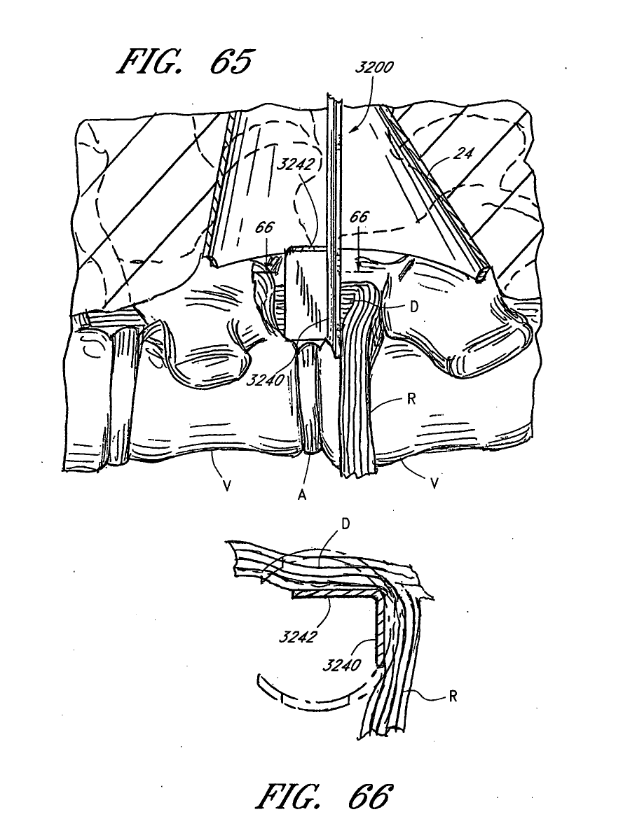

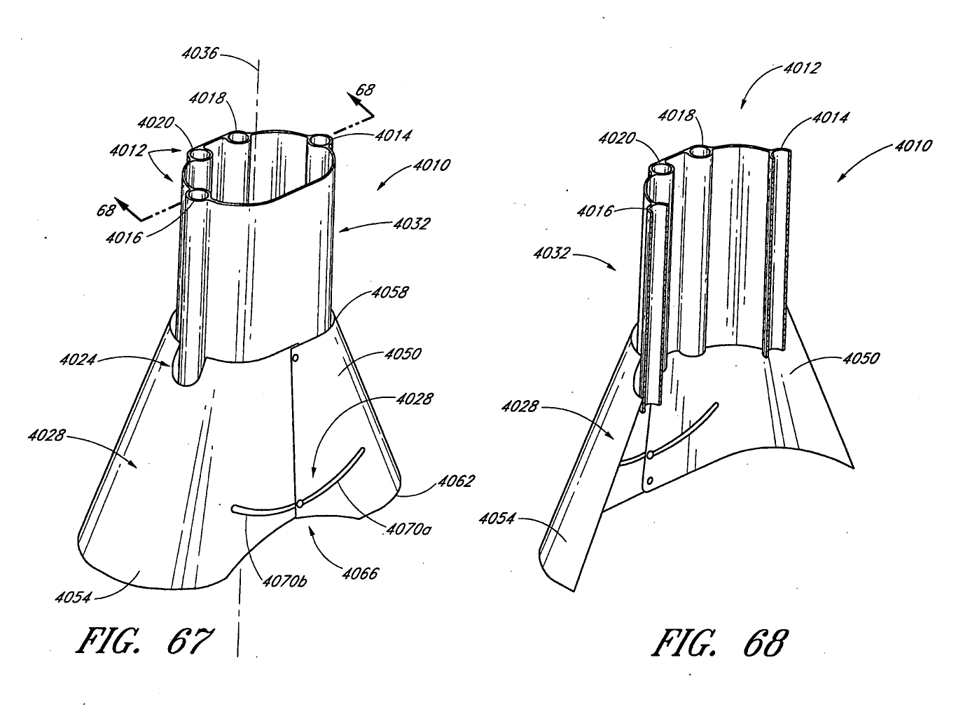

Distal end portion 3210 allows the apparatus to provide simultaneous shielding of both the dura D and the nerve root R. In FIGS. 65-66, surface 3242 shields the dura D, and surface 3240 shields the nerve root R. It is understood that surfaces 3240 and 3242 may be interchanged with respect to which tissue they protect during the surgical procedure.

According to the exemplary embodiment, once the fusion and fixation portions of the procedure have been performed, the procedure is substantially complete. The surgical instrumentation, such as the endoscope 500 can be withdrawn from the surgical site. The access device 20 is also withdrawn from the site. The muscle and fascia typically close as the access device 20 is withdrawn through the dilated tissues in the reduced profile configuration. The fascia and skin incisions are closed in the typical manner, with sutures, etc. The procedure described above may be repeated for the other lateral side of the same vertebrae, if indicated.

### II. Surgical Procedures that May be Performed with the Systems Described Herein

As discussed above, the systems disclosed herein provide access to a surgical location at or near the spine of a patient to enable procedures on the spine. These procedures can be applied to one or more vertebral levels, as discussed above. Additional procedures and combinations of procedures that may be performed using the systems described herein are discussed below. In various forms, these procedures involve an anterior lumbar interbody fusion, a minimally invasive lumbar interbody fusion, and other procedures particularly enabled by the access devices and systems described above.

A. Procedures Involving Anterior Lumbar Interbody Fusion

The access devices and systems described herein are amenable to a variety of procedures that may be combined with an anterior lumbar interbody fusion (referred to herein as an “ALIF”).

In one embodiment of a first method, three adjacent vertebrae, such as the L4, the L5, and the S1 vertebrae of the spine, are treated by first performing an ALIF procedure. Such a procedure may be performed in a convention manner. The ALIF involves exposing a portion of the spine, in particular the vertebrae and discs located in the interbody spaces, i.e., the spaces between adjacent vertebrae. Any suitable technique for exposing the interbody spaces may be employed, e.g., an open, mini-open, or minimally invasive procedure. In one embodiment, the interbody spaces between the L4, L5, and S1 vertebrae are exposed to the surgeon. Once exposed, the surgeon may prepare the interbody space, if needed, in any suitable manner. For example, some or all of the disc may be removed from the interbody space and the height of the interbody space may be increased or decreased. The interbody space between the L4 and the L5 vertebrae may be exposed separately from the interbody space between the L5 and S1 vertebrae or they may be generally simultaneously exposed and prepared.

After the interbody space has been exposed and prepared, a suitable fusion procedure may be performed. For example, in one example fusion procedure, one or more fusion devices may be placed in the interbody space. Any suitable fusion device may be used, e.g., a fusion cage, a femoral ring, or another suitable implant. Various embodiments of implants and techniques and tools for the insertion of implants are described in U.S. application Ser. No. 10/280,489, filed Oct. 25, 2002, which has been published as Publication No. 2003/0073998 on Apr. 17, 2003, which is hereby incorporated by reference herein in its entirety. In one variation, one or more fusion cages may be placed in an interbody space, e.g., between the L4 and L5 vertebrae, between the L5 and S1 vertebrae, or between the L4 and L5 vertebrae and between the L5 and S1 vertebrae. In another variation, one or more femoral rings may be substituted for one or more of the fusion cages and placed between the L4 and L5 vertebrae and/or between the L5 and S1 vertebrae. In another variation, one or more fusion devices are combined with a bone growth substance, e.g., bone chips, to enhance bone growth in the interbody space(s).

After anterior placement of the fusion device, an access device is inserted into the patient to provide access to a spinal location, as described above. A variety of anatomical approaches may be used to provide access to a spinal location using the access device 20. The access device preferably is inserted generally posteriorly. As used herein the phrase “generally posteriorly” is used in its ordinary sense and is a broad term that refers to a variety of surgical approaches to the spine that may be provided from the posterior side, i.e., the back, of the patient, and includes, but is not limited to, posterior, postero-lateral, retroperitoneal, and transforaminal approaches. Any of the access devices described or incorporated herein, such as the access device 20, could be used.

The distal end of the access device may be placed at the desired surgical location, e.g., adjacent the spine of the patient with a central region of the access device over a first vertebrae. In one procedure, the distal end of the access device is inserted until it contacts at least a portion of at least one of the vertebrae being treated or at least a portion of the spine. In another procedure, the distal end of the access device is inserted until it contacts a portion of the spine and then is withdrawn a small amount to provide a selected gap between the spine and the access device. In other procedures, the access device may be inserted a selected amount, but not far enough to contact the vertebrae being treated, the portion of the vertebrae being treated, or the spine.

The access device may be configured, as described above, to provide increased access to the surgical location. The access device can have a first configuration for insertion to the surgical location over the first vertebra and a second configuration wherein increased access is provided to the adjacent vertebrae. The first configuration may provide a first cross-sectional area at a distal portion thereof. The second configuration may provide a second cross-sectional area at the distal portion thereof. The second cross-sectional area preferably is enlarged compared to the first cross-sectional area. In some embodiments, the access device may be expanded from the first configuration to the second configuration to provide access to the adjacent vertebrae above and below the first vertebra.

When it is desired to treat the L4, L5, and S1 vertebrae, the access device may be inserted over the L5 vertebrae and then expanded to provide increased access to the L4 and S1 vertebrae. In one embodiment, the access device can be expanded to an oblong shaped configuration wherein the access device provides a first dimension of about 63 mm, and a second dimension perpendicular to the first dimension of about 24 mm. In another embodiment, the access device can be expanded to provide a first dimension of about 63 mm, and a second dimension perpendicular to the first dimension of about 27 mm. These dimensions provide a surgical space that is large enough to provide access to at least three adjacent vertebrae without exposing excessive amounts of adjacent tissue that is not required to be exposed for the procedures being performed. Other dimensions and configurations are possible that would provide the needed access for procedures involving three adjacent vertebrae.

When the access device is in the second configuration, fixation of the three vertebrae may be performed. As discussed above, fixation is a procedure that involves providing a generally rigid connection between at least two vertebrae. Any of the fixation procedures discussed above could be used in this method, as could other fixation procedures. One fixation procedure that could be used is discussed above in connection with FIG. 36 wherein the fasteners 600a, 600b, and 600c are advanced through the access device 20 to three adjacent vertebrae and are attached to the vertebrae. The three fasteners 600a, 600b, and 600c are interconnected by the elongated member 650. The three fasteners 600a, 600b, and 600c and the elongate member 650 comprise a first fixation assembly. A second fixation assembly may be applied to the patient on the opposite side of the spine, i.e., about the same location on the opposite side of the medial line of the spine. Other fixation procedures could be applied, e.g., including two fasteners that coupled to the L4 and the S1 vertebrae and an elongate member interconnecting these vertebrae.

One variation of the first method provides one level of fixation on the anterior side of the patient, e.g., when the fusion device is placed in the interbody space. For example, fixation of the L5 and S1 vertebrae could be provided on the anterior side of the spine, in addition to the other procedures set forth above (e.g., a two level postero-lateral fixation). Also, fixation of the L4 and L5 vertebrae could be provided on the anterior side of the spine, in addition to the other procedures set forth above (e.g., a two level postero-lateral fixation).

In a second method, substantially the same steps as set forth above in connection with the first method would be performed. In addition, after the access device is inserted, a decompression procedure is performed through the access device. A decompression procedure is one where unwanted bone is removed from one or more vertebrae. Unwanted bone can include stenotic bone growth, which can cause impingement on the existing nerve roots or spinal cord. Decompression procedures that may be performed include laminectomy, which is the removal of a portion of a lamina(e), and facetectomy, which is the removal of a portion of one or more facets. In one variation of this method, decompression includes both a facetectomy and a laminectomy. Any suitable tool may be used to perform decompression. One tool that is particularly useful is a kerrison.

In a third method, substantially the same steps as set forth above in connection with the first method would be performed. That is, an ALIF procedure is performed in combination with a fixation procedure. In addition, a fusion procedure may be performed through the access device which may have been placed generally posteriorly, e.g., postero-laterally, tranforaminally or posteriorly, whereby bone growth is promoted between the vertebrae and the fixation assembly, including at least one of the fasteners 600a, 600b, 600c and/or the elongate element 650. This procedure is also referred to herein as an “external fusion” procedure.

One example of an external fusion procedure that may be performed involves placement of a substance through the access device intended to encourage bone growth in and around the fixation assembly. Thus, fusion may be enhanced by placing a bone growth substance adjacent any of the fasteners 600a, 600b, 600c and/or the elongate member 650. The bone growth substance may take any suitable form, e.g., small bone chips taken from the patient (e.g., autograft), from another donor source (e.g., allograft or xenograft), and orthobiologics.

After the bone growth substance is applied to the fixation assembly, the access device is removed. Absent the retracting force provided by the access device, the patient's tissue generally collapses onto the bone growth substance. The tissue will thereby maintain the position of the bone growth substance adjacent to the fixation assembly. The presence of the bone growth substance can cause bone to bridge across from the vertebra(e) to one or more components of the fixation assembly.

In a fourth method, substantially the same steps as set forth above in connection with the second method would be performed. That is, an ALIF procedure is performed anteriorly, and a decompression procedure and a fixation procedure are performed through the access device which may be placed generally posteriorly, e.g., postero-laterally, tranforaminally, or posteriorly. In addition, bone growth substance is placed in and around a fixation assembly through the access device, as discussed above in connection with the third method. The bone growth substance encourages bone to bridge across from the vertebrae to the fixation assembly.

In a fifth method, an ALIF procedure is performed, as discussed above in connection with the second method. After one or more fusion devices is placed in the interbody space, access is provided by way of the access device, as discussed above, from any suitable anatomical approach, e.g., a generally posterior approach. Preferably, a postero-lateral approach is provided. After access has been provided, a bone growth substance, such as those discussed above in connection with the third method, is delivered through the access device. The bone growth substance is placed adjacent an interbody space, e.g., the space between the L4 and the L5 vertebrae and/or between the L5 and the S1 vertebrae. The bone growth substance encourages fusion of the adjacent vertebrae, e.g., L4 to L5 and/or L5 to S1, by stimulating or enhancing the growth of bone between adjacent vertebrae, as discussed above.

In a sixth method, substantially the same steps described in connection with the first method are performed, except that the fixation procedure is optional. In one variation of the sixth method, the fixation procedure is not performed. However, after the access device is inserted, a bone growth substance is placed in and around one or more interbody spaces through the access device. Where the sixth method involves a two level procedure, the bone growth substance can be placed adjacent the interbody space between the L4 and the L5 vertebra and/or between the L5 and the S1 vertebra. Thus, bone growth may occur in the interbody space and adjacent the interbody space between the vertebrae.

The foregoing discussion illustrates that an ALIF procedure can be combined with a variety of procedures that can be performed through an access device disclosed herein. In addition, though not expressly set forth herein, any combination of the procedures discussed above, and any other suitable known procedure, may also be combined and performed through the access devices described herein, as should be understood by one skilled in the art.

B. Spine Procedures Providing Minimally Invasive Lumbar Interbody Fusion

Another category of procedures that may be performed with the access devices and systems described above involves a minimally invasive lumbar interbody fusion (referred to herein as a “MILIF”). MILIF procedures are particularly advantageous because they permit the surgeon to perform a wide variety of therapeutic procedures without requiring fusion by way of an anterior approach, as is required in an ALIF. This provides a first advantage of allowing the surgeon to perform all procedures from the same side of the patient and also possibly from the same approach. Also, the access devices and systems disclosed herein provide the further advantage of enabling two level procedures, and many other related procedures, to be performed by way of a single percutaneous access. These and other advantages are explained more fully below.

In a first MILIF method, a two level postero-lateral fixation of the spine involving three adjacent vertebrae, such as the L4, L5, and S1 vertebrae, is provided. Analogous one level procedures and two level procedures involving any other three vertebrae also may be provided. In addition, the access devices and systems described herein could be used or modified to accommodate other multi-level procedures, such as a three level procedure. The surgeon inserts an access device such as described herein to a surgical location near the spine. As discussed above, the access devices are capable of a wide variety of anatomical approaches. In this procedure, a postero-lateral approach is preferred. Once the access device is inserted to a location adjacent the spine, as discussed above, it may be configured, e.g., expanded, as discussed above, to a configuration wherein sufficient access is provided to the surgical location.

Any suitable fusion process may then be performed. For example, an implant may be advanced through the access device into the interbody space in order to maintain disc height and allow bone growth therein, e.g., as in a fusion procedure. In order to ease insertion of the implant, it may be beneficial to prepare the interbody space. Interbody space preparation may involve removal of tissue or adjusting the height of the interbody space through the access device, such as in a distraction procedure. Once the interbody space is prepared, a suitable implant may be advanced through the access device into the interbody space, taking care to protect surrounding tissues. Various embodiments of implants and techniques and tools for their insertion are described in U.S. application Ser. No. 10/280,489, incorporated by reference hereinabove. In general, the implant preferably is an allograft strut that is configured to maintain disc height and allow bone growth in the interbody space.

In addition to providing a suitable fusion, the first method provides fixation of the vertebrae. The fixation procedure may take any suitable form, e.g., any of the fixation procedures similar to those disclosed above. In particular, when the access device is in the expanded or enlarged configuration, fixation of the three adjacent vertebrae may be performed. One fixation procedure that could be used is discussed above in connection with FIG. 36 wherein the fasteners 600a, 600b, and 600c are advanced through the access device 20 to three adjacent vertebrae and are attached to the vertebrae. The three fasteners 600a, 600b, and 600c are interconnected by way of the elongated member 650. As discussed above, a second fixation assembly may be applied to the patient on the opposite side of the spine, e.g., about the same location on the opposite side of the medial line of the spine.

In a second MILIF method, substantially the same procedures set forth above in connection with the first MILIF method are performed. In addition, a suitable decompression procedure may be performed, as needed. As discussed above, decompression involves removal of unwanted bone by way of a suitable decompression technique that may be performed through the access device. In one embodiment, decompression is performed through the access device after the access device has been expanded. As discussed above, suitable decompression techniques include a laminectomy, a facetectomy, or any other similar procedure. Decompression for the L4, the L5, and/or the S1 vertebrae may be needed and can be performed through the access devices described herein without requiring the access device to be moved from one position to another.

In a third MILIF method, substantially the same procedures set forth above in connection with the first MILIF method are performed. In addition, a further fusion procedure, e.g., a fusion procedure external to the interbody space, is provided. The external fusion procedure is performed adjacent to the interbody space wherein bone growth may be promoted in the proximity of the fixation assembly, e.g., above the postero-lateral boney elements of the spine, such as the facet joints and the transverse processes. In one embodiment, when the fixation assembly comprising the fasteners 600a, 600b, 600c and/or the elongate element 650 has been applied to three adjacent vertebrae, a substance is applied through the access device to one or more components of the fixation assembly to maintain or enhance the formation and/or growth of bone in the proximity of the fixation assembly. For example, a bone growth substance may be placed adjacent any of the fasteners 600a, 600b, 600c and/or the elongate member 650. Bone growth substance may take any suitable form, e.g., small bone chips taken from the patient (e.g., autograft), from another donor source (e.g., allograft or xenograft), and orthobiologics.

After the bone growth substance is applied to the fixation assembly, the access device is removed. Absent the retracting force provided by the access device, the patient's tissue generally collapses onto the bone growth substance. The tissue will thereby maintain the position of the bone growth substance adjacent to the fixation assembly. The presence of the bone growth substance advantageously causes bone to grow between the vertebrae and the fixation assembly to form a bridge therebetween.

A fourth MILIF method involves substantially the same procedures performed in connection with the third MILIF method. In particular, one or more implants are positioned in the interbody spaces through an access device, a fixation procedure is performed through the access device, and a further fusion procedure is performed wherein bone growth substance is positioned adjacent the interbody space through the access device. In addition, a decompression procedure is performed through the access device that may include a facetectomy and/or a laminectomy.

A fifth MILIF method involves substantially the same procedures performed in connection with the first MILIF method, except that the fixation is optional. In one embodiment, the fixation is not performed. In addition, a further fusion procedure is performed through the access device wherein bone growth substance is positioned adjacent the interbody space, as discussed above.

A sixth MILIF method is substantially the same as the fifth MILIF method, except that a further fusion procedure is performed through the access device. In particular, an implant is positioned in the interbody space through an access device, a decompression procedure is performed through the access device, and a further fusion procedure is performed whereby bone growth substance is placed adjacent the interbody space through the access device. As discussed above, the decompression procedure may include a facetectomy, a laminectomy, and any other suitable procedure. As with any of the methods described herein, the procedures that make up the sixth MILIF method may be performed in any suitable order. Preferably the decompression procedure is performed before the external fusion procedure.

The foregoing discussion illustrates that a MILIF procedure can include a variety of procedures that can be performed through an access device described herein. In addition, though not expressly set forth herein, any combination of the procedures discussed above, and any other suitable known procedures, may also be combined, as should be understood by one skilled in the art.

C. Other Multi-Level Procedures

While the foregoing procedures have involved interbody fusion, the access devices and systems described herein can be employed in a variety of single level and multi-level procedures (e.g., more than two levels) that do not involve an interbody fusion. For example, a discectomy can be performed through the access devices described herein without implanting an interbody fusion device thereafter, e.g., to remove a herneation. In another embodiment, a discectomy can be performed in more than one interbody space without inserting an interbody fusion device into each interbody space, e.g., to remove multiple herneations. In another embodiment, a single or multi-level decompression procedure can be performed to remove unwanted bone growth.

It will be understood that the foregoing is only illustrative of the principles of the invention, and that various modifications, alterations, and combinations can be made by those skilled in the art without departing from the scope and spirit of the invention. Some additional features and embodiments are described below.

### III. Additional Features and Embodiments of Systems and Methods for Performing Surgical Procedures

FIGS. 67-89 show embodiments of access devices and systems having one or more discrete locations for visualization. FIGS. 67-71 show an access device or retractor 4010 that can be incorporated into a surgical system, such as the system 10. In the illustrated embodiment, the access device 4010 is similar to those access devices hereinbefore described, except as set forth below. The access device 4010 has an elongate body with a distal portion 4028 and a proximal portion 4032. Each of the distal and proximal portions 4028, 4032 define a portion of a passage that extends through the access device 4010. The distal and proximal portions 4028, 4032 may be coupled in any suitable manner, e.g., with a rivet, as discussed below. Although the illustrated embodiment of the access device 4010 has two discrete portions that are coupled in a suitable manner, other access devices embodying features discussed herein can be configured without multiple, discrete portions. Both the proximal and distal portions preferably are made from a rigid, radiolucent material, e.g., one that is visible under fluoroscopy. Both the distal and proximal portions 4028, 4032 preferably have sufficient strength to retract tissue. Examples of materials that may be used and other features that can be incorporated into the access device 4010 are discussed above and in the patents and applications incorporated by reference herein. The distal and proximal portions may be made of different materials.

The proximal portion 4032 preferably is elongated along a longitudinal axis 4036 and has a length along the axis 4036 that is selected based upon the anatomy (e.g., the portion of the spine and the amount of tissue between the skin and the spine) being treated. The length of the proximal portion 4032, and other aspects of the access device 4010, also can be based in part on the individual patient's anatomy. As discussed above, the configuration of at least a portion of the proximal portion 4032 is elongated in at least one direction in a plane perpendicular to the axis 4036. One advantageous arrangement of the proximal portion 4032 provides a generally oblong transverse cross-section. Another advantageous arrangement provides a generally oval transverse cross-section. A proximal portion 4032 with an oblong transverse cross-section is illustrated in FIG. 70, which shows that the transverse cross-section of the proximal portion 4032 is elongated along a line 4040. As will be discussed in more detail below, the line 4040 is along the main axis of expansion of the access device 4010.

There are several advantages to configuring the proximal portion 4032 with an oblong transverse cross-section. Many bone and joint procedures, particularly spinal procedures, are performed at elongated surgical fields. Multi-level procedures, for example, may require providing access to at least a portion of three or more adjacent vertebrae. While symmetrical access could be provided to three or more adjacent vertebrae, much non-adjacent tissue (i.e., tissue not in the immediate vicinity of the structures being treated) would be disrupted, causing greater trauma than necessary to treat the patient. This additional trauma could approach that of open surgery as the length of the surgical field increases. In contrast, the use of an oblong transverse cross-section proximal portion 4032 and access device 4010 lessens, if not minimizes, the amount of non-adjacent tissue that is disrupted. Oblong access from a posterior or posterolateral approach is particularly advantageous in that it provides access to anatomy for a wide variety or procedures, e.g., those that affect the pedicles.

With reference to FIGS. 67-71, the access device 4010 is configured to be coupled with a viewing element to provide or enhance visualization at discrete locations about the longitudinal axis 4036. The access device 4010 comprises a plurality of viewing element passages 4012. The viewing element passages 4012 are located at discrete locations along the perimeter of the proximal portion 4032 of the access device 4010. First and second viewing element passages 4012 preferably are located at first and second discrete locations 4014, 4016 on opposite sides of the access device 4010. Third and fourth viewing element passages 4012 can be located at third and fourth discrete locations 4018, 4020 on one other side of the access device 4010 and preferably are spaced apart. The viewing element passages 4012 and other features of the access device will be discussed further below. The term “passages” is used herein in its ordinary sense and can mean an opening, a path, a channel, or a duct, of any length and configuration, through, over, or along which something may pass, it is a broad term and it can include recesses in a surface, holes in a structure, and defined spaces having short lengths.

Although the access device is illustrated as a four viewing element passage arrangement, other arrangements are possible and contemplated. For example, more or less than four viewing element passages, different locations of passages, different shaped passages, and different configurations of passages could be used in place of or in combination with the illustrated four viewing element passage arrangement. For example, a single passage 4012 could be provided. The viewing element passages are generally smaller than the main access passage of the access device. For example, in some embodiments, the cross sectional area of the main passage can be between about 10 times to about 30 times greater than the cross sectional area of a viewing element passage. In other embodiments, the cross sectional area of the main passage can be less than about 10 times greater, or more than about 30 times greater, than the cross sectional area of a viewing element passage.

As described above, the main passage preferably has a desired general shape, e.g., generally oblong, oval, circular, etc., that may be affected somewhat by the shapes of the viewing element passages, but which generally provides access as desired. The viewing element passages may be, but are not necessarily, integral with the elongate body defining the main passage. As used herein, the term “integral” is a broad term, used in its ordinary sense, and includes being formed together or subsequently joined together, for example, by welding of the passages or other suitable methods. The viewing element passages may be symmetrically disposed relative one or more axes of the access device. In some embodiments, the viewing element passages are not symmetrically disposed relative to one or more axes of the access device. The viewing element passages, in some embodiments, need not extend to the distal end of the access device.

In some embodiments, access devices having one or more viewing element passages are configured to be distally expandable. In some other embodiments, access devices having one or more viewing element passages are not configured to be distally expandable. For example, in some embodiments, an access device has an unexpandable straight main passage with one or more viewing element passages. In some embodiments, a single wall separates the main passage from the viewing element passages. The size of the main passage of the access device preferably is maximized relative to the size of the viewing element passages, in some embodiments. Additional structures, such as partitions or dividing elements, that are positioned within the main passage preferably are minimized or avoided, in some embodiments. The size and positioning of the viewing element passages preferably have a relatively simple construction and act to stabilize the access device. Other embodiments having different arrangements are described below with reference to FIGS. 77-89.

The distal portion 4028 also extends along the longitudinal axis 4036 and comprises a first overlapping portion 4050 and a second overlapping portion 4054. The first overlapping portion 4050 extends between a proximal end 4058 and a distal end 4062 of the distal portion 4028. The second overlapping portion 4054 extends between the proximal end 4058 and the distal end 4062 of the distal portion 4028. The overlapping portions 4050, 4054 overlap each other to create an enclosed space 4066 therebetween. In one embodiment, each of the overlapping portions 4050, 4054 extends along the axis 4036 when the overlapping portions are coupled with the proximal portion 4032 and is formed from a thin, rigid material (such as sheet metal) that is curled into a generally U-shaped structure.

The first and second overlapping portions 4050, 4054 are coupled in a manner that permits expansion of the distal portion 4028 at least at the distal end 4062. The advantages of being able to expand the distal portion 4028 are discussed above. The first and second overlapping portions 4050, 4054 may be configured to be selectively locked or unlocked in one or more states of expansion or contraction. In some embodiments, interaction between the retracted tissue, the access device 4010, and anatomy distal of the distal end 4062 maintains the access device 4010 in the expanded configuration.

In one embodiment, the distal portion 4028 has a slot and guide member arrangement that enables expansion of the distal portion 4028. Corresponding arcuate slots 4070a, 4070b are formed in the first overlapping portion 4050 and the second overlapping portion 4054, respectively. In one embodiment, a guide member, such as a sliding rivet 4074, extends through the corresponding slots 4070a, 4070b thereby coupling the slots. The slots 4070a, 4070b and the rivet 4074 enable the distal portion 4028 to be expanded by allowing the rivet 4074 to slide along the slots while the overlapping portions 4050, 4054 move away from or toward each other. In the illustrated embodiment, a second pair of slots and a corresponding guide member (e.g., a rivet), are provided on the opposite side of the access device 4010 from the slots 4070a, 4070b and the rivet 4074. Thus, two rivets 4074 are provided in corresponding pairs of slots adjacent each edge of the overlapping sections 4050, 4054. This arrangement enables generally linear expansion of the distal portion 4028 along and parallel to the vertical plane containing the line 4040. Another arrangement provides one or more slots on only one side of the access device, which would provide a more two-dimensional expansion near the distal end 4062 of the distal portion 4028.

The distal portion 4028 is configured to be actuatable from an unexpanded configuration to an expanded configuration. The unexpanded configuration is said to be “low-profile” in that the transverse cross-section of the distal portion 4028, particularly at the distal end 4062, is relatively small, e.g., a size suitable for insertion over a dialator. The access device 4010, like the other access devices described hereinabove, is configured to be inserted over a dilating structure, such as a dilator or an obturator. Providing a low-profile distal end 4062 in the unexpanded configuration enables a generally smaller dilating structure to be used, reducing the amount of trauma during insertion. In one embodiment the distal portion 4028 has an oblong cross-section similar to that of the proximal portion 4032 when the distal portion 4028 is in a low profile configuration. The transverse cross-section of the distal portion 4028 in the low profile configuration need not be constant from the distal end 4058 to the proximal end 4062. For example, in one embodiment the transverse cross-section of the distal portion 4028 transitions from generally circular near the distal end 4058 to generally oblong near the proximal end 4062 (e.g., matching the transverse cross-section of the proximal portion 4062 at the distal end of the proximal portion 4062). The distal portion 4028 may also be arranged to transition from a circular cross-section configuration to another non-circular cross-section configuration.

In one embodiment, the distal portion 4028 has at least one notch 4024 provided at its proximal end 4058 that enables expansion of the distal portion 4028 at one or more of the discrete locations where the viewing element passages 4012 of the proximal portion 4032 are defined. In the illustrated embodiment, notches 4024 are provided in the distal portion 4028 adjacent the first and second viewing element passages 4012 at the first and second discrete locations 4014, 4016. In one embodiment, the notch 4024 enables the distal portion 4028 to expand from an initial position without interfering with the viewing element passages 4012. In the illustrated embodiment, notches 4024 are provided in the distal portion 4028 to enable expansion over viewing element passages 4012 located on opposite sides of the access device 4010. This arrangement enables generally linear expansion of the distal portion 4028 along and parallel to the vertical plane containing the line 4040.

Although the access device is illustrated as a two notch arrangement, other arrangements are possible and contemplated. For example, more or less than two notches, different locations of notches, different shaped notches, and different configurations of openings or flexible materials could be used in place of or in combination with the illustrated two notch arrangement. Other embodiments having different arrangements are described below with reference to FIGS. 80-89.

In one application, the access device 4010 is used to provide minimally invasive access to the spine for a spinal procedure, such as a one-level or a multi-level procedure. The patient is positioned prone on a radiolucent table and draped for posterior spinal surgery. The location of the spine anatomy to be treated is identified, e.g., via fluoroscopy. In one embodiment, adjacent pedicles on one side of the mid-line of the spine are located. Thereafter, an incision is made through the skin above the adjacent pedicles. An incision of about 30-40 mm in length is made between two adjacent pedicles where a single level procedure (one involving two adjacent vertebrae) is to be performed. Where a two-level procedure (one involving three vertebrae) is to be performed, an incision of 40-50 mm in length is made.

Thereafter a dilating device, such as a series of dilators, is inserted into the incision to enlarge the incision. It may be desirable to use round or oblong dilators. Preferably the last dilator has an outer profile that matches the unexpanded inner profile of the access device 4010. In a single level procedure, a 5 mm dilator is first inserted through the center of the skin incision and is docked on the lateral aspect of the superior facet. In a two-level procedure, a 5 mm dilator is first advanced through the center of the incision and is docked on the mamillo-accessory ridge of the middle pedicle. Placement of the 5 mm dilator may be verified by fluoroscopy. Subsequently, progressively larger dilators are inserted over each other. After a larger dilator is inserted, the next-smaller dilator is normally removed. One or more of the dilators, a cobb device, or even one of the surgeon's fingers may also be used to probe and to dissect soft tissue to ease expansion of the access device 4010, as discussed below. Placement of the final dilator may be verified by fluoroscopy.

Thereafter, the access device 4010 is advanced to the anatomy to be treated. As discussed above, the access device 4010 may be maintained in the unexpanded configuration prior to deployment by a sleeve (not shown) deployable by a string (not shown). In one technique, the access device-sleeve-string assembly is inserted into the incision and positioned so that the string faces the mid-line of the spine. Thereafter the string is withdrawn, releasing the sleeve from the access device 4010. After the sleeve is released from the access device 4010, the access device 4010 is free to expand and to be expanded. In some embodiments, the sleeve remains positioned about the access device such that tissue intrusion between the viewing element passage and the notch portion is limited or prevented. The access device 4010 resiliently expands in some embodiments and in some applications. Further expansion of the access device 4010 may be achieved by inserting and articulating an expander tool, such as the expander tool 200 discussed above. The expansion and location of the access device 4010 may be confirmed by fluoroscopy.

After the access device 4010 is expanded, various procedures may be performed on the spine (or other joint or bone segment). As discussed above, these procedures may be performed with much less trauma than that associated with more invasive surgery, such as open surgery. After the procedures are complete, the access device 4010 may be unexpanded and removed.

FIGS. 72-76 show an access assembly 4000 that can be incorporated into a surgical system, such as the system 10. The access assembly 4000 includes the access device 4010 coupled with a mount fixture 4100. The mount fixture 4100 may be coupled with a support arm (not shown), such as the support arm A discussed above. In the illustrated embodiment, the mount fixture 4100 comprises an access device support portion 4102 and a viewing element support portion 4104. The access device 4010 preferably is provided with an oblong transverse cross-section near the proximal end thereof, i.e., the end that is coupled with the access device support portion 4102 of the mount fixture 4100. As described above, the oblong shaped cross-section of the access device 4010 is particularly beneficial for surgical procedures performed at an elongated surgical field, such as two level pedicle screw fixation. A guide fixture 4106 is coupled with the viewing element support portion 4104 of the mount fixture 4100. The guide fixture 4106 is configured to be coupled with a viewing element 4108, which may be any of those discussed hereinabove, or any other suitable viewing element. In the illustrated embodiment, the viewing element 4108 is an endoscope. In some embodiments, the guide fixture 4106 is capable of vertical adjustment of the viewing element 4108, e.g., the guide fixture 4106 may comprise a jack.

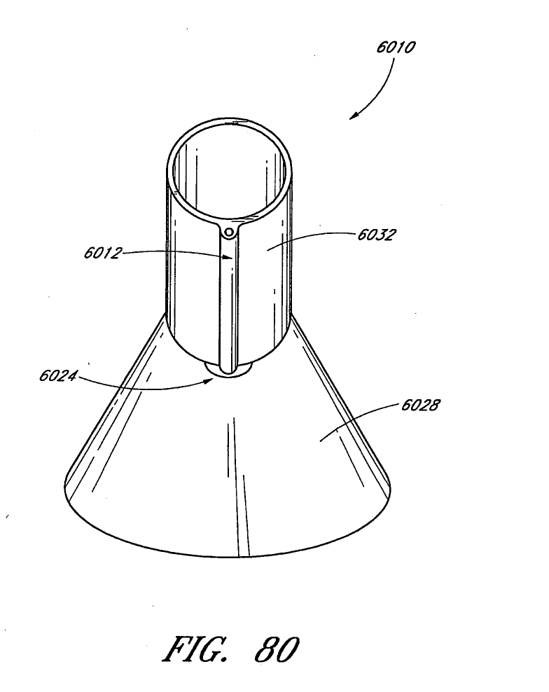

With reference to FIG. 76, the access device 4010 comprises a plurality of viewing element passages 4012. The viewing element passages 4012 are located at discrete locations along the perimeter of a proximal portion of the access device 4010. The viewing element passages 4012 preferably are configured (e.g., are sized) to receive a viewing element 4108. In the illustrated embodiment, the passages 4012 are formed integrally (e.g., a one-piece construction, such as by injection molding or casting) within a wall of the proximal portion of the access device 4010. In other embodiments, passages are formed separately and are located on an interior surface of a wall of an access device. In still other embodiments, passages are located on an exterior surface of the wall of the access device 4010. The passages 4012, may advantageously protect the viewing element 4108. The passages 4012 are at least partially separated from a main central passage of the access device 4010. While the passages 4012 are described as viewing element passages, it will be apparent to one of skill in the art that the passages 4012 can be provided for any purpose. For example, in some embodiments, the passages 4012 may provide access to a surgical site for suction, irrigation, instrumentation, visualization, or any other reason. Thus, passages 4012 are not intended to be limited to receiving viewing elements, but can be arranged or configured for other suitable uses.

The mount fixture 4104 and the guide fixture 4106 advantageously are configured to introduce the viewing element 4108 into the access device 4010 at discrete locations. In the illustrated embodiment, the mount fixture 4104 and the guide fixture 4106 are configured to enable the viewing element 4108 to be positioned at least partially within the viewing element passages 4012 of the access device 4010.

With reference to FIGS. 76 and 76A, the access device support portion 4102 of the mount fixture 4100 is configured to be coupled to a proximal portion of the access device 4010. In the illustrated embodiment, the access device support portion 4102 has a support ring 4110 and an extension arm 4112. The extension arm 4112 preferably is coupled to the viewing element support portion 4104 and a support arm (not shown), such as the support arm A discussed above. The support ring 4110 preferably is shaped to fit over, or rest on top of, a proximal portion of the access device 4010. The support ring 4110 in the illustrated embodiment has an oblong, generally oval shape. FIG. 76A is a cross-sectional view of the support ring 4110. Side portions of the support ring 4110 preferably are sized to fit over the proximal portion of the access device 4010. The support ring 4110 could be coupled to the access device 4010 with a suitable coupling device, such as, for example, set screws (not shown). An opening 4114 is provided at each of four discrete locations on the top surface 4115 of the access device support portion 4102. When the access device support portion 4102 is coupled with the access device 4010, each opening 4114 is configured to be aligned with the corresponding viewing element passage 4012 of the access device 4010. The openings 4114 and the viewing element passages 4012 preferably are configured to receive at least a portion of the viewing element 4108.

With reference to FIG. 76, the viewing element support portion 4104 of the mount fixture 4100 is configured to be coupled to the access device support portion 4102 and the support arm (not shown). In the illustrated embodiment, the viewing element support portion 4104 has a support mount 4116 and an extension arm 4118. The extension arm 4118 preferably is coupled to the access device support portion 4102 and the support arm, as discussed above. As shown in FIG. 76, the extension arm 4118 has a slot 4120 for receiving at least a portion of the extension arm 4112 of the access device support portion 4102. The extension arm 4118 of the viewing element support portion 4104 and the extension arm 4112 of the access device support portion 4102 each preferably define an opening 4122, 4124, respectively, at coupling locations on their proximal portions. The openings 4122, 4124 are configured such that when the extension arm 4112 is located within the slot 4120 of the extension arm 4118, the openings 4122, 4124 are aligned. The support arm can be coupled to the mount fixture 4100 at the coupling locations. A portion of the support arm assembly, such as, for example, a pin, can extend through the openings 4122, 4124 to couple the support arm and the mount fixture 4100 together.

The viewing element support portion 4104 preferably is shaped to fit over the access device support portion 4102, as is shown in FIGS. 72-75. In the illustrated embodiment, the support mount 4116 of the viewing element support portion 4104 has an oblong, generally oval shape defining an interior space. The support mount 4116 has an end portion 4126 opposite the extension arm 4118. The end portion 4126 has generally flat interior and exterior surfaces. The interior surface defines a notch 4128 in the end portion 4126. The support mount 4116 also has first and second side portions 4130, 4132, respectively, adjacent the end portion 4126, that each have generally flat interior and exterior surfaces. The interior surfaces define notches 4134, 4136 in the first and second side portions 4130, 4132, respectively. When the viewing element support portion 4104 is coupled with the access device support portion 4102, there is a gap or space 4138 created between an interior surface of the viewing element support portion 4104 and an exterior surface of the access device support portion 4102, as shown in FIG. 74. The gap or space 4138 preferably allows the guide fixture 4106 to be coupled to the viewing element support portion 4104. As described further below, at least a portion of the guide fixture 4106 preferably extends into the space 4138 between the access device support portion 4102 and the viewing element support portion 4104.

The guide fixture 4106 is configured to be coupled to the viewing element support portion 4116 of the mount fixture 4100. In the illustrated embodiment, a first slot 4140 is defined in a bottom surface of the guide fixture 4106. The slot 4140 preferably is sized and configured such that the guide fixture can be placed over and supported by a portion of the viewing element support portion 4104. The guide fixture 4106 can be coupled with at least one of the end portion 4126, the first side portion 4130, and the second side portion 4132 of the viewing element support portion 4104. The slot 4140 of the guide fixture 4106 preferably is inserted over the viewing element support portion 4104 at one or more of the notched portions 4126, 4130, 4132.

The guide fixture 4106 has a viewing element coupling portion. In the illustrated embodiment, the viewing element coupling portion includes a guide channel 4142 and an adjustment device 4144. The guide fixture 4106 can be placed in any one of a plurality of positions relative the viewing element support portion 4104. The guide fixture 4106 preferably can be placed in a first position on the end portion 4126, in a second position on the first side portion 4130, and in a third position on the first side portion 4130. The guide channel 4142 preferably is aligned with a first opening 4114a of the access device support portion 4102 and a first viewing element passage 4012 when the guide fixture 4106 is in the first position relative the viewing element support portion 4104. In the first position, a viewing element 4108 can extend through the first opening 4114a to a position, preferably within the access device, for visualization of the surgical location from a first viewing perspective, e.g., a perspective from a first end portion of the access device of the surgical area defined within the access device. The guide channel 4106 preferably is aligned with a second opening 4114b of the access device support portion 4102 and a second viewing element passage 4012 when the guide fixture 4106 is in the second position relative the viewing element support portion 4104. In the second position, a viewing element 4108 can extend through the second opening 4114b to a position, preferably within the access device, for visualization of the surgical location from a second viewing perspective, e.g., a perspective from a first side portion of the access device near the first end portion of the access device. The guide channel 4106 preferably is aligned with a third opening 4114c of the access device support portion 4102 and a third viewing element passage 4012 when the guide fixture 4106 is in the third position relative the viewing element support portion 4104. In the third position, a viewing element 4108 can extend through the third opening 4114c to a position, preferably within the access device, for visualization of the surgical location from a third viewing perspective, e.g., a perspective from a first side portion of the access device farther from the first end portion of the access device than the perspective from the second position. In the illustrated embodiment, at least a portion of the viewing element, e.g., endoscope, can extend through the guide channel 4142 and openings 4114a, 4114b, 4114c into the viewing element passages 4012 of the access device 4010 to enable the user to see within the access device 4010.

In the illustrated embodiment, the adjustment component 4144, e.g., a jack, comprises a dial 4146 coupled with an elevation member 4148 adapted to support the viewing element 4108. Rotation of the dial 4146 causes the elevation member 4148 to move up or down relative the guide fixture 4106. In the illustrated embodiment, the endoscope is configured to be coupled to the guide fixture 4106. The endoscope can be raised or lowered relative the guide fixture 4106 by the elevation member 4148, which is actuated by turning the dial 4146. For example, rotation of the dial 4146 can be used to provide a zoom feature for the endoscope. Rotation of the dial 4146 to lower the endoscope can cause the endoscope to zoom in. Rotation of the dial 4146 to raise the endoscope can cause the endoscope to zoom out.

The guide fixture 4106 preferably is securely coupled with the mount fixture 4100 so that the guide fixture 4106 will not be dislodged inadvertently during a procedure. However, the engagement also is such that a user of the access assembly 4000 can disengage the guide fixture 4106 and viewing element 4108 and reposition them at any of the other discrete locations during a procedure. Although the coupling of the mount fixture 4100 and the guide fixture 4106 is illustrated as a slot arrangement, other arrangements are possible and contemplated. For example, a plurality of slots, couplers of other shapes (e.g., one or more pins and holes, tongues and slots of different shapes, etc.), and clamp devices could be used in place of or in combination with the illustrated slot arrangement.

In use, the access device support portion 4102 of the mount fixture 4100 is coupled to the access device 4010. The openings 4114 in the access device support portion 4102 are aligned with the viewing element passages 4012 of the access device 4010. The guide fixture 4106 is coupled to the viewing element support portion 4104 of the mount fixture 4100 at one of the discrete viewing element locations. The guide channel 4142 is aligned with at least one of the openings 4114 in the access device support portion 4102 and at least one of the viewing element passages 4012 of the access device 4010. The viewing element 4108 is coupled to the guide fixture 4106. At least a portion of the viewing element 4108 extends through the guide channel 4142, at least one opening 4114 of the access device support portion 4102, and at least one of the viewing element passages 4012 of the access device 4010.

FIGS. 77-79 show another embodiment of an access device 5010 and access assembly 5000 that are similar to those hereinbefore described, except as set forth below. With reference to FIGS. 77-79, the access device 5010 comprises a plurality of viewing element passages 5012. The viewing element passages 5012 are located at discrete locations about the perimeter of a proximal portion of the access device 5010. First and second viewing element passages 5012 preferably are located at first and second discrete locations spaced apart on a first side of the access device 5010. Third and fourth viewing element passages 5012 are located at third and fourth discrete locations spaced apart on a second side of the access device 5010. In the illustrated embodiment, the first side is opposite the second side.

Although the access device 5010 is illustrated as a four viewing element passage arrangement, other arrangements are possible and contemplated. For example, more or less than four viewing element passages, different locations of passages, different shaped passages, and different configurations of passages could be used in place of or in combination with the illustrated four viewing element passage arrangement.

The access assembly 5000 includes the access device 5010 coupled with a mount fixture 5100. The mount fixture 5100 may be coupled with a support arm (not shown), such as those discussed above. In the illustrated embodiment, the mount fixture 5100 comprises an access device support portion 5102 and a viewing element support portion 5104. The access device 5010 preferably is provided with an oblong transverse cross-section near the proximal end thereof, which is coupled with the access device support portion 5102 of the mount fixture 5100. Coupled with the viewing element support portion 5104 of the mount fixture 5100 is a guide fixture 5106. The guide fixture 5106 is configured to be coupled with a viewing element 5108, which may be any of those discussed hereinabove, or any other suitable viewing element. In the illustrated embodiment, the viewing element 5108 is an endoscope. The access device 5010 comprises a plurality of viewing element passages 5012, as discussed above.

The mount fixture 5104 and the guide fixture 5106 advantageously are configured to introduce the viewing element 5108 into the access device 5010 at discrete locations. In the illustrated embodiment, the mount fixture 5104 and the guide fixture 5106 are configured to enable a viewing element 5108 to be positioned at least partially within the viewing element passages 5012 of the access device 5010.

The access device support portion 5102 of the mount fixture 5100 is configured to be coupled to a proximal portion of the access device 5010. In the illustrated embodiment, the access device support portion 5102 has a support ring 5110 and an extension arm 5112. The support ring 5110 preferably is shaped to fit over a proximal portion of the access device 5010. The support ring 5110 in the illustrated embodiment has an oblong, generally oval shape defining a central space. The support ring 5110 is coupled to the access device 5010 with a suitable coupling device, such as, for example, set screws (not shown). Openings 5114 are provided at four discrete locations on the top surface of the access device support portion 5102. When the access device support portion 5102 is coupled with the access device 5010, each opening 5114 is configured to be aligned with the corresponding viewing element passage 5012 of the access device 5010. The openings 5114 and the viewing element passages 5012 preferably are configured to receive at least a portion of a viewing element 5108.

The extension arm 5112 of the access device support portion 5102 preferably is coupled to a support arm (not shown), such as those discussed above. The extension member 5112 preferably defines an opening 5124 at a coupling location on its proximal portion. The support arm can be coupled to the mount fixture 5100 at the coupling location. A portion of the support arm assembly, such as, for example, a pin, can extend through the opening 5124 to couple the support arm and the mount fixture 5100 together.

The extension arm 5112 preferably is coupled to the viewing element support portion 5104. The extension arm 5112 preferably is rotatably coupled to the viewing element support portion 5104. In the illustrated embodiment, the extension arm 5112 has a curved slot 5120 defined between the body of the extension arm 5112 and a post 5122 extending upward from the extension arm 5112. The slot 5120 and post 5122 arrangement of the extension arm 5112 is configured to receive and be rotatably coupled to the viewing element support portion 5104 of the support fixture 5100.

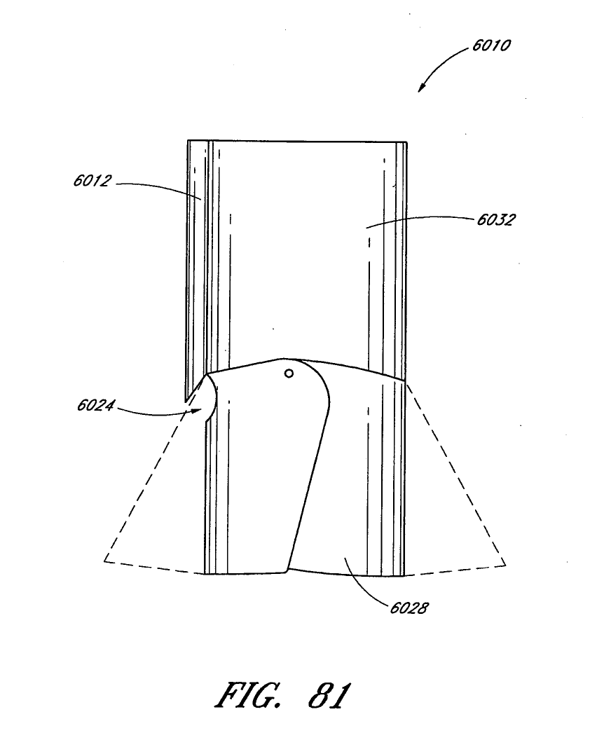

The viewing element support portion 5104 of the mount fixture 5100 is configured to be coupled to the access device support portion 5102. In the illustrated embodiment, the viewing element support portion 5104 has a support mount 5116 and an extension element 5118. As discussed above, the extension element 5118 preferably is coupled to the access device support portion 5102. As shown in FIG. 78, the extension element 5118 defines an opening 5126 at one end. The extension element 5118 of the viewing element support portion 5104 is configured such that a portion of the extension element 5118 can extend into the slot defined in the extension arm 5112 of the access device support portion 5102, and such that the post 5122 of the extension arm 5112 extends into the opening 5126 in the extension element 5118 for rotatably coupling the viewing element support portion 5104 to the access device support portion 5102.

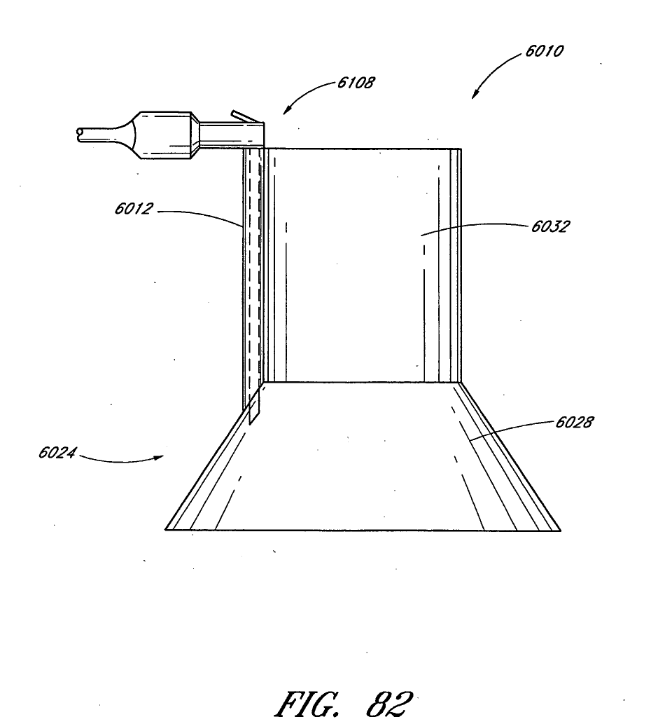

The viewing element support portion 5104 preferably is positioned above the access device support portion 5102. The support mount 5116 of the viewing element support portion 5104 in the illustrated embodiment has a generally trapezoidal shape. The support portion 5104 may have a generally triangular shape. Support mounts in other embodiments can have other suitable shapes. As shown in the illustrated embodiment, the support mount 5116 is provided with a plurality of holes 5128. The holes 5128 are provided at four discrete locations in support mount 5116 of the viewing element support portion 5104. With reference to FIG. 79, the support mount 5116 preferably is rotatable between a first viewing position and a second viewing position shown in dashed line. When the viewing element support portion 5104 is placed in a first position relative the access device 5010, one or more holes 5128 of the viewing element support portion 5104 are aligned with one or more of the corresponding viewing element passages 5012 of the access device 5010. When the viewing element support portion 5104 is placed in a second position relative the access device 5010 and/or the access device support portion 5102, one or more holes 5128 of the viewing element support portion 5104 are aligned with one or more of the corresponding viewing element passages 5012 of the access device 5010. The viewing element support portion 5104 preferably is configured such that it does not significantly obstruct a passage 5066 of the access device when placed in the first position or the second position. Also, the viewing element support portion 5104 eliminates the additional structure surrounding the access device support portion 5102, as in the embodiment of FIGS. 72-76. This reduces the amount of supporting structure that could obstruct the access of the surgeon. The holes 5128 and the viewing element passages 5012 preferably are configured to receive at least a portion of a viewing element 5108.

The guide fixture 5106 is configured to be placed on the viewing element support portion 5104 of the mount fixture 5100. In the illustrated embodiment, a bottom surface of the guide fixture 5106 is located adjacent an upper surface of the viewing element support portion 5104. The guide fixture 5106 has a viewing element coupling portion. In the illustrated embodiment, the viewing element coupling portion includes a guide channel 5142 and an adjustment system 5144.

The guide fixture 5106 can be placed in one of a plurality of positions relative the viewing element support portion 5104. The guide channel 5142 is configured such that it is aligned with a first opening 5128 of the viewing element support portion 5104 and a first viewing element passage 5012 of the access device 5010 when the guide fixture 5106 is in a first position relative the viewing element support portion 5014. The guide channel 5142 is configured such that it is aligned with a second opening 5128 of the viewing element support portion 5104 and a second viewing element passage 5012 of the access device 5010 when the guide fixture 5106 is in a second position relative the viewing element support portion 5104. The guide channel 5142 is configured such that it is aligned with a third opening 5128 of the viewing element support portion 5104 and a third viewing element passage 5012 of the access device 5010 when the guide fixture 5106 is in a third position relative the viewing element support portion 5104. The guide channel 5142 is configured such that it is aligned with a fourth opening 5128 of the viewing element support portion 5104 and a fourth viewing element passage 5012 of the access device 5010 when the guide fixture 5106 is in a fourth position relative the viewing element support portion 5104. In the illustrated embodiment, at least a portion of the endoscope can extend through the guide channel 5142, holes 5128, and openings 5114 into the viewing element passages 5012 of the access device 5010 to enable the user to see within the access device 5010.

In the illustrated embodiment, the adjustment system 5144 comprises a dial 5146 coupled with an elevation member 5148 adapted to support the viewing element 5108 in a manner similar to that described above with reference to the adjustment component 4144.

The guide fixture 5106 preferably is securely coupled with the mount fixture 5100 so that the guide fixture 5106 will not be dislodged inadvertently during a procedure. However, the engagement also is such that a user of the access assembly 5000 can disengage the guide fixture 5106 and the viewing element 5018 and reposition them at other discrete locations during a procedure.

In use, the access device support portion 5102 of the mount fixture 5100 is coupled to the access device 5010. Openings 5114 in the access device support portion 5102 are aligned with the viewing element passages 5012 of the access device 5010. The viewing element support portion 5104 is placed in either the first position or the second position, such that one of more holes 5128 of the viewing element support portion 5104 are aligned with one or more viewing element passages 5012 of the access device 5010. The guide fixture 5106 is supported on the viewing element support portion 5104 of the mount fixture 5100 at one of the discrete viewing element locations. The guide channel 5142 is aligned with at least one of the holes 5128 of the viewing element support portion 5104, at least one of the openings 5114 in the access device support portion 5102, and at least one of the viewing element passages 5012 of the access device 5010. The viewing element 5108 is coupled to the guide fixture 5106. At least a portion of the viewing element 5108 extends through the guide channel 5142, at least one of the holes 5128 of the viewing element support portion 5104, at least one opening 5114 of the access device support portion 5102, and at least one of the viewing element passages 5012 of the access device 5010.

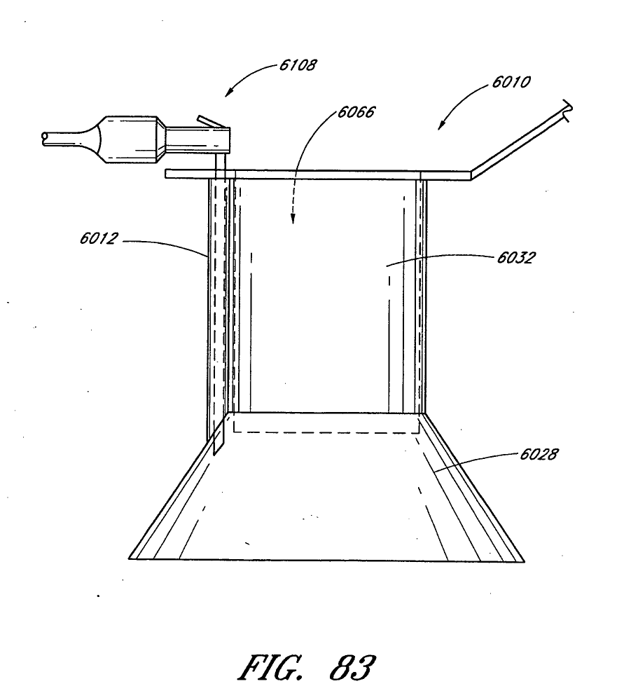

FIGS. 80-89 show other embodiments of access devices and systems that are similar to those hereinbefore described, except as set forth below. With reference to FIG. 80-81, an access device 6010 comprises a single visualization passage 6012. As shown in the illustrated embodiment, the distal portion 6028 of the access device has a notch 6024 provided at a proximal end thereof that enables expansion of the distal portion 6028 at the location where the viewing element passage 6012 is defined. In the illustrated embodiment, the access device 6010 is provided with a generally circular transverse cross-section at a proximal portion 6032 thereof. As shown in FIG. 82, at least a portion of a viewing element 6108, such as, for example, an endoscope or a light source, can be inserted through the viewing element passage 6012 into the access device 6010.

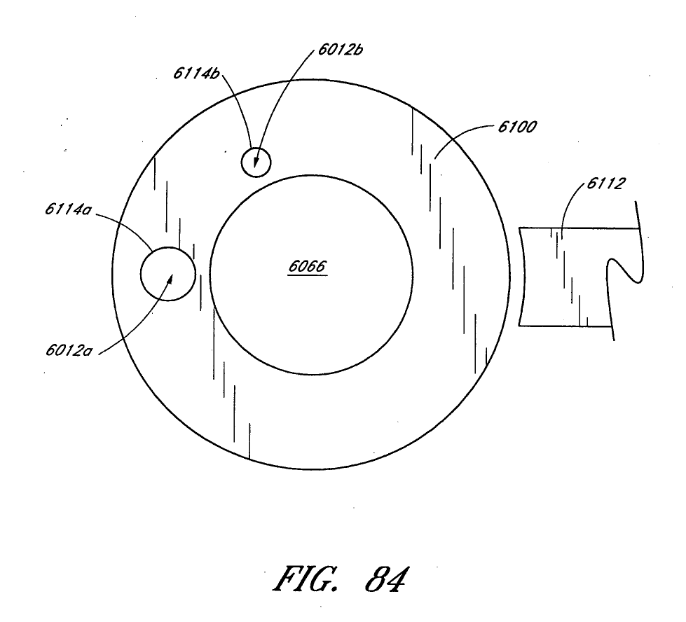

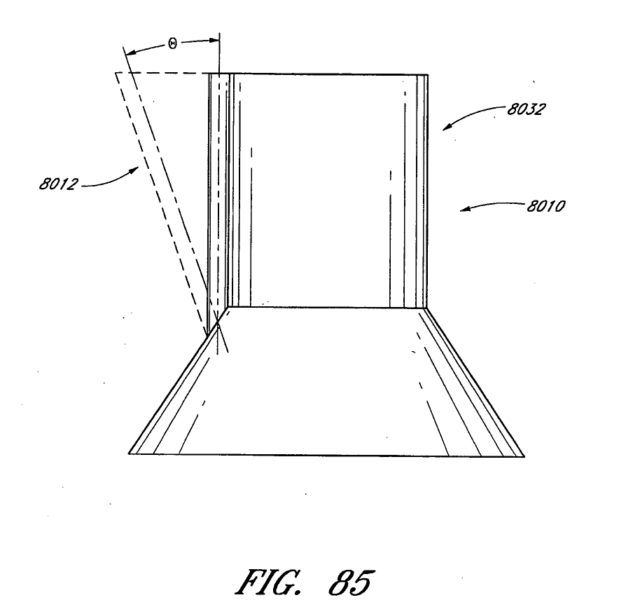

FIGS. 83-84 show an access assembly 6000 that includes the access device 6010 coupled with a mount fixture 6100. The mount fixture 6100 may be coupled with a support arm (not shown), such as those discussed above. In the illustrated embodiment, the mount fixture 6100 is coupled to the access device 6010 and the viewing element 6108. The access assembly 6000 can provide for direct visualization, e.g., using a microscope, loupes, or the unaided eye, of a surgical location through the main central channel 6066 of the access device 6010. The access assembly 6000 can also provide for visualization via a viewing element 6108, such as with an endoscope, through the viewing passage 6012 without significant obstruction of the central channel 6066 of the access device 6010. The access assembly 6000 provides for quick and easy transition from one visualization mode to another. The central channel 6066 of the access device 6010 preferably is from about 10 mm to about 40 mm in diameter. The viewing element passage 6012 of the access device 6010 preferably is from about 2 mm to about 8 mm in diameter.

With reference to FIG. 84, some embodiments of access devices and systems have a plurality of passages 7012 arranged along the perimeter of an access device 7010. A first passage 7012a is arranged along the perimeter and is configured for visualization. A second passage 7012b is arranged along the perimeter and is configured for suction, irrigation, and/or instrumentation. In other embodiments, one or more passages 7012 are configured for any one of visualization, suction, irrigation, and/or instrumentation. As shown in FIG. 84, a fixture 7100 has a plurality of holes 7114a, 7114b corresponding to the plurality of passages 7012a, 7012b defined in the access device 7010. Additionally, a connection 7112 is provided to a support arm (not shown) to stabilize the access device 7010. The connection 7112 preferably is rotatable relative to the mount fixture 7100 and the access device.

As shown in FIG. 85, in some embodiments a viewing element passage 8012 can be angled relative a proximal portion 8032 of an access device 8010. Viewing element passages 8012 can be angled from about 0 degrees to more than about 25 degrees relative the proximal portion 8032 of the access device 8010. Angled viewing element passages 8012 can, in some embodiments, provide for increased visualization of the surgical space.

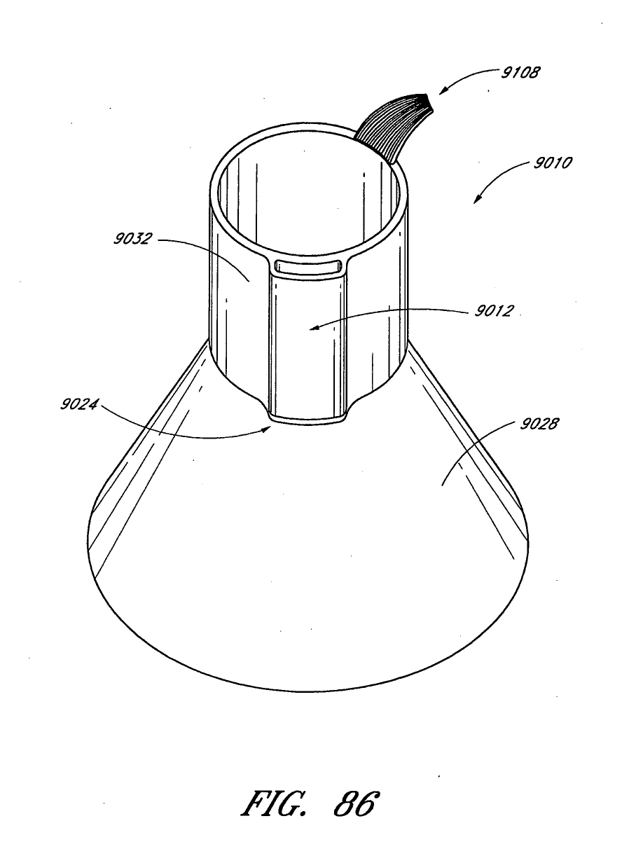

With reference to FIG. 86, in another embodiment, an access device 9010 comprises one or more viewing element passages 9012. The viewing element passage 9012 has a generally oblong shaped cross section. The viewing element passage 9012 preferably is configured to receive a viewing element 9108. In the illustrated embodiment, the access device 9010 is constructed of a light transmitting material, such as, for example, an acrylic or glass-like material. With reference to FIG. 86, in some embodiments a viewing element 9108, such as a light transmitting device, can be coupled directly to a proximal portion 9032 of the access device 9010. The distal portion 9028 of the access device 9010 has at least one notch 9024 provided at a proximal portion that enables expansion of the distal portion 9028 at the location where the viewing element passage 9012 is defined. In the illustrated embodiment, the access device 9010 is provided with a generally circular cross-section.

The various devices, methods and techniques described above provide a number of ways to carry out the invention. Of course, it is to be understood that not necessarily all objectives or advantages described may be achieved in accordance with any particular embodiment described herein. Also, although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein.

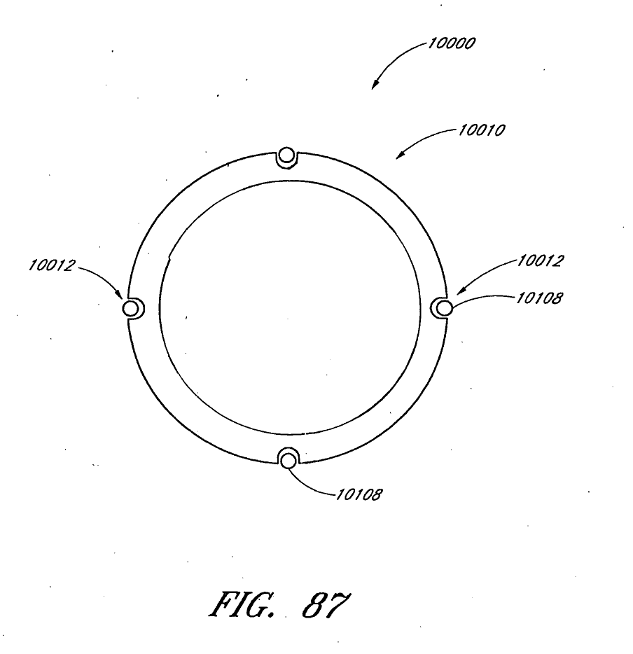

FIGS. 87-89 illustrate additional embodiments of access devices and access assemblies. FIG. 87 illustrates a cross-sectional view of an access device 10010 and access assembly 10000 that are similar to those hereinbefore described, except as set forth below. With reference to FIG. 87, the access device 10010 comprises a plurality of viewing element passages 10012. The viewing element passages 10012 are located at discrete locations about the perimeter of a proximal portion of the access device 10010. Preferably, at least a portion of the length of the access device 10010 defines a working channel. The viewing element passages 10012, as shown in FIG. 87, preferably are located outside of the working channel of the access device 10010. In some embodiments, a proximal portion of the access device 10010 has a cross section as illustrated in FIG. 87. In some embodiments, the entire length of the access device 10010 has a cross section as illustrated in FIG. 87. In some embodiments, the access device 10010 can have an oblong transverse cross-section near a proximal end thereof. In some embodiments, the access device 10010 can have an oblong transverse cross-section the entire length of the access device 10010. In some embodiments a sleeve or sheath can be positioned about the access device to at least partially define the viewing element passages.

The access device 10010 is configured to be coupled with one or more viewing elements 10108, which may be any of those discussed hereinabove, or any other suitable viewing element. In the illustrated embodiment, the viewing elements 10108 are endoscopes. Although multiple viewing elements 10108 are shown, it is anticipated that normally a single viewing element 10108 will be used. Although the access device 10010 is illustrated as a four viewing element passage arrangement, other arrangements are possible and contemplated. For example, more or less than four viewing element passages, different locations of passages, different shaped passages, and different configurations of passages could be used in place of or in combination with the illustrated four viewing element passage arrangement.

FIG. 88 illustrates an access device 11010 and access assembly 11000 that are similar to those hereinbefore described, except as set forth below. With reference to FIG. 88, the access device 11010 comprises a plurality of viewing element passages 11012. The viewing element passages 11012 are located at discrete locations in a distal portion of the access device 11010. Preferably, at least a portion of the length of the access device 11010 defines a working channel. The viewing element passages 11012, as shown in FIG. 88, preferably are located outside of the working channel of the access device 11010. Although the access device 11010 is illustrated as a two viewing element passage arrangement, other arrangements are possible and contemplated. For example, more or less than two viewing element passages, different locations of passages, different shaped passages, and different configurations of passages could be used in place of or in combination with the illustrated two viewing element passage arrangement.

The access assembly 11000 includes the access device 11010 coupled with a mount fixture 11100. The mount fixture 11100 may be coupled with a support arm (not shown), such as those discussed above. In the illustrated embodiment, the mount fixture 11100 comprises an access device support portion 11102 and a viewing element support portion 11104. The access device 11010 preferably is provided with an oblong transverse cross-section near a central portion thereof, which is coupled with the access device support portion 11102 of the mount fixture 11100. The viewing element support portion 11104 of the mount fixture 11100 is configured to be coupled with a viewing element 11108, which may be any of those discussed hereinabove, or any other suitable viewing element. The viewing element support portion 11104 of the mount fixture preferably comprises guide elements 11106 for guiding the viewing element 11108 to the viewing element passages 11012. In the illustrated embodiment, the viewing element 11108 is an endoscope.

The mount fixture 11100 is advantageously configured to introduce the viewing element 11108 into the access device 11010 at discrete locations in a distal portion of the access device 11010. In the illustrated embodiment, the mount fixture 11100 is configured to be coupled to the access device 11010 at an intermediate location between the proximal end and the distal end of the access device 11010. The guide elements 11106 can include a tip configured to atraumatically displace tissue as the mount fixture 11100 is manipulated over the access device 11010 and through the tissue. The viewing element 11108 preferably is positioned at least partially within the viewing element passages 11012 in the distal portion of the access device 11010.

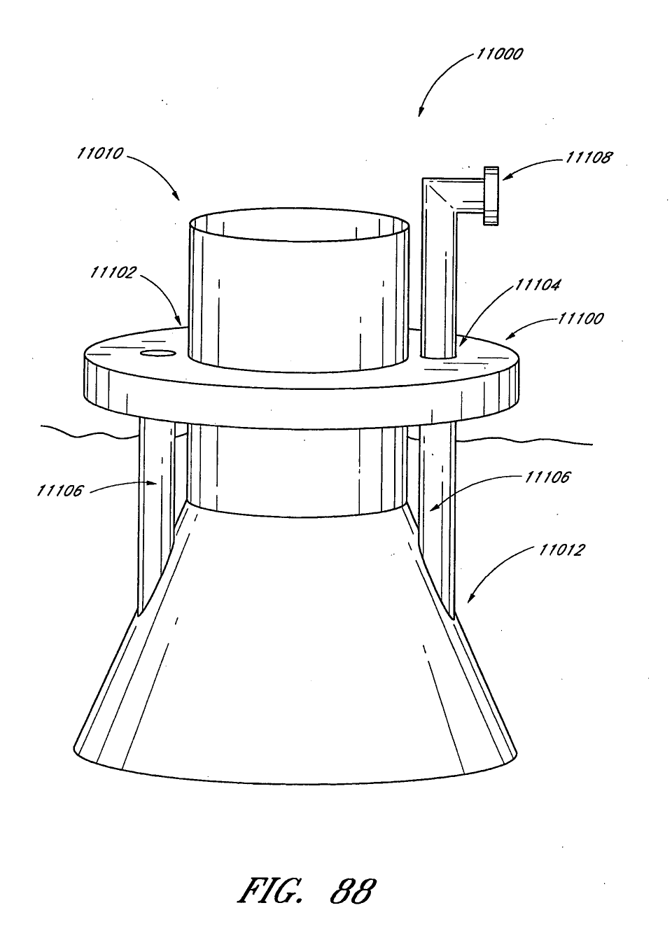

FIG. 89 illustrates an access device 12010 and access assembly 12000 that are similar to those hereinbefore described, except as set forth below. With reference to FIG. 89, the access device 12010 comprises a plurality of viewing element passages 12012. The viewing element passages 12012 are located at discrete locations in an intermediate portion of the access device 12010, between the proximal end and the distal end of the access device 12010. The viewing element passages 12012, as shown in FIG. 89, provide access to the working channel of the access device 12010. Although the access device 12010 is illustrated as a two viewing element passage arrangement, other arrangements are possible and contemplated. For example, more or less than two viewing element passages, different locations of passages, different shaped passages, and different configurations of passages could be used in place of or in combination with the illustrated two viewing element passage arrangement.

The access assembly 12000 includes the access device 12010 coupled with a viewing element 12108. The access device 12010 preferably is provided with an oblong transverse cross-section near an intermediate portion thereof, which is coupled with the viewing element 12108, which may be any of those discussed hereinabove, or any other suitable viewing element. In the illustrated embodiment, the viewing element 12108 is a fiber optic light source. The viewing element passages 12012 are advantageously configured to introduce the viewing element 12108 into the access device 12010 at discrete locations in an intermediate portion of the access device 12010. In the illustrated embodiment, the viewing element 12108 is configured to be coupled to the access device 12010 at an intermediate location between the proximal end and the distal end of the access device 12010. The viewing element 12108 preferably is positioned at least partially within the viewing element passages 12012 in the intermediate portion of the access device 12010.

FIG. 90 shows another embodiment of an access assembly 13000 that can be incorporated into a surgical system, such as the system 10. The access assembly 13000 includes an access device 13010 coupled with a mount fixture 13100. The access assembly 13000 is similar to the access assemblies 4000 and 5000, shown in FIGS. 72-79, except as described below. The mount fixture 13100 may be coupled with a support arm 13101, such as the support arm A discussed above. In the illustrated embodiment, the mount fixture 13100 comprises an access device support portion 13102 and a viewing element support portion 13104. A guide fixture 13106 is coupled with the viewing element support portion 13104 of the mount fixture 13100. The guide fixture 13106 is configured to be coupled with a viewing element 13108, which may be any of those discussed hereinabove, or any other suitable viewing element. The access device 13010 comprises at least one external viewing element passage 13012. The viewing element passage 13012 is located at a discrete location along the perimeter of at least a proximal portion of the access device 13010. The viewing element passage 13012 preferably is configured (e.g., are sized) to receive the viewing element 13108. The mount fixture 13104 and the guide fixture 13106 advantageously are configured to introduce the viewing element 13108 into the access device 13010 at a discrete location. In the illustrated embodiment, the mount fixture 13104 and the guide fixture 13106 are configured to enable the viewing element 13108 to be positioned at least partially within the viewing element passages 13012 of the access device 13010. The access device support portion 13102 of the mount fixture 13100 is configured to be coupled to a proximal portion of the access device 13010. The proximal portion of the access device 13010 preferably comprises a flange 13013.

In the illustrated embodiment, the access device support portion 13102 has a support ring 13110 and an extension arm 13112. The extension arm 13112 preferably is coupled to the viewing element support portion 13104 and/or the support arm 13101. The viewing element support portion 13104 of the mount fixture 13100 is configured to be coupled to the access device support portion 13102 and/or the support arm 13101. In the illustrated embodiment, the viewing element support portion 13104 has a support mount 13116 and an extension arm 13118. The extension arm 13118 preferably is coupled to the access device support portion 13102 and/or the support arm 13101, as discussed above. The extension arm 13118 of the viewing element support portion 13104 and the extension arm 13112 of the access device support portion 13102 each preferably define an opening 13122, 13124, respectively, at coupling locations on their proximal portions. The support arm can be coupled to the mount fixture 13100 at the coupling locations. In some embodiments, the viewing element support portion 13104 and the access device support portion 13102 can be held independently. For example, individual support arms 13101 can be provided for the viewing element support portion 13104 and the access device support portion 13102. A portion of the support arm assembly, such as, for example, a pin, can extend through the openings 13122, 13124 to couple the support arm and the mount fixture 13100 together.

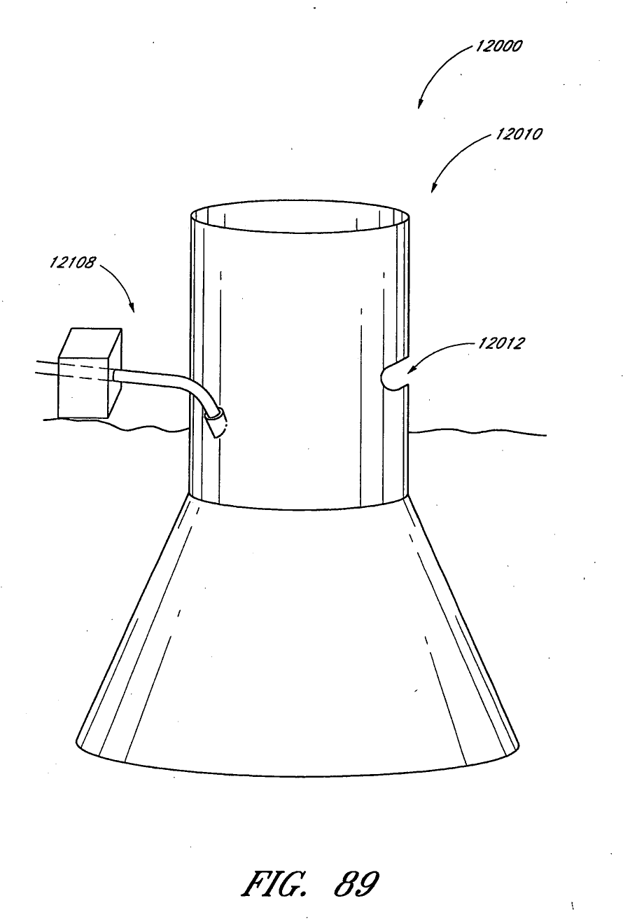

FIG. 91 shows another embodiment of an access assembly 14000 that can be incorporated into a surgical system, such as the system 10. The access assembly 14000 includes an access device 14010 coupled with a mount fixture 14100. The access assembly 13000 is similar to the access assembly 13000, shown in FIG. 90, except as described below. Similar features have similar reference numerals, except that they are in the 14000 range rather than the 13000 range as shown in FIG. 90. As shown in FIG. 91, the proximal portion of the access device 14010 preferably comprises a flange 14013 located below a midline 14015 of the access device 14010. In the illustrated embodiment, the access device support portion 14102 is not positioned directly adjacent the flange 14013. The access device support portion 14102 is positioned about a proximal portion of the access device 14010 above a skin surface 14017 of the patient. The flange 14013 is positioned below the skin surface 14017 of the patient in the illustrated embodiment.

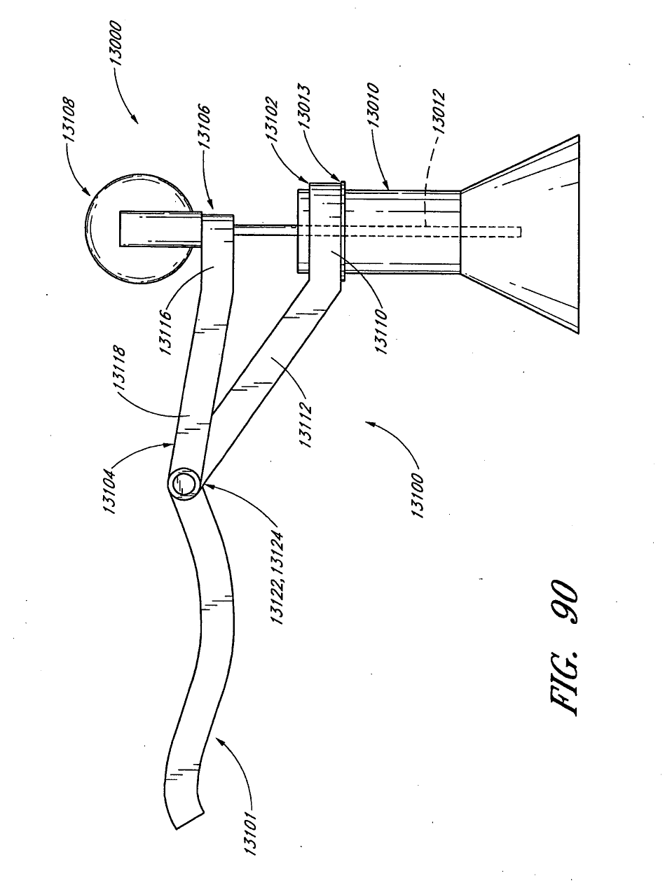

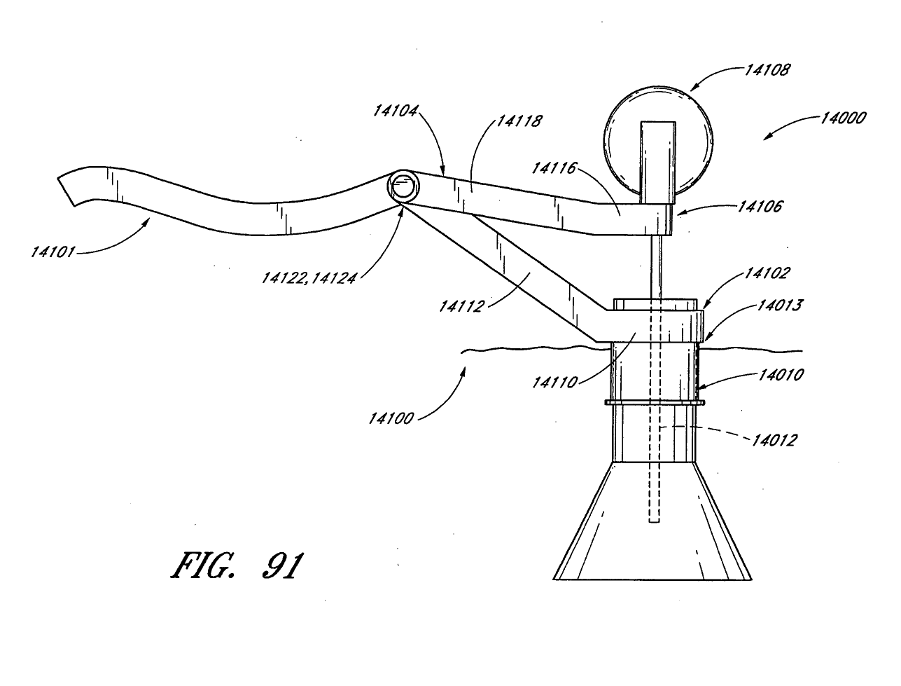

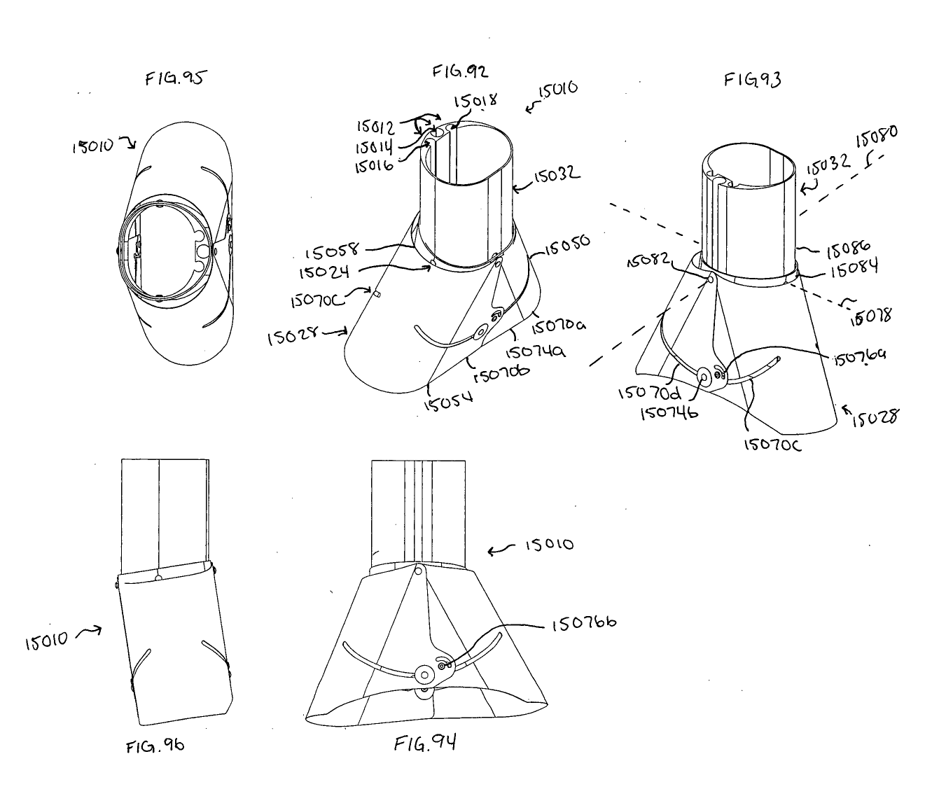

FIGS. 92-98 show additional embodiments of access devices and systems having one or more discrete locations for visualization or other instruments. FIGS. 92-96 show an access device or retractor 15010 that can be incorporated into a surgical system, such as the system 10. In the illustrated embodiment, the access device 15010 is similar to those access devices hereinbefore described, except as set forth below. The access device 15010 has instrument passages 15012, formed integrally with the body of the access device and extending longitudinally, that are configured so that a first instrument passage 15014 is flanked by a second instrument passage 15016 and a third instrument passage 15018 all along one side of the access device in one embodiment. The first instrument passage 15014 is configured with an opening facing the exterior of the access device 15010. The second instrument passage 15016 and the third instrument passage 15018 are configured with an opening facing a working channel defined by the proximal portion 15032. Instruments placed in the instrument passages may desirably be positioned such that they are at or beyond the surface of the inner or outer wall. In one embodiment, where a passage opens to an exterior of the access device, such opening need not be completely open. Additional openings can also be provided along an interior surface of the passage.

The instrument passages 15012 can be arranged in other configurations. For example, in other embodiments the second instrument passage 15016 may be flanked by the first instrument passage 15014 and the third instrument passage 15018. In these other embodiments, the first instrument passage 15014 may be configured with an opening facing the exterior of the access device 15010. Alternatively, the second instrument passage 15016 or the third instrument passage 15018 may be configured with an opening facing the exterior of the access device 15010. Other permutations regarding the ordering of the instrument passages 15012 and the configuration of the openings are contemplated. For example, some passages can be completely enclosed, as described above. In another example, some embodiments have one or two instrument passages 15012, while some other embodiments have more than three instrument passages 15012. In some embodiments, the instrument passages can have a plurality of openings along their length, for example, with closed off portions. In some embodiments, one or more openings form a slot. In some embodiments, one or more openings form a hole. In some embodiments, some openings can face the interior of the access device and other openings can face the exterior of the access device. For example, in some embodiments, a first opening faces an interior portion of the access device and a second opening faces an exterior portion of the access device. The openings as illustrated are partially circular or C-shaped, and are adapted to conform to closely hold instruments therein. Preferably openings are defined by a surface extending more than 180° around the opening. Passages can be placed at any desired location along the perimeter of the device, with desired openings to the interior or exterior of the device.

The openings allow drugs, chemicals, or other materials to be selectively introduced into the patient's body by using either the opening facing the exterior of the access device 15010 or the working channel defined by the access device 15010 or by using both types of openings. Fluids can also be selectively removed from the patient by placing a suction device in one of the instrument passages 15012 that has an opening facing the exterior or the interior of the access device 15010. The openings also allow the physician to observe the depth of an instrument inserted into the instrument passages 15012.

The instrument passages 15012 preferably are positioned within a thick portion of the wall forming the proximal portion 15032, but not in the distal portion 15028. However, these passages could extend the whole length of the access device in some embodiments. The wall preferably at least partially encloses the instrument passages. In the illustrated embodiment, the instrument passages 15012 are embedded within the wall forming the proximal portion 15032. Accordingly, the inner and outer transverse cross-sections are generally oblong, oval, or circular in shape and are not substantially interrupted by protrusions from the instrument passages 15012. This facilitates fabrication of devices for insertion into the working channel defined by the access device 15010 because of the relatively simplified geometry of the cross-sections.

The embodiment of the access device 15010 shown in FIGS. 92-96 is depicted in the expanded configuration. However, the access device 15010 is generally inserted into the patient in a contracted configuration. Once the access device 15010 is inserted, the distal portion 15028 can be expanded by rotating the first overlapping portion 15050 and the second overlapping portion 15054 outwards and in opposing directions. The expansion of the access device 15010 is facilitated by four arcuate slots 15070a, 15070b, 15070c and 15070d located on the overlapping portions 15050 and 15054. A first sliding rivet 15074a joins arcuate slots 15070a and 15070b together, and a second sliding rivet 15074b joins arcuate slots 15070c and 15070d together. The access device 15010 can be locked into the expanded configuration by using locking elements 15076a and 15076b. The locking elements 15076a and 15076b can be made by attaching a rotatable flange onto a first overlapping portion 15050 and fabricating a corresponding locking slot on the second overlapping portion. In the expanded configuration, the flange can be rotated into the locking slot, which reduces the likelihood that the access device 15010 will inadvertently collapse back into a collapsed configuration.

Another feature of the embodiment of the access device 15010 shown in FIGS. 92-96 is that the proximal portion 15032 is able to tilt along at least two different axes, a cephalad-caudal axis 15078 and a medial-lateral axis 15080. A pair of medial-lateral pivot points 15082 lies along the medial-lateral axis 15080 and allows the proximal portion 15032 to be tilted in either the cephalad or caudal direction. A pair of cephalad-caudal pivot points 15084 lies along the cephalad-caudal axis 15078 and allows the proximal portion 15032 to be tilted in either the medial or lateral direction. One pair of pivot points 15082 or 15084 attaches a gimbal ring 15086 to the proximal portion 15032 while the other pair of pivot points 15082 or 15084 attaches the gimbal ring 15086 to the distal portion 15028. The mobility of the proximal portion 15032 allows a physician to access a substantial portion of the working area exposed by the distal portion 15028.

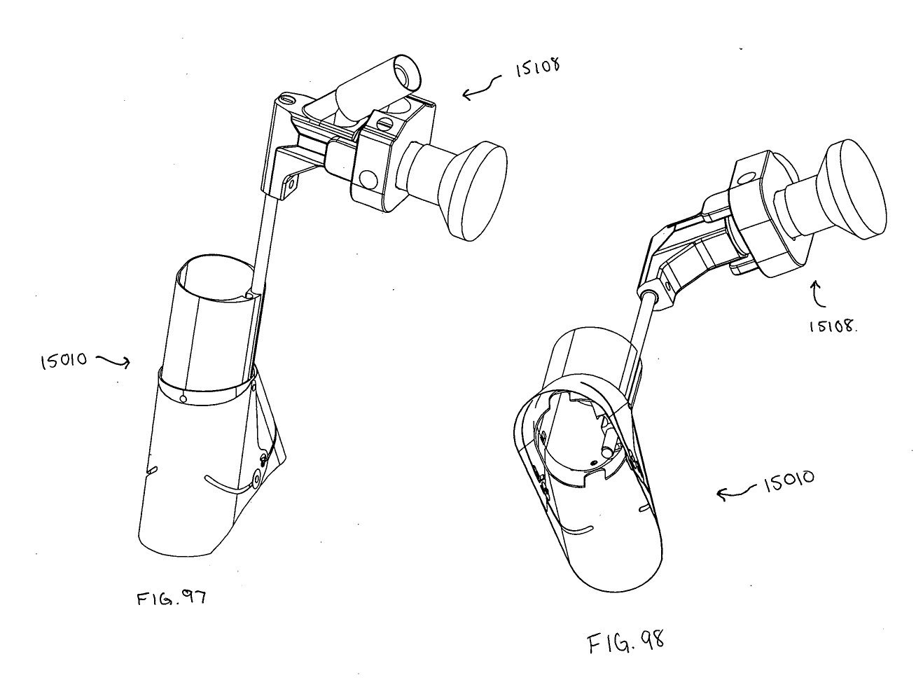

FIGS. 97-98 show an embodiment of the access device 15010 depicted in FIGS. 92-96 with a viewing element 15108 inserted into an instrument passage 15012. One embodiment of the access device 15010 has a first instrument passage 15014 that is configured to receive the viewing element 15108. The second instrument passage 15016 and third instrument passage 15018 are configured to receive surgical instruments and accessory devices such as, but not limited to, suction tubes, irrigation tubes, light tubes, electrocautery probes and retractors.

The access device 15010 in combination with a viewing element 15108 forms a system for accessing and visualizing a surgical location. Additionally, a mount fixture can be coupled to the access device 15010. The mount fixture preferably has fixture passages that correspond to the working channel and the instrument passages 15012 of the access device 15010.

A method for accessing and visualizing a surgical location with the access device 15010 is provided. The access device 15010 is provided in a contracted configuration for insertion into an incision at the surgical location. One preferable surgical location is a spinal location. Once the access device 15010 is inserted into the incision, the access device 15010 can be expanded into an expanded configuration. A viewing element 15108 configured to be inserted into an instrument passage 15012 is provided. The viewing element can be inserted into the instrument passage to visualize the surgical location. In some embodiments, accessory devices can be inserted into one or more of the other instrument passages where provided.

The various devices, methods and techniques described above provide a number of ways to carry out the invention. Of course, it is to be understood that not necessarily all objectives or advantages described may be achieved in accordance with any particular embodiment described herein. Also, although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, the invention is not intended to be limited by the specific disclosures of preferred embodiments herein.

What is claimed is:

1. A device for providing access to a surgical location within a patient, said device comprising:
an elongate body having a proximal portion and a distal portion, said elongate body defining a first passage for accessing the surgical location with surgical instruments, said elongate body having a contracted configuration for insertion into the patient and an expanded configuration for providing access to the surgical location, wherein the cross-sectional area of said first passage at a first location of the expanded configuration is greater than the cross-sectional area of said first passage at a second location of the expanded configuration; and
a second passage formed integrally with said elongate body, said second passage extending at least partially from the proximal portion toward the distal portion and sized and configured to receive a surgical instrument, said second passage being partially enclosed by a wall at least in said proximal portion.

2. The device of claim 1, wherein said first passage has a generally circular shaped cross section.

3. The device of claim 1, wherein said first passage has a generally oblong shaped cross section.

4. The device of claim 1, wherein said first passage has a generally oval shaped cross section.

5. The device of claim 1, wherein said second passage is configured with an opening along at least an exterior portion of the length of said second passage.

6. The device of claim 1, wherein said second passage is configured with an opening along at least an interior portion of the length of said second passage.

7. The device of claim 1, additionally comprising a third passage formed integrally with said elongate body, said third passage extending from the proximal portion toward the distal portion and sized and configured to receive a surgical instrument, said third passage being partially enclosed by a wall of said proximal portion.

8. The device of claim 7, wherein said second passage is configured with an opening along at least an exterior portion of the length of said second passage, and wherein said third passage is configured with an opening along at least an interior portion of the length of said third passage.

9. The device of claim 1, additionally comprising a fourth passage formed integrally with said elongate body, said fourth passage extending from the proximal portion toward the distal portion and sized and configured to receive a surgical instrument, said fourth passage being partially enclosed by a wall of said proximal portion.

10. The device of claim 1, wherein said proximal portion is configured to be articulated in generally a medial-lateral direction.

11. The device of claim 1, wherein said proximal portion is configured to be articulated in generally a cephalad-caudal direction.

12. The device of claim 1, wherein said proximal portion is configured to be articulated in generally both a medial-lateral direction and a cephalad-caudal direction.

13. A system for accessing and visualizing a surgical location, comprising:
a device having an elongate body defining a first passage for accessing the surgical location with surgical instruments and a second passage for visualizing the surgical location with a viewing element, said elongate body having a proximal portion and a distal portion, said elongate body having a contracted configuration for insertion into the patient and an expanded configuration for providing access to the surgical location, said second passage being partially enclosed by a wall at least in said proximal portion; and
a surgical instrument configured to be inserted into said second passage.

14. The system of claim 13 wherein said surgical instrument is a viewing element.

15. The system of claim 13, further comprising a mount fixture configured to be coupled to said device, said mount fixture defining a first fixture passage configured to be aligned with said first passage and a second fixture passage configured to be aligned with said second passage.

16. The system of claim 13, further comprising at least one additional passage configured to receive an accessory device, said additional passage being partially enclosed by a wall of said proximal portion.

17. The system of claim 16, wherein said accessory device is selected from a group consisting of a suction tube, an irrigation tube, a light tube, an electrocautery probe and a retractor.

18. A method of accessing a surgical location, comprising:
providing a device comprising an elongate body having a proximal portion and a distal portion, said elongate body defining a first passage and a second passage, said first passage extending through said elongate body through which surgical instruments can be inserted to a surgical location of a patient, said second passage being partially enclosed by a wall at least in said proximal portion, said second passage configured to receive a surgical instrument, said elongate body configurable to have an expanded configuration; and
configuring said elongate body for insertion into the patient.

19. The method of claim 18, further comprising:
inserting said device into said patient to said surgical location;
expanding said device to the expanded configuration, such that the cross-sectional area of said first passage at a first location is greater than the cross-sectional area of said first at a second location, wherein said first location is distal to said second location; and
positioning a surgical instrument in said second passage.

20. The method of claim 19, wherein the surgical instrument is a viewing element.

21. The method of claim 18, wherein said wall of said proximal portion partially encloses one or more additional passages, wherein said additional passages are configured to receive accessory devices.

22. The method of claim 21, further comprising inserting an accessory device into said one or more additional passages.

23. The method of claim 18, wherein the device is inserted at a spinal location.
